(19) United States
(12) Patent Application Publication
(10) Pub. No.: US 2012/0016002 A1
(43) Pub. Date: Jan. 19, 2012

(54) Patient Selection and Therapeutic Methods Using Markers of Prostaglandin Metabolism

(75) Inventors: Tracy Lawhon, Encinitas, CA (US); Sara L. Zaknoen, Carlsbad, CA (US); Thomas Estok, Carlsbad, CA (US); Mark Green, Charleston, SC (US)

(73) Assignee: TRAGARA PHARMACEUTICALS ,Inc., San Diego, CA (US)

(21) Appl. No.: 13/130,547

(22) PCT Filed: Jul. 11, 2008
(86) PCT No.: PCT/US2008/069892

§ 371 Date: Sep. 28, 2011

(51) Int. Cl. A61K 31/402; A61P 35/00; A61P 29/00; G01N 24/00

(52) U.S. Cl. 514/427; 436/106

(57) ABSTRACT

Disclosed herein are methods related generally to a patient selection process for identifying individuals for treatment of cancer, inflammation, pain, and/or related conditions.

### CROSS-REFERENCE

This application claims the benefit of U.S. Provisional Application No. 60/949,473, filed on Jul. 12, 2007, which is incorporated herein by reference in its entirety.

### FIELD OF THE INVENTION

Provided herein are methods related generally to a patient selection process for identifying individuals for treatment of cancer, inflammation, pain, and/or related conditions. Also provided herein are methods for selecting a patient for COX-2 inhibitor treatment and/or a combination of a COX-2 inhibitor and a second therapeutic agent treatment based upon a change in prostaglandin metabolite levels.

### BACKGROUND OF THE INVENTION

Prostaglandins (which include PGE₂, PGD₂, PGF_{2α}, PGI₂, and other related compounds) represent a diverse group of autocrine and paracrine hormones that are derived from the metabolism of fatty acids. They belong to a family of naturally occurring eicosanoids which are biosynthesized on demand from arachidonic acid. The production of prostaglandins begins with the liberation of arachidonic acid from membrane phospholipids by phospholipase A₂ in response to inflammatory stimuli. Arachidonic acid is converted to PGH₂ by the cyclooxygenase enzymes COX-1 and COX-2. Generally, it is believed that COX-1 is expressed constitutively in most tissues of the body and acts to maintain homeostatic processes. COX-2, in contrast, is mainly an inducible enzyme and is involved primarily in the regulation of inflammation. Under normal conditions, the eicosanoids are produced at low levels to serve as mediators of many cellular functions; however, the prostaglandins also play critical roles in pathophysiology. More particularly, inflammation is both initiated and maintained, at least in part, by the overproduction of prostaglandins in injured cells.

COX-2 is a cyclic endoperoxidase that catalyzes the rate-limiting step in prostaglandin (PG) synthesis and is frequently overexpressed in certain cancers. The relationship between enhanced cyclooxygenase expression and selected cancers is well-established. Over-expression of COX-2 has been noted in such cancers as cancers of the breast, head and neck, colon, brain, ovary, and prostate as well as non-small cell cancer of the lung. Moreover, COX-2 expression is nearly ubiquitous in human cancers and has been correlated with poor prognosis. Lee, Alice et al. “Inhibition of Cyclooxygenase-2 Disrupts Tumor Vascular Mural Cell Recruitment and Survival Signaling” Cancer Res. 66: 4378-84 (2006).

Tumors with up-regulation of COX-2 synthesize high levels of prostaglandin. High prostaglandin levels are associated with increased production of pro-angiogenic factors, altered immune responses, tumor cell proliferation, inhibited tumor cell apoptosis, and enhanced metastatic potential. Additionally, prostaglandin production increases tumor progression by altering cell morphology and increasing cell motility and migration. Gately, S. “The Contributions of Cyclooxygenase-2 to Tumor Angiogenesis” Cancer Metastasis Rev. 19:19-27 (2000). These findings suggest that increased COX-2 expression may play a significant role in the development and growth of cancers and possibly in the acquisition of an invasive and metastatic phenotype. Therefore, the inhibition of COX-2 may be useful in the treatment of cancer and/or cancer related disorders.

The overproduction of eicosanoids can be quantified relatively easily in cell culture by measuring parent PGs via immunoassay, HPLC, and mass spectrometry. However, measurement of endogenous prostaglandin production in patients is more challenging. It is generally accepted that an accurate index of endogenous eicosanoid production in humans is the measurement of excreted urinary metabolites. Catella, F. et al. “Measurement of renal and non-renal eicosanoid synthesis” Am. J. Med. 81 (1986): 23-29. Urinary 11α-hydroxy-9,15-dioxo-2,3,4,5-tetranor-prostane-1,20-dioic acid (PGE-M) is the major urinary metabolite of PGE₂. See Yang L., Huang Y H, Porta R, et al: Profound Reduction in Tumor Metastasis With Selective EP4 Receptor Antagonism. Cancer Res. 66, 9665-72, 2006. PGE-M is a surrogate marker for COX-2 and various studies have shown that increased PGE-M levels can be correlated to increased COX-2 expression and a poor prognosis for cancer patients.

### SUMMARY OF THE INVENTION

The present invention provides a method for enhancing the treatment of a subject having a condition wherein the level of expression of COX-2 plays a role. The invention provides a method comprising determining the level of a PGE2 metabolite (e.g., PGEM) in a sample obtained from a patient and establishing a therapeutic regimen including administration of a COX-2 selective inhibitor based on the level of the metabolite in the sample. The therapeutic regimen incorporates base metabolite levels and or changes in the metabolite level and is tailored such that therapeutic objectives are achieved while minimizing one or more of the side effects generally associated with treatments with COX-2 inhibitors.

The invention provides treatment methods that allow for therapeutic regimens involving one or more COX-2 inhibitors and at least one or more additional therapeutic agents or therapies which are tailored for higher likelihood for a successful therapy. For example, by determining the effect of the at least one or more additional therapeutic agents or therapies on COX-2 expression for a subject, the invention provides therapeutic methods wherein one or more COX-2 inhibitors are administered in combination with at least one or more additional agents or therapies resulting in enhanced outcomes for the subject. The invention also provides additional embodiments for treating the subject with multiple agents or therapies administered with a combination comprising a COX-2 inhibitor compound and at least one or more additional agents or therapies. The present invention contemplates using multiple agents or therapies at the same time, or in sequence, or co-administering with the combination to treat a condition or patient. Advantages of the invention also include reduction in the pain generally associated with cancer conditions and/or treatment side effects.

By incorporating measurements of metabolite levels, the invention provides methods for treatment with a combination comprising a COX-2 selective inhibitor and a second agent or therapy wherein administering the combination to the patient provides an enhanced treatment compared to administering the COX-2 selective inhibitor alone or the second agent or treatment alone. Some of the advantages of the invention include treatments which result in significant reduction in the levels of COX-2 expression. Reductions of 10%, 15%, 20%, 25%, 30%, 35% 40%, 45%, 50%, 55%, 60%, 65%, 70% and 75% of COX-2 expression upon treatment according to the invention are indicative of significantly improved prognosis.

The methods of the invention are also advantageous in that they provide methods for treatment with a combination comprising a COX-2 selective inhibitor and at least one or more additional agents or therapies wherein the combination may be administered at a lower dosage compared to administering the COX-2 selective inhibitor alone or the at least one or more additional agents or therapies alone. In one embodiment, the invention provides methods of treatment with a combination comprising a COX-2 selective inhibitor, at least one or more additional agents or therapies; and/or an optional third, or additional, or subsequent agent or therapy that is administered with the combination. The optional or additional third agent or therapy may include standard regimen of treatment.

In another embodiment, the invention provides a method for treating a subject with the combination comprising a COX-2 selective inhibitor compound and at least one or more additional agents or therapies; and/or an optional third, or additional, or subsequent agent or therapy wherein the combination and/or optional third, or additional, or subsequent agent or therapy are administered concurrently. In a further embodiment, the invention provides a method for treating a subject with the combination comprising a COX-2 selective inhibitor compound and at least one or more additional agents or therapies; and/or an optional third, or additional, or subsequent agent or therapy wherein the combination and/or optional third, or additional, or subsequent agent or therapy are administered sequentially. In yet a further embodiment, the invention provides a method for treating a subject wherein the additional or subsequent agent or therapy comprises at least between 1 and 4 additional agents or therapies.

In one embodiment, the invention provides a method of selecting a subject for therapy with the COX-2 selective inhibitor compound 2-(4-ethoxyphenyl)-4-methyl-1-(4-sulfamoylphenyl)pyrrole comprising: measuring the level of PGEM in a biological sample collected from the subject prior to administration of the compound 2-(4-ethoxyphenyl)-4-methyl-1-(4-sulfamoylphenyl)pyrrole, determining that the PGEM level in the sample is higher than a predetermined value and selecting the subject for therapy with the COX-2 selective inhibitor compound 2-(4-ethoxyphenyl)-4-methyl-1-(4-sulfamoylphenyl)pyrrole.

In another embodiment, the invention provides a method of selecting a subject for therapy with a combination comprising the COX-2 selective inhibitor compound 2-(4-ethoxyphenyl)-4-methyl-1-(4-sulfamoylphenyl)pyrrole and a second agent or therapy, the method comprising: measuring the level of PGEM in a biological sample collected from the subject prior to administration of the compound 2-(4-ethoxyphenyl)-4-methyl-1-(4-sulfamoylphenyl)pyrrole, determining that the PGEM level in the sample is higher than predetermined value and selecting the subject for therapy with the combination of the COX-2 selective inhibitor and second agent or therapy.

In yet another embodiment, the invention provides a method of selecting a subject for therapy with a combination comprising the COX-2 selective inhibitor compound 2-(4-ethoxyphenyl)-4-methyl-1-(4-sulfamoylphenyl)pyrrole and a second agent or therapy the method comprising: measuring the level of PGEM in a first biological sample collected from the subject prior to administration of the compound 2-(4-ethoxyphenyl)-4-methyl-1-(4-sulfamoylphenyl)pyrrole, measuring the level of PGEM in a second biological sample collected from the subject after administration of the second agent or therapy, and determining a ratio by dividing the level of PGEM in the second sample by the level of PGEM in the first sample, determining that the ratio is higher than a predetermined value and selecting the subject for therapy with the combination of the COX-2 selective inhibitor compound 2-(4-ethoxyphenyl)-4-methyl-1-(4-sulfamoylphenyl)pyrrole and the second agent or therapy.

In still another embodiment, the invention provides a method of selecting a subject for therapy with the COX-2 selective inhibitor compound 2-(4-ethoxyphenyl)-4-methyl-1-(4-sulfamoylphenyl)pyrrole comprising: measuring the level of PGE2 metabolite in a biological sample collected from the subject prior to administration of the compound 2-(4-ethoxyphenyl)-4-methyl-1-(4-sulfamoylphenyl)pyrrole, determining that the PGE2 metabolite level in the sample is higher than a predetermined value and selecting the subject for therapy with the COX-2 selective inhibitor compound 2-(4-ethoxyphenyl)-4-methyl-1-(4-sulfamoylphenyl)pyrrole.

In still another embodiment, the invention provides a method of selecting a subject for therapy with a combination comprising the COX-2 selective inhibitor compound 2-(4-ethoxyphenyl)-4-methyl-1-(4-sulfamoylphenyl)pyrrole and a second agent or therapy, the method comprising: measuring the level of PGE2 metabolite in a biological sample collected from the subject prior to administration of the compound 2-(4-ethoxyphenyl)-4-methyl-1-(4-sulfamoylphenyl)pyrrole, determining that the PGE2 metabolite level in the sample is higher than a predetermined value and selecting the subject for therapy with the combination of the COX-2 selective inhibitor compound 2-(4-ethoxyphenyl)-4-methyl-1-(4-sulfamoylphenyl)pyrrole and the second agent or therapy.

In yet another embodiment, the invention provides a method of selecting a subject for therapy with a combination comprising the COX-2 selective inhibitor compound 2-(4-ethoxyphenyl)-4-methyl-1-(4-sulfamoylphenyl)pyrrole and a second agent or therapy the method comprising: measuring the level of PGE2 metabolite in a first biological sample collected from the subject prior to administration of the compound 2-(4-ethoxyphenyl)-4-methyl-1-(4-sulfamoylphenyl)pyrrole, measuring the level of PGE2 metabolite in a second biological sample collected from the subject after administration of the second agent or therapy, and determining a ratio by dividing the level of PGE2 metabolite in the second sample by the level of PGE2 metabolite in the first sample, determining that the ratio is higher than a predetermined value and selecting the subject for therapy with the combination of the COX-2 selective inhibitor compound 2-(4-ethoxyphenyl)-4-methyl-1-(4-sulfamoylphenyl)pyrrole and the second agent or therapy.

In a further embodiment, the invention provides a method of treating a subject with the COX-2 selective inhibitor compound 2-(4-ethoxyphenyl)-4-methyl-1-(4-sulfamoylphenyl)pyrrole comprising: measuring the level of PGEM in a biological sample collected from the subject prior to administration of the compound 2-(4-ethoxyphenyl)-4-methyl-1-(4-sulfamoylphenyl)pyrrole, determining that the PGEM level in the sample is higher than a predetermined value and administering to the subject the COX-2 selective inhibitor 2-(4-ethoxyphenyl)-4-methyl-1-(4-sulfamoylphenyl)pyrrole.

In a still further embodiment, the invention provides a method of treating a subject with a combination comprising the COX-2 selective inhibitor compound 2-(4-ethoxyphenyl)-4-methyl-1-(4-sulfamoylphenyl)pyrrole and a second agent or therapy, the method comprising: measuring the level of PGEM in a biological sample collected from the subject prior to administration of the compound 2-(4-ethoxyphenyl)-4-methyl-1-(4-sulfamoylphenyl)pyrrole, determining that the PGEM level in the sample is higher than a predetermined value and administering to the subject the COX-2 selective inhibitor 2-(4-ethoxyphenyl)-4-methyl-1-(4-sulfamoylphenyl)pyrrole.

In a still further embodiment, the invention provides a method of treating a subject with a combination comprising the COX-2 selective inhibitor compound 2-(4-ethoxyphenyl)-4-methyl-1-(4-sulfamoylphenyl)pyrrole and a second agent or therapy the method comprising: measuring the level of PGEM in a first biological sample collected from the subject prior to administration of the compound 2-(4-ethoxyphenyl)-4-methyl-1-(4-sulfamoylphenyl)pyrrole, measuring the level of PGEM in a second biological sample collected from the subject after administration of the second agent or therapy, and determining a ratio by dividing the level of PGEM in the second sample by the level of PGEM in the first sample, determining that the ratio is higher than a predetermined value and administering to the subject the combination of the COX-2 selective inhibitor compound 2-(4-ethoxyphenyl)-4-methyl-1-(4-sulfamoylphenyl)pyrrole and the second agent or therapy.

In yet another embodiment, the invention provides a method of treating a subject with the COX-2 selective inhibitor compound 2-(4-ethoxyphenyl)-4-methyl-1-(4-sulfamoylphenyl)pyrrole comprising: measuring the level of PGE2 metabolite in a biological sample collected from the subject prior to administration of the compound 2-(4-ethoxyphenyl)-4-methyl-1-(4-sulfamoylphenyl)pyrrole, determining that the PGE2 metabolite level in the sample is higher than predetermined value and administering to the subject the COX-2 selective inhibitor 2-(4-ethoxyphenyl)-4-methyl-1-(4-sulfamoylphenyl)pyrrole.

In yet another embodiment, the invention provides a method of treating a subject with a combination comprising the COX-2 selective inhibitor compound 2-(4-ethoxyphenyl)-4-methyl-1-(4-sulfamoylphenyl)pyrrole and a second agent or therapy, the method comprising: measuring the level of PGE2 metabolite in a biological sample collected from the subject prior to administration of the compound 2-(4-ethoxyphenyl)-4-methyl-1-(4-sulfamoylphenyl)pyrrole, determining that the PGE2 metabolite level in the sample is higher than a predetermined value and administering to the subject the COX-2 selective inhibitor 2-(4-ethoxyphenyl)-4-methyl-1-(4-sulfamoylphenyl)pyrrole.

In still another embodiment, the invention provides a method of treating a subject with a combination comprising the COX-2 selective inhibitor compound 2-(4-ethoxyphenyl)-4-methyl-1-(4-sulfamoylphenyl)pyrrole and a second agent or therapy the method comprising: measuring the level of PGE2 metabolite in a first biological sample collected from the subject prior to administration of the compound 2-(4-ethoxyphenyl)-4-methyl-1-(4-sulfamoylphenyl)pyrrole, measuring the level of PGE2 metabolite in a second biological sample collected from the subject after administration of the second agent or therapy, and determining a ratio by dividing the level of PGE2 metabolite in the second sample by the level of PGE2 metabolite in the first sample, determining that the ratio is higher than a predetermined value and administering to the subject the combination of the COX-2 selective inhibitor compound 2-(4-ethoxyphenyl)-4-methyl-1-(4-sulfamoylphenyl)pyrrole and the second agent or therapy.

In a further embodiment, the invention provides a method of selecting a subject for therapy with a 1,2-diphenylpyrrole derivative COX-2 selective inhibitor comprising: measuring the level of PGEM in a biological sample collected from the subject prior to administration of the 1,2-diphenylpyrrole derivative COX-2 selective inhibitor, determining that the PGEM level in the sample is higher than a predetermined value and selecting the subject for therapy with the 1,2-diphenylpyrrole derivative COX-2 selective inhibitor.

In a still further embodiment, the invention provides a method of selecting a subject for therapy with a combination comprising a 1,2-diphenylpyrrole derivative COX-2 selective inhibitor and a second agent or therapy, the method comprising: measuring the level of PGEM in a biological sample collected from the subject prior to administration of the 1,2-diphenylpyrrole derivative COX-2 selective inhibitor, determining that the PGEM level in the sample is higher than predetermined value and selecting the subject for therapy with the 1,2-diphenylpyrrole derivative COX-2 selective inhibitor.

In yet another embodiment, the invention provides a method of selecting a subject for therapy with a combination comprising a 1,2-diphenylpyrrole derivative COX-2 selective inhibitor and a second agent or therapy the method comprising: measuring the level of PGEM in a first biological sample collected from the subject prior to administration of the 1,2-diphenylpyrrole derivative COX-2 selective inhibitor, measuring the level of PGEM in a second biological sample collected from the subject after administration of the second agent or therapy, and determining a ratio by dividing the level of PGEM in the second sample by the level of PGEM in the first sample, determining that the ratio is higher than a predetermined value and selecting the subject for therapy with the combination of the 1,2-diphenylpyrrole derivative COX-2 selective inhibitor and the second agent or therapy.

In still another embodiment, the invention provides a method of selecting a subject for therapy with a 1,2-diphenylpyrrole derivative COX-2 selective inhibitor comprising: measuring the level of PGE2 metabolite in a biological sample collected from the subject prior to administration of the 1,2-diphenylpyrrole derivative COX-2 selective inhibitor, determining that the PGE2 metabolite level in the sample is higher than a predetermined value and selecting the subject for therapy with the 1,2-diphenylpyrrole derivative COX-2 selective inhibitor.

In another embodiment, the invention provides a method of selecting a subject for therapy with a combination comprising a 1,2-diphenylpyrrole derivative COX-2 selective inhibitor and a second agent or therapy, the method comprising: measuring the level of PGE2 metabolite in a biological sample collected from the subject prior to administration of the 1,2-diphenylpyrrole derivative COX-2 selective inhibitor, determining that the PGE2 metabolite level in the sample is higher than a predetermined value and selecting the subject for therapy with the combination comprising the 1,2-diphenylpyrrole derivative COX-2 selective inhibitor and the second agent or therapy.

In another embodiment, the invention provides a method of selecting a subject for therapy with a combination comprising a 1,2-diphenylpyrrole derivative COX-2 selective inhibitor and a second agent or therapy the method comprising: measuring the level of PGE2 metabolite in a first biological sample collected from the subject prior to administration of the 1,2-diphenylpyrrole derivative COX-2 selective inhibitor, measuring the level of PGE2 metabolite in a second biological sample collected from the subject after administration of the second agent or therapy, and determining a ratio by dividing the level of PGE2 metabolite in the second sample by the level of PGE2 metabolite in the first sample, determining that the ratio is higher than a predetermined value and selecting the subject for therapy with the combination of the 1,2-diphenylpyrrole derivative COX-2 selective inhibitor and the second agent or therapy.

In still another embodiment, the invention provides a method of treating a subject with a 1,2-diphenylpyrrole derivative COX-2 selective inhibitor comprising: measuring the level of PGEM in a biological sample collected from the subject prior to administration of the 1,2-diphenylpyrrole derivative COX-2 selective inhibitor, determining that the PGEM level in the sample is higher than a predetermined value and administering to the subject the 1,2-diphenylpyrrole derivative COX-2 selective inhibitor.

In a further embodiment, the invention provides a method of treating a subject with a combination comprising a 1,2-diphenylpyrrole derivative COX-2 selective inhibitor and a second agent or therapy, the method comprising: measuring the level of PGEM in a biological sample collected from the subject prior to administration of the 1,2-diphenylpyrrole derivative COX-2 selective inhibitor, determining that the PGEM level in the sample is higher than a predetermined value and administering to the subject the combination comprising the 1,2-diphenylpyrrole derivative COX-2 selective inhibitor and the second agent or therapy.

In still another embodiment, the invention provides a method of treating a subject with a combination comprising a 1,2-diphenylpyrrole derivative COX-2 selective inhibitor and a second agent or therapy the method comprising: measuring the level of PGEM in a first biological sample collected from the subject prior to administration of the 1,2-diphenylpyrrole derivative COX-2 selective inhibitor, measuring the level of PGEM in a second biological sample collected from the subject after administration of the second agent or therapy, and determining a ratio by dividing the level of PGEM in the second sample by the level of PGEM in the first sample, determining that the ratio is higher than a predetermined value and administering to the subject the combination of the 1,2-diphenylpyrrole derivative COX-2 selective inhibitor and the second agent or therapy.

In yet another embodiment, the invention provides a method of treating a subject with a 1,2-diphenylpyrrole derivative COX-2 selective inhibitor comprising: measuring the level of PGE2 metabolite in a biological sample collected from the subject prior to administration of the 1,2-diphenylpyrrole derivative COX-2 selective inhibitor, determining that the PGE2 metabolite level in the sample is higher than predetermined value and administering to the 1,2-diphenylpyrrole derivative COX-2 selective inhibitor.

In yet another embodiment, the invention provides a method of treating a subject with a combination comprising a 1,2-diphenylpyrrole derivative COX-2 selective inhibitor and a second agent or therapy, the method comprising: measuring the level of PGE2 metabolite in a biological sample collected from the subject prior to administration of the 1,2-diphenylpyrrole derivative COX-2 selective inhibitor, determining that the PGE2 metabolite level in the sample is higher than a predetermined value and administering to the subject the combination comprising the 1,2-diphenylpyrrole derivative COX-2 selective inhibitor and the second agent or therapy.

In yet another embodiment, the invention provides a method of treating a subject with a combination comprising a 1,2-diphenylpyrrole derivative COX-2 selective inhibitor and a second agent or therapy the method comprising: measuring the level of PGE2 metabolite in a first biological sample collected from the subject prior to administration of the 1,2-diphenylpyrrole derivative COX-2 selective inhibitor, measuring the level of PGE2 metabolite in a second biological sample collected from the subject after administration of the second agent or therapy, and determining a ratio by dividing the level of PGE2 metabolite in the second sample by the level of PGE2 metabolite in the first sample, determining that the ratio is higher than a predetermined value and administering to the subject the combination of the 1,2-diphenylpyrrole derivative COX-2 selective inhibitor and the second agent or therapy.

In a further embodiment, the invention provides a method of selecting a subject for therapy with a COX-2 selective inhibitor compound the method comprising: measuring the level of PGEM in a biological sample collected from the subject prior to administration of the compound, determining that the PGEM level in the sample is higher than about 7 ng/mg for a female subject or higher than about 12 ng/mg for a male subject, and selecting the subject for therapy with the COX-2 selective inhibitor.

In a still further embodiment, the invention provides a method of selecting a subject for therapy with a 1,2-diphenylpyrrole derivative COX-2 selective inhibitor compound the method comprising: measuring the level of PGEM in a biological sample collected from the subject prior to administration of the compound, determining that the PGEM level in the sample is higher than about 7 ng/mg for a female subject or higher than about 12 ng/mg for a male subject, and selecting the subject for therapy with the COX-2 selective inhibitor.

In yet another embodiment, the invention provides a method of selecting a subject for therapy with the COX-2 selective inhibitor compound 2-(4-ethoxyphenyl)-4-methyl-1-(4-sulfamoylphenyl)pyrrole comprising: measuring the level of PGEM in a biological sample collected from the subject prior to administration of the compound, determining that the PGEM level in the sample is higher than about 7 ng/mg for a female subject or higher than about 12 ng/mg for a male subject, and selecting the subject for therapy with the COX-2 selective inhibitor.

In still another embodiment, the invention provides a method of selecting a subject for therapy with a combination of a COX-2 selective inhibitor compound and a second agent or therapy the method comprising: measuring the level of PGEM in a biological sample collected from the subject prior to administration of the compound, determining that the PGEM level in the sample is higher than about 7 ng/mg for a female subject or higher than about 12 ng/mg for a male subject, and selecting the subject for therapy with the combination COX-2 selective inhibitor.

In yet another embodiment, the invention provides a method of selecting a subject for therapy with a combination of a 1,2-diphenylpyrrole derivative COX-2 selective inhibitor compound and a second agent or therapy the method comprising: measuring the level of PGEM in a biological sample collected from the subject prior to administration of the compound, determining that the PGEM level in the sample is higher than about 7 ng/mg for a female subject or higher than about 12 ng/mg for a male subject, and selecting the subject for therapy with the combination COX-2 selective inhibitor.

In another embodiment, the invention provides a method of selecting a subject for therapy with a combination comprising the COX-2 selective inhibitor compound 2-(4-ethoxyphenyl)-4-methyl-1-(4-sulfamoylphenyl)pyrrole and a second agent or therapy, the method comprising: measuring the level of PGEM in a biological sample collected from the subject prior to administration of the compound, determining that the PGEM level in the sample is higher than about 7 ng/mg for a female subject or higher than about 12 ng/mg for a male subject, and selecting the subject for therapy with the COX-2 selective inhibitor.

In a further embodiment, the invention provides a method of selecting a subject for therapy with a combination comprising a COX-2 selective inhibitor compound and a second agent or therapy the method comprising: measuring the level of PGEM in a first biological sample collected from the subject prior to administration of the compound, measuring the level of PGEM in a second biological sample collected from the subject after administration of the second agent or therapy, and determining a ratio by dividing the level of PGEM in the second sample by the level of PGEM in the first sample, determining that the ratio is higher than about 1.2 and selecting the subject for therapy with the combination of the COX-2 selective inhibitor and the second agent or therapy.

In yet another embodiment, the invention provides a method of selecting a subject for therapy with a combination comprising a 1,2-diphenylpyrrole derivative COX-2 selective inhibitor compound and a second agent or therapy the method comprising: measuring the level of PGEM in a first biological sample collected from the subject prior to administration of the compound, measuring the level of PGEM in a second biological sample collected from the subject after administration of the second agent or therapy, and determining a ratio by dividing the level of PGEM in the second sample by the level of PGEM in the first sample, determining that the ratio is higher than about 1.2 and selecting the subject for therapy with the combination of the COX-2 selective inhibitor and the second agent or therapy.

In a further embodiment, the invention provides a method of selecting a subject for therapy with a combination comprising the COX-2 selective inhibitor compound 2-(4-ethoxyphenyl)-4-methyl-1-(4-sulfamoylphenyl)pyrrole and a second agent or therapy the method comprising: measuring the level of PGEM in a first biological sample collected from the subject prior to administration of the compound, measuring the level of PGEM in a second biological sample collected from the subject after administration of the second agent or therapy, and determining a ratio by dividing the level of PGEM in the second sample by the level of PGEM in the first sample, determining that the ratio is higher than about 1.2 and selecting the subject for therapy with the combination of the COX-2 selective inhibitor compound 2-(4-ethoxyphenyl)-4-methyl-1-(4-sulfamoylphenyl)pyrrole and the second agent or therapy.

In still another embodiment, the invention provides a method of selecting a subject for therapy with a COX-2 selective inhibitor compound the method comprising: measuring the level of PGE2 metabolite in a biological sample collected from the subject prior to administration of the compound, determining that the PGE2 metabolite level in the sample is higher than about 7 ng/mg for a female subject or higher than about 12 ng/mg for a male subject, and selecting the subject for therapy with the COX-2 selective inhibitor.

In yet another embodiment, the invention provides a method of selecting a subject for therapy with a 1,2-diphenylpyrrole derivative COX-2 selective inhibitor compound the method comprising: measuring the level of PGE2 metabolite in a biological sample collected from the subject prior to administration of the compound, determining that the PGE2 metabolite level in the sample is higher than about 7 ng/mg for a female subject or higher than about 12 ng/mg for a male subject, and selecting the subject for therapy with the COX-2 selective inhibitor.

In still another embodiment, the invention provides a method of selecting a subject for therapy with the COX-2 selective inhibitor compound 2-(4-ethoxyphenyl)-4-methyl-1-(4-sulfamoylphenyl)pyrrole comprising: measuring the level of PGE2 metabolite in a biological sample collected from the subject prior to administration of the compound, determining that the PGE2 metabolite level in the sample is higher than about 7 ng/mg for a female subject or higher than about 12 ng/mg for a male subject, and selecting the subject for therapy with the COX-2 selective inhibitor.

In yet another embodiment, the invention provides a method of selecting a subject for therapy with a combination of a COX-2 selective inhibitor compound and a second agent or therapy the method comprising: measuring the level of PGE2 metabolite in a biological sample collected from the subject prior to administration of the compound, determining that the PGE2 metabolite level in the sample is higher than about 7 ng/mg for a female subject or higher than about 12 ng/mg for a male subject, and selecting the subject for therapy with the combination COX-2 selective inhibitor.

In yet a further embodiment, the invention provides a method of selecting a subject for therapy with a combination of a 1,2-diphenylpyrrole derivative COX-2 selective inhibitor compound and a second agent or therapy the method comprising: measuring the level of PGE2 metabolite in a biological sample collected from the subject prior to administration of the compound, determining that the PGE2 metabolite level in the sample is higher than about 7 ng/mg for a female subject or higher than about 12 ng/mg for a male subject, and selecting the subject for therapy with the combination COX-2 selective inhibitor.

In still another embodiment, the invention provides a method of selecting a subject for therapy with a combination comprising the COX-2 selective inhibitor compound 2-(4-ethoxyphenyl)-4-methyl-1-(4-sulfamoylphenyl)pyrrole and a second agent or therapy, the method comprising: measuring the level of PGE2 metabolite in a biological sample collected from the subject prior to administration of the compound, determining that the PGE2 metabolite level in the sample is higher than about 7 ng/mg for a female subject or higher than about 12 ng/mg for a male subject, and selecting the subject for therapy with the COX-2 selective inhibitor.

In yet another embodiment, the invention provides a method of selecting a subject for therapy with a combination comprising a COX-2 selective inhibitor compound and a second agent or therapy the method comprising: measuring the level of PGE2 metabolite in a first biological sample collected from the subject prior to administration of the compound, measuring the level of PGE2 metabolite in a second biological sample collected from the subject after administration of the second agent or therapy, and determining a ratio by dividing the level of PGE2 metabolite in the second sample by the level of PGE2 metabolite in the first sample, determining that the ratio is higher than about 1.2 and selecting the subject for therapy with the combination of the COX-2 selective inhibitor and the second agent or therapy.

In still another embodiment, the invention provides a method of selecting a subject for therapy with a combination comprising a 1,2-diphenylpyrrole derivative COX-2 selective inhibitor compound and a second agent or therapy the method comprising: measuring the level of PGE2 metabolite in a first biological sample collected from the subject prior to administration of the compound, measuring the level of PGE2 metabolite in a second biological sample collected from the subject after administration of the second agent or therapy, and determining a ratio by dividing the level of PGE2 metabolite in the second sample by the level of PGE2 metabolite in the first sample, determining that the ratio is higher than about 1.2 and selecting the subject for therapy with the combination of the COX-2 selective inhibitor and the second agent or therapy.

In yet another embodiment, the invention provides a method of selecting a subject for therapy with a combination comprising the COX-2 selective inhibitor compound 2-(4-ethoxyphenyl)-4-methyl-1-(4-sulfamoylphenyl)pyrrole and a second agent or therapy the method comprising: measuring the level of PGE2 metabolite in a first biological sample collected from the subject prior to administration of the compound, measuring the level of PGE2 metabolite in a second biological sample collected from the subject after administration of the second agent or therapy, and determining a ratio by dividing the level of PGE2 metabolite in the second sample by the level of PGE2 metabolite in the first sample, determining that the ratio is higher than about 1.2 and selecting the subject for therapy with the combination of the COX-2 selective inhibitor compound 2-(4-ethoxyphenyl)-4-methyl-1-(4-sulfamoylphenyl)pyrrole and the second agent or therapy.

In a further embodiment, the invention provides a method of treating a subject with a COX-2 selective inhibitor compound the method comprising: measuring the level of PGEM in a biological sample collected from the subject prior to administration of the compound, determining that the PGEM level in the sample is higher than about 7 ng/mg for a female subject or higher than about 12 ng/mg for a male subject, and administering to the subject the COX-2 selective inhibitor.

In a still further embodiment, the invention provides a method of treating a subject with a 1,2-diphenylpyrrole derivative COX-2 selective inhibitor compound the method comprising: measuring the level of PGEM in a biological sample collected from the subject prior to administration of the compound, determining that the PGEM level in the sample is higher than about 7 ng/mg for a female subject or higher than about 12 ng/mg for a male subject, and administering to the subject the COX-2 selective inhibitor.

In yet another embodiment, the invention provides a method of treating a subject with the COX-2 selective inhibitor compound 2-(4-ethoxyphenyl)-4-methyl-1-(4-sulfamoylphenyl)pyrrole comprising: measuring the level of PGEM in a biological sample collected from the subject prior to administration of the compound, determining that the PGEM level in the sample is higher than about 7 ng/mg for a female subject or higher than about 12 ng/mg for a male subject, and administering to the subject the COX-2 selective inhibitor.

In a further embodiment, the invention provides a method of treating a subject with a combination of a COX-2 selective inhibitor compound and a second agent or therapy the method comprising: measuring the level of PGEM in a biological sample collected from the subject prior to administration of the compound, determining that the PGEM level in the sample is higher than about 7 ng/mg for a female subject or higher than about 12 ng/mg for a male subject, and administering to the subject the combination comprising the COX-2 selective inhibitor and the second agent or therapy.

In another embodiment, the invention provides a method of treating a subject with a combination of a 1,2-diphenylpyrrole derivative COX-2 selective inhibitor compound and a second agent or therapy the method comprising: measuring the level of PGEM in a biological sample collected from the subject prior to administration of the compound, determining that the PGEM level in the sample is higher than about 7 ng/mg for a female subject or higher than about 12 ng/mg for a male subject, and administering to the subject the combination comprising the COX-2 selective inhibitor and the second agent or therapy.

In still another embodiment, the invention provides a method of treating a subject with a combination comprising the COX-2 selective inhibitor compound 2-(4-ethoxyphenyl)-4-methyl-1-(4-sulfamoylphenyl)pyrrole and a second agent or therapy, the method comprising: measuring the level of PGEM in a biological sample collected from the subject prior to administration of the compound, determining that the PGEM level in the sample is higher than about 7 ng/mg for a female subject or higher than about 12 ng/mg for a male subject, and administering to the subject the COX-2 selective inhibitor.

In yet another embodiment, the invention provides a method of treating a subject with a combination comprising a COX-2 selective inhibitor compound and a second agent or therapy the method comprising: measuring the level of PGEM in a first biological sample collected from the subject prior to administration of the compound, measuring the level of PGEM in a second biological sample collected from the subject after administration of the second agent or therapy, and determining a ratio by dividing the level of PGEM in the second sample by the level of PGEM in the first sample, determining that the ratio is higher than about 1.2 and administering to the subject the combination of the COX-2 selective inhibitor and the second agent or therapy.

In one embodiment, the invention provides a method of treating a subject with a combination comprising a 1,2-diphenylpyrrole derivative COX-2 selective inhibitor compound and a second agent or therapy the method comprising: measuring the level of PGEM in a first biological sample collected from the subject prior to administration of the compound, measuring the level of PGEM in a second biological sample collected from the subject after administration of the second agent or therapy, and determining a ratio by dividing the level of PGEM in the second sample by the level of PGEM in the first sample, determining that the ratio is higher than about 1.2 and administering to the subject the combination of the COX-2 selective inhibitor and the second agent or therapy.

In another embodiment, the invention provides a method of treating a subject with a combination comprising the COX-2 selective inhibitor compound 2-(4-ethoxyphenyl)-4-methyl-1-(4-sulfamoylphenyl)pyrrole and a second agent or therapy the method comprising: measuring the level of PGEM in a first biological sample collected from the subject prior to administration of the compound, measuring the level of PGEM in a second biological sample collected from the subject after administration of the second agent or therapy, and determining a ratio by dividing the level of PGEM in the second sample by the level of PGEM in the first sample, determining that the ratio is higher than about 1.2 and administering to the subject the combination of the COX-2 selective inhibitor compound 2-(4-ethoxyphenyl)-4-methyl-1-(4-sulfamoylphenyl)pyrrole and the second agent or therapy.

In yet another embodiment, the invention provides a method of treating a subject with a COX-2 selective inhibitor compound the method comprising: measuring the level of PGE2 metabolite in a biological sample collected from the subject prior to administration of the compound, determining that the PGE2 metabolite level in the sample is higher than about 7 ng/mg for a female subject or higher than about 12 ng/mg for a male subject, and administering to the subject the COX-2 selective inhibitor.

In still another embodiment, the invention provides a method of treating a subject with a 1,2-diphenylpyrrole derivative COX-2 selective inhibitor compound the method comprising: measuring the level of PGE2 metabolite in a biological sample collected from the subject prior to administration of the compound, determining that the PGE2 metabolite level in the sample is higher than about 7 ng/mg for a female subject or higher than about 12 ng/mg for a male subject, and administering to the subject the COX-2 selective inhibitor.

In a further embodiment, the invention provides a method of treating a subject with the COX-2 selective inhibitor compound 2-(4-ethoxyphenyl)-4-methyl-1-(4-sulfamoylphenyl)pyrrole comprising: the method comprising: measuring the level of PGE2 metabolite in a biological sample collected from the subject prior to administration of the compound, determining that the PGE2 metabolite level in the sample is higher than about 7 ng/mg for a female subject or higher than about 12 ng/mg for a male subject, and administering to the subject the COX-2 selective inhibitor.

In a still further embodiment, the invention provides a method of treating a subject with a combination of a COX-2 selective inhibitor compound and a second agent or therapy the method comprising: measuring the level of PGE2 metabolite in a biological sample collected from the subject prior to administration of the compound, determining that the PGE2 metabolite level in the sample is higher than about 7 ng/mg for a female subject or higher than about 12 ng/mg for a male subject, and administering to the subject the combination COX-2 selective inhibitor.

In another embodiment, the invention provides a method of treating a subject with a combination of a 1,2-diphenylpyrrole derivative COX-2 selective inhibitor compound and a second agent or therapy the method comprising: measuring the level of PGE2 metabolite in a biological sample collected from the subject prior to administration of the compound, determining that the PGE2 metabolite level in the sample is higher than about 7 ng/mg for a female subject or higher than about 12 ng/mg for a male subject, and administering to the subject the combination COX-2 selective inhibitor.

In yet another embodiment, the invention provides a method of treating a subject with a combination comprising the COX-2 selective inhibitor compound 2-(4-ethoxyphenyl)-4-methyl-1-(4-sulfamoylphenyl)pyrrole and a second agent or therapy, the method comprising: measuring the level of PGE2 metabolite in a biological sample collected from the subject prior to administration of the compound, determining that the PGE2 metabolite level in the sample is higher than about 7 ng/mg for a female subject or higher than about 12 ng/mg for a male subject, and administering to the subject the COX-2 selective inhibitor.

In still another embodiment, the invention provides a method of treating a subject with a combination comprising a COX-2 selective inhibitor compound and a second agent or therapy the method comprising: measuring the level of PGE2 metabolite in a first biological sample collected from the subject prior to administration of the compound, measuring the level of PGE2 metabolite in a second biological sample collected from the subject after administration of the second agent or therapy, and determining a ratio by dividing the level of PGE2 metabolite in the second sample by the level of PGE2 metabolite in the first sample, determining that the ratio is higher than about 1.2 and administering to the subject the combination of the COX-2 selective inhibitor and the second agent or therapy.

In a further embodiment, the invention provides a method of treating a subject with a combination comprising a 1,2-diphenylpyrrole derivative COX-2 selective inhibitor compound and a second agent or therapy the method comprising: measuring the level of PGE2 metabolite in a first biological sample collected from the subject prior to administration of the compound, measuring the level of PGE2 metabolite in a second biological sample collected from the subject after administration of the second agent or therapy, and determining a ratio by dividing the level of PGE2 metabolite in the second sample by the level of PGE2 metabolite in the first sample, determining that the ratio is higher than about 1.2 and administering to the subject the combination of the COX-2 selective inhibitor and the second agent or therapy.

In still another embodiment, the invention provides a method of treating a subject with a combination comprising the COX-2 selective inhibitor compound 2-(4-ethoxyphenyl)-4-methyl-1-(4-sulfamoylphenyl)pyrrole and a second agent or therapy the method comprising: measuring the level of PGE2 metabolite in a first biological sample collected from the subject prior to administration of the compound, measuring the level of PGE2 metabolite in a second biological sample collected from the subject after administration of the second agent or therapy, and determining a ratio by dividing the level of PGE2 metabolite in the second sample by the level of PGE2 metabolite in the first sample, determining that the ratio is higher than about 1.2 and administering to the subject the combination of the COX-2 selective inhibitor compound 2-(4-ethoxyphenyl)-4-methyl-1-(4-sulfamoylphenyl)pyrrole and the second agent or therapy.

In yet another embodiment, the invention provides a method of assessing reduction of COX-2 expression in a subject undergoing therapy with the COX-2 selective inhibitor compound 2-(4-ethoxyphenyl)-4-methyl-1-(4-sulfamoylphenyl)pyrrole the method comprising: measuring the PGEM level in the subjects urine at least a first and a second point in time to produce at least a first level of PGEM and a second level of PGEM, wherein the at least first and second points in time are separated from each other by an interval of at least about five days, and wherein a decrease in the second level of PGEM compared to the first level of PGEM is indicative of decreased COX-2 expression.

In a further embodiment, the invention provides a method of assessing reduction of COX-2 expression in a subject undergoing therapy with the COX-2 selective inhibitor compound 2-(4-ethoxyphenyl)-4-methyl-1-(4-sulfamoylphenyl)pyrrole the method comprising: measuring the PGE2 metabolite level in the subjects urine at least a first and a second point in time to produce at least a first level of PGE2 metabolite and a second level of PGE2 metabolite, wherein the at least first and second points in time are separated from each other by an interval of at least about five days, and wherein a decrease in the second level of PGE2 metabolite compared to the first level of PGE2 metabolite is indicative of decreased COX-2 expression.

In a still further embodiment, the invention provides a method of assessing reduction of COX-2 expression in a subject undergoing therapy with a combination comprising the COX-2 selective inhibitor compound 2-(4-ethoxyphenyl)-4-methyl-1-(4-sulfamoylphenyl)pyrrole and a second agent or therapy the method comprising: measuring the PGEM level in the subjects urine at least a first and a second point in time to produce at least a first level of PGEM and a second level of PGEM, wherein the at least first and second points in time are separated from each other by an interval of at least about five days, and wherein a decrease in the second level of PGEM compared to the first level of PGEM is indicative of decreased COX-2 expression.

In yet another embodiment, the invention provides a method of assessing reduction of COX-2 expression in a subject undergoing therapy with a combination comprising the COX-2 selective inhibitor compound 2-(4-ethoxyphenyl)-4-methyl-1-(4-sulfamoylphenyl)pyrrole and a second agent or therapy the method comprising: measuring the PGE2 metabolite level in the subjects urine at least a first and a second point in time to produce at least a first level of PGE2 metabolite and a second level of PGE2 metabolite, wherein the at least first and second points in time are separated from each other by an interval of at least about five days, and wherein a decrease in the second level of PGE2 metabolite compared to the first level of PGE2 metabolite is indicative of decreased COX-2 expression.

In a further embodiment, the invention provides a method of assessing reduction of COX-2 expression in a subject undergoing therapy with a 1,2-diphenylpyrrole derivative COX-2 selective inhibitor the method comprising: measuring the PGEM level in the subjects urine at least a first and a second point in time to produce at least a first level of PGEM and a second level of PGEM, wherein the at least first and second points in time are separated from each other by an interval of at least about five days, and wherein a decrease in the second level of PGEM compared to the first level of PGEM is indicative of decreased COX-2 expression.

In yet another embodiment, the invention provides a method of assessing reduction of COX-2 expression in a subject undergoing therapy with a 1,2-diphenylpyrrole derivative COX-2 selective inhibitor the method comprising: measuring the PGE2 metabolite level in the subjects urine at least a first and a second point in time to produce at least a first level of PGE2 metabolite and a second level of PGE2 metabolite, wherein the at least first and second points in time are separated from each other by an interval of at least about five days, and wherein a decrease in the second level of PGE2 metabolite compared to the first level of PGE2 metabolite is indicative of decreased COX-2 expression.

In still another embodiment, the invention provides a method of assessing reduction of COX-2 expression in a subject undergoing therapy with a combination comprising a 1,2-diphenylpyrrole derivative COX-2 selective inhibitor and a second agent or therapy the method comprising: measuring the PGEM level in the subjects urine at least a first and a second point in time to produce at least a first level of PGEM and a second level of PGEM, wherein the at least first and second points in time are separated from each other by an interval of at least about five days, and wherein a decrease in the second level of PGEM compared to the first level of PGEM is indicative of decreased COX-2 expression.

In a further embodiment, the invention provides a method of assessing reduction of COX-2 expression in a subject undergoing therapy with a combination comprising a 1,2-diphenylpyrrole derivative COX-2 selective inhibitor and a second agent or therapy the method comprising: measuring the PGE2 metabolite level in the subjects urine at least a first and a second point in time to produce at least a first level of PGE2 metabolite and a second level of PGE2 metabolite, wherein the at least first and second points in time are separated from each other by an interval of at least about five days, and wherein a decrease in the second level of PGE2 metabolite compared to the first level of PGE2 metabolite is indicative of decreased COX-2 expression.

In a further embodiment, the invention provides a method of assessing response to treatment in a subject undergoing therapy with the COX-2 selective inhibitor compound 2-(4-ethoxyphenyl)-4-methyl-1-(4-sulfamoylphenyl)pyrrole the method comprising: measuring the PGEM level in the subjects urine at least a first and a second point in time to produce at least a first level of PGEM and a second level of PGEM, wherein the at least first and second points in time are separated from each other by an interval of at least about five days, and wherein a decrease in the second level of PGEM compared to the first level of PGEM is indicative of positive response to treatment.

In a further embodiment, the invention provides a method of assessing response to treatment in a subject undergoing therapy with the COX-2 selective inhibitor compound 2-(4-ethoxyphenyl)-4-methyl-1-(4-sulfamoylphenyl)pyrrole the method comprising: measuring the PGE2 metabolite level in the subjects urine at least a first and a second point in time to produce at least a first level of PGE2 metabolite and a second level of PGE2 metabolite, wherein the at least first and second points in time are separated from each other by an interval of at least about five days, and wherein a decrease in the second level of PGE2 metabolite compared to the first level of PGE2 metabolite is indicative of positive response to treatment.

In still another embodiment, the invention provides a method of assessing response to treatment in a subject undergoing therapy with a combination comprising the COX-2 selective inhibitor compound 2-(4-ethoxyphenyl)-4-methyl-1-(4-sulfamoylphenyl)pyrrole and a second agent or therapy the method comprising: measuring the PGEM level in the subjects urine at least a first and a second point in time to produce at least a first level of PGEM and a second level of PGEM, wherein the at least first and second points in time are separated from each other by an interval of at least about five days, and wherein a decrease in the second level of PGEM compared to the first level of PGEM is indicative of positive response to treatment.

In a further embodiment, the invention provides a method of assessing response to treatment in a subject undergoing therapy with a combination comprising the COX-2 selective inhibitor compound 2-(4-ethoxyphenyl)-4-methyl-1-(4-sulfamoylphenyl)pyrrole and a second agent or therapy the method comprising: measuring the PGE2 metabolite level in the subjects urine at least a first and a second point in time to produce at least a first level of PGE2 metabolite and a second level of PGE2 metabolite, wherein the at least first and second points in time are separated from each other by an interval of at least about five days, and wherein a decrease in the second level of PGE2 metabolite compared to the first level of PGE2 metabolite is indicative of positive response to treatment.

In yet another embodiment, the invention provides a method of assessing response to treatment in a subject undergoing therapy with a 1,2-diphenylpyrrole derivative COX-2 selective inhibitor the method comprising: measuring the PGEM level in the subjects urine at least a first and a second point in time to produce at least a first level of PGEM and a second level of PGEM, wherein the at least first and second points in time are separated from each other by an interval of at least about five days, and wherein a decrease in the second level of PGEM compared to the first level of PGEM is indicative of positive response to treatment.

In still another embodiment, the invention provides a method of assessing response to treatment in a subject undergoing therapy with a 1,2-diphenylpyrrole derivative COX-2 selective inhibitor the method comprising: measuring the PGE2 metabolite level in the subjects urine at least a first and a second point in time to produce at least a first level of PGE2 metabolite and a second level of PGE2 metabolite, wherein the at least first and second points in time are separated from each other by an interval of at least about five days, and wherein a decrease in the second level of PGE2 metabolite compared to the first level of PGE2 metabolite is indicative of positive response to treatment.

In still another embodiment, the invention provides a method of assessing response to treatment in a subject undergoing therapy with a combination comprising a 1,2-diphenylpyrrole derivative COX-2 selective inhibitor and a second agent or therapy the method comprising: measuring the PGEM level in the subjects urine at least a first and a second point in time to produce at least a first level of PGEM and a second level of PGEM, wherein the at least first and second points in time are separated from each other by an interval of at least about five days, and wherein a decrease in the second level of PGEM compared to the first level of PGEM is indicative positive response to treatment.

In still another embodiment, the invention provides a method of assessing response to treatment in a subject undergoing therapy with a combination comprising a 1,2-diphenylpyrrole derivative COX-2 selective inhibitor and a second agent or therapy the method comprising: measuring the PGE2 metabolite level in the subjects urine at least a first and a second point in time to produce at least a first level of PGE2 metabolite and a second level of PGE2 metabolite, wherein the at least first and second points in time are separated from each other by an interval of at least about five days, and wherein a decrease in the second level of PGE2 metabolite compared to the first level of PGE2 metabolite is indicative of positive response to treatment.

In yet another embodiment, the invention provides a method for treating a patient in need with a combination of 2-(4-ethoxyphenyl)-4-methyl-1-(4-sulfamoylphenyl)pyrrole and a second therapeutic agent, wherein a PGE-M level of a patient sample is higher than a pre-determined PGE-M level, the method comprising, administering a therapeutically effective amount of the combination of 244-ethoxyphenyl)-4-methyl-1-(4-sulfamoylphenyl)pyrrole and the second therapeutic agent.

In another embodiment, the invention provides a method for treating a patient whose condition results in an elevated prostaglandin level, wherein a PGE-M level of a patient sample is higher than a pre-determined PGE-M level, the method comprising, administering a therapeutically effective amount of 2-(4-ethoxyphenyl)-4-methyl-1-(4-sulfamoylphenyl)pyrrole.

In still another embodiment, the invention provides a method for treating a patient whose condition results in an elevated prostaglandin expression level, wherein a PGE-M level of a sample from a patient is higher than a pre-determined PGE-M level, the method comprising, administering a therapeutically effective amount of a combination of 2-(4-ethoxyphenyl)-4-methyl-1-(4-sulfamoylphenyl)pyrrole and a second therapeutic agent.

In still another embodiment, the invention provides a method of selecting a patient for treatment with a combination of 2-(4-ethoxyphenyl)-4-methyl-1-(4-sulfamoylphenyl)pyrrole and a second therapeutic agent, the method comprising: determining a first PGE-M level of a first sample from the patient; administering the second therapeutic agent to the patient; determining a second PGE-M level of a second sample from the patient; and selecting the patient for treatment with the combination of 2-(4-ethoxyphenyl)-4-methyl-1-(4-sulfamoylphenyl)pyrrole and the second therapeutic agent, provided that the second PGE-M level is greater than the first PGE-M level.

In yet another embodiment, the invention provides a method of selecting a patient for treatment with a combination of 2-(4-ethoxyphenyl)-4-methyl-1-(4-sulfamoylphenyl)pyrrole and a second therapeutic agent, comprising: determining a PGE-M level of a patient sample; and selecting the patient for treatment with the combination of 2-(4-ethoxyphenyl)-4-methyl-1-(4 sulfamoylphenyl)pyrrole and the second therapeutic agent provided that the PGE-M level is above a pre-determined PGE-M level.

In a further embodiment, the invention provides a method for treating a patient in need with 2-(4-ethoxyphenyl)-4-methyl-1-(4-sulfamoylphenyl)pyrrole, wherein a PGE-M level of a patient sample is higher than a pre-determined PGE-M level, the method comprising administering a therapeutically effective amount of 2-(4-ethoxyphenyl)-4-methyl-1-(4-sulfamoylphenyl)pyrrole.

In yet another embodiment, the invention provides a method for treating a patient whose condition results in an elevated prostaglandin level, wherein a PGE-M level of a patient sample is higher than a pre-determined PGE-M level, the method comprising, administering a therapeutically effective amount of 2-(4-ethoxyphenyl)-4-methyl-1-(4-sulfamoylphenyl)pyrrole.

In another embodiment, the invention provides a method of treating a subject with a combination comprising the COX-2 selective inhibitor compound 2-(4-ethoxyphenyl)-4-methyl-1-(4-sulfamoylphenyl)pyrrole and a second agent or therapy, the method comprising: measuring the level of PGEM in a biological sample collected from the subject prior to administration of the compound, determining that the PGEM level in the sample is higher than a predetermined value and administering to the subject the COX-2 selective inhibitor; wherein administering the combination to the patient provides an enhanced treatment compared to administering the COX-2 selective inhibitor alone or the second agent or treatment alone.

In another embodiment, the invention provides a method of treating a subject with a combination comprising the COX-2 selective inhibitor compound 2-(4-ethoxyphenyl)-4-methyl-1-(4-sulfamoylphenyl)pyrrole and a second agent or therapy, the method comprising: measuring the level of PGEM in a biological sample collected from the subject prior to administration of the compound, determining that the PGEM level in the sample is higher than a predetermined value and administering to the subject the COX-2 selective inhibitor wherein the combination is administered at a lower dosage compared to administering the COX-2 selective inhibitor alone or the second agent or treatment alone.

In another embodiment, the invention provides a method of treating a subject with a combination comprising the COX-2 selective inhibitor compound 2-(4-ethoxyphenyl)-4-methyl-1-(4-sulfamoylphenyl)pyrrole and a second agent or therapy, the method comprising: measuring the level of PGEM in a biological sample collected from the subject prior to administration of the compound, determining that the PGEM level in the sample is higher than a predetermined value and administering to the subject the COX-2 selective inhibitor wherein administering the combination to the patient reduces side effects compared to administering the COX-2 selective inhibitor alone or the second agent or treatment alone.

In another embodiment, the invention provides a method of treating a subject with a combination comprising the COX-2 selective inhibitor compound 2-(4-ethoxyphenyl)-4-methyl-1-(4-sulfamoylphenyl)pyrrole and a second agent or therapy, the method comprising: measuring the level of PGEM in a biological sample collected from the subject prior to administration of the compound, determining that the PGEM level in the sample is higher than a predetermined value and administering to the subject the COX-2 selective inhibitor wherein administering the combination to the patient reduces resistance to the second agent compared to administering the second agent or treatment alone.

In another embodiment, the invention provides a method of selecting a subject for therapy with a combination comprising the COX-2 selective inhibitor compound 2-(4-ethoxyphenyl)-4-methyl-1-(4-sulfamoylphenyl)pyrrole and a second agent or therapy the method comprising: measuring the level of PGEM in a first biological sample collected from the subject prior to administration of the compound, measuring the level of PGEM in a second biological sample collected from the subject after administration of the second agent or therapy, and determining a ratio by dividing the level of PGEM in the second sample by the level of PGEM in the first sample, determining that the ratio is higher than a predetermined value and selecting the subject for therapy with the combination of the COX-2 selective inhibitor compound 2-(4-ethoxyphenyl)-4-methyl-1-(4-sulfamoylphenyl)pyrrole and the second agent or therapy; wherein administering the combination to the patient provides an enhanced treatment compared to administering the COX-2 selective inhibitor alone or the second agent or treatment alone.

In another embodiment, the invention provides a method of selecting a subject for therapy with a combination comprising the COX-2 selective inhibitor compound 2-(4-ethoxyphenyl)-4-methyl-1-(4-sulfamoylphenyl)pyrrole and a second agent or therapy the method comprising: measuring the level of PGEM in a first biological sample collected from the subject prior to administration of the compound, measuring the level of PGEM in a second biological sample collected from the subject after administration of the second agent or therapy, and determining a ratio by dividing the level of PGEM in the second sample by the level of PGEM in the first sample, determining that the ratio is higher than a predetermined value and selecting the subject for therapy with the combination of the COX-2 selective inhibitor compound 2-(4-ethoxyphenyl)-4-methyl-1-(4-sulfamoylphenyl)pyrrole and the second agent or therapy wherein the combination is administered at a lower dosage compared to administering the COX-2 selective inhibitor alone or the second agent or treatment alone.

In another embodiment, the invention provides a method of selecting a subject for therapy with a combination comprising the COX-2 selective inhibitor compound 2-(4-ethoxyphenyl)-4-methyl-1-(4-sulfamoylphenyl)pyrrole and a second agent or therapy the method comprising: measuring the level of PGEM in a first biological sample collected from the subject prior to administration of the compound, measuring the level of PGEM in a second biological sample collected from the subject after administration of the second agent or therapy, and determining a ratio by dividing the level of PGEM in the second sample by the level of PGEM in the first sample, determining that the ratio is higher than a predetermined value and selecting the subject for therapy with the combination of the COX-2 selective inhibitor compound 2-(4-ethoxyphenyl)-4-methyl-1-(4-sulfamoylphenyl)pyrrole and the second agent or therapy wherein administering the combination to the patient reduces side effects compared to administering the COX-2 selective inhibitor alone or the second agent or treatment alone.

In another embodiment, the invention provides a method of selecting a subject for therapy with a combination comprising the COX-2 selective inhibitor compound 2-(4-ethoxyphenyl)-4-methyl-1-(4-sulfamoylphenyl)pyrrole and a second agent or therapy the method comprising: measuring the level of PGEM in a first biological sample collected from the subject prior to administration of the compound, measuring the level of PGEM in a second biological sample collected from the subject after administration of the second agent or therapy, and determining a ratio by dividing the level of PGEM in the second sample by the level of PGEM in the first sample, determining that the ratio is higher than a predetermined value and selecting the subject for therapy with the combination of the COX-2 selective inhibitor compound 2-(4-ethoxyphenyl)-4-methyl-1-(4-sulfamoylphenyl)pyrrole and the second agent or therapy wherein administering the combination to the patient reduces resistance to the second agent compared to administering the second agent or treatment alone.

In another embodiment, the invention provides a method of treating a subject with a combination comprising a 1,2-diphenylpyrrole derivative COX-2 selective inhibitor and a second agent or therapy, the method comprising: measuring the level of PGEM in a biological sample collected from the subject prior to administration of the compound, determining that the PGEM level in the sample is higher than a predetermined value and administering to the subject the 1,2-diphenylpyrrole derivative COX-2 selective inhibitor; wherein administering the combination to the patient provides an enhanced treatment compared to administering the COX-2 selective inhibitor alone or the second agent or treatment alone.

In another embodiment, the invention provides a method of treating a subject with a combination comprising a 1,2-diphenylpyrrole derivative COX-2 selective inhibitor and a second agent or therapy, the method comprising: measuring the level of PGEM in a biological sample collected from the subject prior to administration of the compound, determining that the PGEM level in the sample is higher than a predetermined value and administering to the subject the 1,2-diphenylpyrrole derivative COX-2 selective inhibitor wherein the combination is administered at a lower dosage compared to administering the COX-2 selective inhibitor alone or the second agent or treatment alone.

In another embodiment, the invention provides a method of treating a subject with a combination comprising a 1,2-diphenylpyrrole derivative COX-2 selective inhibitor and a second agent or therapy, the method comprising: measuring the level of PGEM in a biological sample collected from the subject prior to administration of the compound, determining that the PGEM level in the sample is higher than a predetermined value and administering to the subject the 1,2-diphenylpyrrole derivative COX-2 selective inhibitor wherein administering the combination to the patient reduces side effects compared to administering the COX-2 selective inhibitor alone or the second agent or treatment alone.

In another embodiment, the invention provides a method of treating a subject with a combination comprising a 1,2-diphenylpyrrole derivative COX-2 selective inhibitor and a second agent or therapy, the method comprising: measuring the level of PGEM in a biological sample collected from the subject prior to administration of the compound, determining that the PGEM level in the sample is higher than a predetermined value and administering to the subject the 1,2-diphenylpyrrole derivative COX-2 selective inhibitor wherein administering the combination to the patient reduces resistance to the second agent compared to administering the second agent or treatment alone.

In another embodiment, the invention provides a method of selecting a subject for therapy with a combination comprising a 1,2-diphenylpyrrole derivative COX-2 selective inhibitor and a second agent or therapy the method comprising: measuring the level of PGEM in a first biological sample collected from the subject prior to administration of the compound, measuring the level of PGEM in a second biological sample collected from the subject after administration of the second agent or therapy, and determining a ratio by dividing the level of PGEM in the second sample by the level of PGEM in the first sample, determining that the ratio is higher than a predetermined value and selecting the subject for therapy with the combination of the 1,2-diphenylpyrrole derivative COX-2 selective inhibitor and the second agent or therapy; wherein administering the combination to the patient provides an enhanced treatment compared to administering the COX-2 selective inhibitor alone or the second agent or treatment alone.

In another embodiment, the invention provides a method of selecting a subject for therapy with a combination comprising a 1,2-diphenylpyrrole derivative COX-2 selective inhibitor and a second agent or therapy the method comprising: measuring the level of PGEM in a first biological sample collected from the subject prior to administration of the compound, measuring the level of PGEM in a second biological sample collected from the subject after administration of the second agent or therapy, and determining a ratio by dividing the level of PGEM in the second sample by the level of PGEM in the first sample, determining that the ratio is higher than a predetermined value and selecting the subject for therapy with the combination of the 1,2-diphenylpyrrole derivative COX-2 selective inhibitor and the second agent or therapy wherein the combination is administered at a lower dosage compared to administering the COX-2 selective inhibitor alone or the second agent or treatment alone.

In another embodiment, the invention provides a method of selecting a subject for therapy with a combination comprising a 1,2-diphenylpyrrole derivative COX-2 selective inhibitor and a second agent or therapy the method comprising: measuring the level of PGEM in a first biological sample collected from the subject prior to administration of the compound, measuring the level of PGEM in a second biological sample collected from the subject after administration of the second agent or therapy, and determining a ratio by dividing the level of PGEM in the second sample by the level of PGEM in the first sample, determining that the ratio is higher than a predetermined value and selecting the subject for therapy with the combination of the 1,2-diphenylpyrrole derivative COX-2 selective inhibitor and the second agent or therapy wherein administering the combination to the patient reduces side effects compared to administering the COX-2 selective inhibitor alone or the second agent or treatment alone.

In another embodiment, the invention provides a method of treating a subject with a combination comprising a 1,2-diphenylpyrrole derivative COX-2 selective inhibitor and a second agent or therapy the method comprising: measuring the level of PGEM in a first biological sample collected from the subject prior to administration of the compound, measuring the level of PGEM in a second biological sample collected from the subject after administration of the second agent or therapy, and determining a ratio by dividing the level of PGEM in the second sample by the level of PGEM in the first sample, determining that the ratio is higher than a predetermined value and administering to the subject the combination of the 1,2-diphenylpyrrole derivative COX-2 selective inhibitor and the second agent or therapy wherein administering the combination to the patient reduces side effects compared to administering the COX-2 selective inhibitor alone or the second agent or treatment alone.

In one embodiment, the invention provides a method of selecting a subject for therapy with the COX-2 selective inhibitor compound 2-(4-ethoxyphenyl)-4-methyl-1-(4-sulfamoylphenyl)pyrrole the method comprising: measuring the level of PGEM in a first biological sample collected from the subject prior to administration of the COX-2 selective inhibitor 2-(4-ethoxyphenyl)-4-methyl-1-(4-sulfamoylphenyl)pyrrole, measuring the level of PGEM in a second biological sample collected from the subject at least five days after administration of the selective COX-2 inhibitor, and calculating a ratio by dividing the level of PGEM in the second sample by the level of PGEM in the first sample, determining that the ratio is lower than a predetermined value and selecting the subject for therapy with the COX-2 selective inhibitor compound 2-(4-ethoxyphenyl)-4-methyl-1-(4-sulfamoylphenyl)pyrrole.

Advantageous embodiments of the invention include a method of selecting a subject for therapy with the COX-2 selective inhibitor compound 2-(4-ethoxyphenyl)-4-methyl-1-(4-sulfamoylphenyl)pyrrole the method comprising: measuring the level of PGEM in a first biological sample collected from the subject prior to administration of the COX-2 selective inhibitor 2-(4-ethoxyphenyl)-4-methyl-1-(4-sulfamoylphenyl)pyrrole, measuring the level of PGEM in a second biological sample collected from the subject at least five days after administration of the selective COX-2 inhibitor, and calculating a reduction in the PGEM level in the second sample compared to the level of PGEM in the first sample, determining that the reduction is 25% or greater and selecting the subject for therapy with the COX-2 selective inhibitor compound 2-(4-ethoxyphenyl)-4-methyl-1-(4-sulfamoylphenyl)pyrrole.

Advantageous embodiments of the invention include a method of selecting a subject for therapy with the COX-2 selective inhibitor compound 2-(4-ethoxyphenyl)-4-methyl-1-(4-sulfamoylphenyl)pyrrole the method comprising: measuring the level of PGEM in a first biological sample collected from the subject prior to administration of the COX-2 selective inhibitor, measuring the level of PGEM in a second biological sample collected from the subject at least five days after administration of the selective COX-2 inhibitor 2-(4-ethoxyphenyl)-4-methyl-1-(4-sulfamoylphenyl)pyrrole, and calculating a reduction in the PGEM level in the second sample compared to the level of PGEM in the first sample, determining that the reduction is 50% or greater and selecting the subject for therapy with the COX-2 selective inhibitor compound 2-(4-ethoxyphenyl)-4-methyl-1-(4-sulfamoylphenyl)pyrrole.

Advantageous embodiments of the invention include a method of selecting a subject for therapy with the COX-2 selective inhibitor compound 2-(4-ethoxyphenyl)-4-methyl-1-(4-sulfamoylphenyl)pyrrole the method comprising: measuring the level of PGEM in a first biological sample collected from the subject prior to administration of the COX-2 selective inhibitor 2-(4-ethoxyphenyl)-4-methyl-1-(4-sulfamoylphenyl)pyrrole, measuring the level of PGEM in a second biological sample collected from the subject at least five days after administration of the selective COX-2 inhibitor 2-(4-ethoxyphenyl)-4-methyl-1-(4-sulfamoylphenyl)pyrrole, and calculating a reduction in the PGEM level in the second sample compared to the level of PGEM in the first sample, determining that the reduction is 75% or greater and selecting the subject for therapy with the COX-2 selective inhibitor compound 2-(4-ethoxyphenyl)-4-methyl-1-(4-sulfamoylphenyl)pyrrole.

In another embodiment, the invention provides a method of selecting a subject for therapy with the COX-2 selective inhibitor compound 2-(4-ethoxyphenyl)-4-methyl-1-(4-sulfamoylphenyl)pyrrole the method comprising: measuring the level of PGE2 metabolite in a first biological sample collected from the subject prior to administration of the compound 2-(4-ethoxyphenyl)-4-methyl-1-(4-sulfamoylphenyl)pyrrole, measuring the level of PGE2 metabolite in a second biological sample collected from the subject at least five days after administration of the compound 2-(4-ethoxyphenyl)-4-methyl-1-(4-sulfamoylphenyl)pyrrole, and calculating a ratio by dividing the level of PGE2 metabolite in the second sample by the level of PGE2 metabolite in the first sample, determining that the ratio is lower than a predetermined value and selecting the subject for therapy with the COX-2 selective inhibitor compound 2-(4-ethoxyphenyl)-4-methyl-1-(4-sulfamoylphenyl)pyrrole.

In another embodiment, the invention provides a method of treating a subject with the COX-2 selective inhibitor compound 2-(4-ethoxyphenyl)-4-methyl-1-(4-sulfamoylphenyl)pyrrole the method comprising: measuring the level of PGEM in a first biological sample collected from the subject prior to administration of the compound 2-(4-ethoxyphenyl)-4-methyl-1-(4-sulfamoylphenyl)pyrrole, measuring the level of PGEM in a second biological sample collected from the subject at least five days after administration of the compound 2-(4-ethoxyphenyl)-4-methyl-1-(4-sulfamoylphenyl)pyrrole, and calculating a ratio by dividing the level of PGEM in the second sample by the level of PGEM in the first sample, determining that the ratio is lower than a predetermined value and administering to the subject the COX-2 selective inhibitor compound 2-(4-ethoxyphenyl)-4-methyl-1-(4-sulfamoylphenyl)pyrrole.

In another embodiment, the invention provides a method of treating a subject with the COX-2 selective inhibitor compound 2-(4-ethoxyphenyl)-4-methyl-1-(4-sulfamoylphenyl)pyrrole the method comprising: measuring the level of PGE2 metabolite in a first biological sample collected from the subject prior to administration of the compound, measuring the level of PGE2 metabolite in a second biological sample collected from the subject at least five days after administration of the compound 2-(4-ethoxyphenyl)-4-methyl-1-(4-sulfamoylphenyl)pyrrole, and calculating a ratio by dividing the level of PGE2 metabolite in the second sample by the level of PGE2 metabolite in the first sample, determining that the ratio is lower than a predetermined value and administering to the subject the COX-2 selective inhibitor compound 2-(4-ethoxyphenyl)-4-methyl-1-(4-sulfamoylphenyl)pyrrole.

In another embodiment, the invention provides a method of selecting a subject for therapy with a 1,2-diphenylpyrrole derivative COX-2 selective inhibitor the method comprising: measuring the level of PGEM in a first biological sample collected from the subject prior to administration of the 1,2-diphenylpyrrole derivative COX-2 selective inhibitor, measuring the level of PGEM in a second biological sample collected from the subject at least five days after administration of the 1,2-diphenylpyrrole derivative COX-2 selective inhibitor, and calculating a ratio by dividing the level of PGEM in the second sample by the level of PGEM in the first sample, determining that the ratio is lower than a predetermined value and selecting the subject for therapy with the 1,2-diphenylpyrrole derivative COX-2 selective inhibitor.

In another embodiment, the invention provides a method of selecting a subject for therapy with a 1,2-diphenylpyrrole derivative COX-2 selective inhibitor the method comprising: measuring the level of PGE2 metabolite in a first biological sample collected from the subject prior to administration of 1,2-diphenylpyrrole derivative COX-2 selective inhibitor, measuring the level of PGE2 metabolite in a second biological sample collected from the subject at least five days after administration of the 1,2-diphenylpyrrole derivative COX-2 selective inhibitor, and calculating a ratio by dividing the level of PGE2 metabolite in the second sample by the level of PGE2 metabolite in the first sample, determining that the ratio is lower than a predetermined value and selecting the subject for therapy with the 1,2-diphenylpyrrole derivative COX-2 selective inhibitor.

In another embodiment, the invention provides a method of treating a subject with a 1,2-diphenylpyrrole derivative COX-2 selective inhibitor the method comprising: measuring the level of PGEM in a first biological sample collected from the subject prior to administration of the 1,2-diphenylpyrrole derivative COX-2 selective inhibitor, measuring the level of PGEM in a second biological sample collected from the subject at least five days after administration of the 1,2-diphenylpyrrole derivative COX-2 selective inhibitor, and calculating a ratio by dividing the level of PGEM in the second sample by the level of PGEM in the first sample, determining that the ratio is lower than a predetermined value and administering to the subject the 1,2-diphenylpyrrole derivative COX-2 selective inhibitor.

In another embodiment, the invention provides a method of treating a subject with a 1,2-diphenylpyrrole derivative COX-2 selective inhibitor the method comprising: measuring the level of PGE2 metabolite in a first biological sample collected from the subject prior to administration of the 1,2-diphenylpyrrole derivative COX-2 selective inhibitor, measuring the level of PGE2 metabolite in a second biological sample collected from the subject at least five days after administration of 1,2-diphenylpyrrole derivative COX-2 selective inhibitor, and calculating a ratio by dividing the level of PGE2 metabolite in the second sample by the level of PGE2 metabolite in the first sample, determining that the ratio is lower than a predetermined value and administering to the subject the 1,2-diphenylpyrrole derivative COX-2 selective inhibitor.

In another embodiment, the invention provides a method of selecting a subject for therapy with the COX-2 selective inhibitor compound 2-(4-ethoxyphenyl)-4-methyl-1-(4-sulfamoylphenyl)pyrrole the method comprising: measuring the level of PGEM in a first biological sample collected from the subject prior to administration of the compound 2-(4-ethoxyphenyl)-4-methyl-1-(4-sulfamoylphenyl)pyrrole, measuring the level of PGEM in a second biological sample collected from the subject at least five days after administration of the compound 2-(4-ethoxyphenyl)-4-methyl-1-(4-sulfamoylphenyl)pyrrole, and calculating a ratio by dividing the level of PGEM in the second sample by the level of PGEM in the first sample, determining that the ratio is lower than about 0.8 and selecting the subject for therapy with the COX-2 selective inhibitor compound 244-ethoxyphenyl)-4-methyl-1-(4-sulfamoylphenyl)pyrrole.

In another embodiment, the invention provides a method of selecting a subject for therapy with a 1,2-diphenylpyrrole derivative COX-2 selective inhibitor compound the method comprising: measuring the level of PGEM in a first biological sample collected from the subject prior to administration of the 1,2-diphenylpyrrole derivative COX-2 selective inhibitor compound, measuring the level of PGEM in a second biological sample collected from the subject at least five days after administration of the 1,2-diphenylpyrrole derivative COX-2 selective inhibitor compound, and calculating a ratio by dividing the level of PGEM in the second sample by the level of PGEM in the first sample, determining that the ratio is lower than about 0.8 and selecting the subject for therapy with the 1,2-diphenylpyrrole derivative COX-2 selective inhibitor.

In another embodiment, the invention provides a method of selecting a subject for therapy with the COX-2 selective inhibitor compound 2-(4-ethoxyphenyl)-4-methyl-1-(4-sulfamoylphenyl)pyrrole the method comprising: measuring the level of PGE2 metabolite in a first biological sample collected from the subject prior to administration of the compound 2-(4-ethoxyphenyl)-4-methyl-1-(4-sulfamoylphenyl)pyrrole, measuring the level of PGE2 metabolite in a second biological sample collected from the subject at least five days after administration of the compound 2-(4-ethoxyphenyl)-4-methyl-1-(4-sulfamoylphenyl)pyrrole, and calculating a ratio by dividing the level of PGE2 metabolite in the second sample by the level of PGE2 metabolite in the first sample, determining that the ratio is lower than about 0.8 and selecting the subject for therapy with the COX-2 selective inhibitor compound 2-(4-ethoxyphenyl)-4-methyl-1-(4-sulfamoylphenyl)pyrrole.

In another embodiment, the invention provides a method of selecting a subject for therapy with a 1,2-diphenylpyrrole derivative COX-2 selective inhibitor compound the method comprising: measuring the level of PGE2 metabolite in a first biological sample collected from the subject prior to administration of the 1,2-diphenylpyrrole derivative COX-2 selective inhibitor compound, measuring the level of PGE2 metabolite in a second biological sample collected from the subject at least five days after administration of the 1,2-diphenylpyrrole derivative COX-2 selective inhibitor compound, and calculating a ratio by dividing the level of PGEM in the second sample by the level of PGE2 metabolite in the first sample, determining that the ratio is lower than about 0.8 and selecting the subject for therapy with the 1,2-diphenylpyrrole derivative COX-2 selective inhibitor.

In another embodiment, the invention provides a method of treating a subject with the COX-2 selective inhibitor compound 2-(4-ethoxyphenyl)-4-methyl-1-(4-sulfamoylphenyl)pyrrole the method comprising: measuring the level of PGEM in a first biological sample collected from the subject prior to administration of the compound, measuring the level of PGEM in a second biological sample collected from the subject at least five days after administration of the compound 2-(4-ethoxyphenyl)-4-methyl-1-(4-sulfamoylphenyl)pyrrole, and calculating a ratio by dividing the level of PGEM in the second sample by the level of PGEM in the first sample, determining that the ratio is lower than about 0.8 and administering to the subject the COX-2 selective inhibitor compound 2-(4-ethoxyphenyl)-4-methyl-1-(4-sulfamoylphenyl)pyrrole.

In another embodiment, the invention provides a method of treating a subject with a 1,2-diphenylpyrrole derivative COX-2 selective inhibitor compound the method comprising: measuring the level of PGE2 metabolite in a first biological sample collected from the subject prior to administration of the 1,2-diphenylpyrrole derivative COX-2 selective inhibitor compound, measuring the level of PGE2 metabolite in a second biological sample collected from the subject at least five days after administration of the 1,2-diphenylpyrrole derivative COX-2 selective inhibitor compound, and calculating a ratio by dividing the level of PGE2 metabolite in the second sample by the level of PGE2 metabolite in the first sample, determining that the ratio is lower than about 0.8 and administering to the subject the 1,2-diphenylpyrrole derivative COX-2 selective inhibitor.

In another embodiment, the invention provides a method of treating a subject with the COX-2 selective inhibitor compound 2-(4-ethoxyphenyl)-4-methyl-1-(4-sulfamoylphenyl)pyrrole the method comprising: measuring the level of PGE2 metabolite in a first biological sample collected from the subject prior to administration of the compound 2-(4-ethoxyphenyl)-4-methyl-1-(4-sulfamoylphenyl)pyrrole, measuring the level of PGE2 metabolite in a second biological sample collected from the subject at least five days after administration of the compound 2-(4-ethoxyphenyl)-4-methyl-1-(4-sulfamoylphenyl)pyrrole, and calculating a ratio by dividing the level of PGE2 metabolite in the second sample by the level of PGE2 metabolite in the first sample, determining that the ratio is lower than about 0.8 and administering to the subject the COX-2 selective inhibitor compound 2-(4-ethoxyphenyl)-4-methyl-1-(4-sulfamoylphenyl)pyrrole.

In another embodiment, the invention provides a method of treating a subject for therapy with a 1,2-diphenylpyrrole derivative COX-2 selective inhibitor compound the method comprising: measuring the level of PGEM in a first biological sample collected from the subject prior to administration of the 1,2-diphenylpyrrole derivative COX-2 selective inhibitor compound, measuring the level of PGEM in a second biological sample collected from the subject at least five days after administration of the 1,2-diphenylpyrrole derivative COX-2 selective inhibitor compound, and calculating a ratio by dividing the level of PGEM in the second sample by the level of PGEM in the first sample, determining that the ratio is lower than about 0.8 and treating the subject with the 1,2-diphenylpyrrole derivative COX-2 selective inhibitor.

In one embodiment, the invention provides a method of selecting a subject for therapy with the COX-2 selective inhibitor compound 2-(4-ethoxyphenyl)-4-methyl-1-(4-sulfamoylphenyl)pyrrole comprising: measuring the level of COX-2 expression in a biological sample collected from the subject prior to administration of the compound, determining that the COX-2 level in the sample is higher than a predetermined value and selecting the subject for therapy with the COX-2 selective inhibitor.

In another embodiment, the invention provides a method of selecting a subject for therapy with a combination comprising the COX-2 selective inhibitor compound 2-(4-ethoxyphenyl)-4-methyl-1-(4-sulfamoylphenyl)pyrrole and a second agent or therapy, the method comprising: measuring the level of COX-2 expression in a biological sample collected from the subject prior to administration of the compound, determining that the COX-2 level in the sample is higher than predetermined value and selecting the subject for therapy with the combination comprising the COX-2 selective inhibitor and the second agent or therapy.

In another embodiment, the invention provides a method of selecting a subject for therapy with a combination comprising the COX-2 selective inhibitor compound 2-(4-ethoxyphenyl)-4-methyl-1-(4-sulfamoylphenyl)pyrrole and a second agent or therapy the method comprising: measuring the level of COX-2 expression in a first biological sample collected from the subject prior to administration of the compound, measuring the level of COX-2 in a second biological sample collected from the subject after administration of the second agent or therapy, and calculating a ratio by dividing the level of COX-2 in the second sample by the level of COX-2 in the first sample, determining that the ratio is higher than a predetermined value and selecting the subject for therapy with the combination of the COX-2 selective inhibitor compound 2-(4-ethoxyphenyl)-4-methyl-1-(4-sulfamoylphenyl)pyrrole and the second agent or therapy.

In yet another embodiment, the invention provides a method of treating a subject with the COX-2 selective inhibitor compound 2-(4-ethoxyphenyl)-4-methyl-1-(4-sulfamoylphenyl)pyrrole comprising: measuring the level of COX-2 expression in a biological sample collected from the subject prior to administration of the compound, determining that the COX-2 level in the sample is higher than a predetermined value and administering to the subject the COX-2 selective inhibitor 2-(4-ethoxyphenyl)-4-methyl-1-(4-sulfamoylphenyl)pyrrole.

In still another embodiment, the invention provides a method of treating a subject with a combination comprising the COX-2 selective inhibitor compound 2-(4-ethoxyphenyl)-4-methyl-1-(4-sulfamoylphenyl)pyrrole and a second agent or therapy, the method comprising: measuring the level of COX-2 expression in a biological sample collected from the subject prior to administration of the compound, determining that the COX-2 level in the sample is higher than a predetermined value and administering to the subject the combination comprising the COX-2 selective inhibitor 2-(4-ethoxyphenyl)-4-methyl-1-(4-sulfamoylphenyl)pyrrole and the second agent or therapy.

In a further embodiment, the invention provides a method of treating a subject with a combination comprising the COX-2 selective inhibitor compound 244-ethoxyphenyl)-4-methyl-1-(4-sulfamoylphenyl)pyrrole and a second agent or therapy the method comprising: measuring the level of COX-2 expression in a first biological sample collected from the subject prior to administration of the compound, measuring the level of COX-2 in a second biological sample collected from the subject after administration of the second agent or therapy, and calculating a ratio by dividing the level of COX-2 in the second sample by the level of COX-2 in the first sample, determining that the ratio is higher than a predetermined value and administering to the subject the combination of the COX-2 selective inhibitor compound 2-(4-ethoxyphenyl)-4-methyl-1-(4-sulfamoylphenyl)pyrrole and the second agent or therapy.

In a still further embodiment, the invention provides a method of selecting a subject for therapy with a 1,2-diphenylpyrrole derivative COX-2 selective inhibitor comprising: measuring the level of COX-2 expression in a biological sample collected from the subject prior to administration of the 1,2-diphenylpyrrole derivative COX-2 selective inhibitor, determining that the COX-2 level in the sample is higher than a predetermined value and selecting the subject for therapy with the 1,2-diphenylpyrrole derivative COX-2 selective inhibitor.

In another embodiment, the invention provides a method of selecting a subject for therapy with a combination comprising a 1,2-diphenylpyrrole derivative COX-2 selective inhibitor and a second agent or therapy, the method comprising: measuring the level of COX-2 expression in a biological sample collected from the subject prior to administration of the 1,2-diphenylpyrrole derivative COX-2 selective inhibitor, determining that the COX-2 level in the sample is higher than predetermined value and selecting the subject for therapy with the combination comprising the COX-2 selective inhibitor and the second agent or therapy 1,2-diphenylpyrrole derivative COX-2 selective inhibitor.

In another embodiment, the invention provides a method of selecting a subject for therapy with a combination comprising a 1,2-diphenylpyrrole derivative COX-2 selective inhibitor and a second agent or therapy the method comprising: measuring the level of COX-2 expression in a first biological sample collected from the subject prior to administration of the 1,2-diphenylpyrrole derivative COX-2 selective inhibitor, measuring the level of COX-2 in a second biological sample collected from the subject after administration of the second agent or therapy, and calculating a ratio by dividing the level of COX-2 in the second sample by the level of COX-2 in the first sample, determining that the ratio is higher than a predetermined value and selecting the subject for therapy with the combination of the 1,2-diphenylpyrrole derivative COX-2 selective inhibitor and the second agent or therapy.

In another embodiment, the invention provides a method of treating a subject with a 1,2-diphenylpyrrole derivative COX-2 selective inhibitor comprising: measuring the level of COX-2 expression in a biological sample collected from the subject prior to administration of the 1,2-diphenylpyrrole derivative COX-2 selective inhibitor, determining that the COX-2 level in the sample is higher than a predetermined value and administering to the subject the 1,2-diphenylpyrrole derivative COX-2 selective inhibitor.

In another embodiment, the invention provides a method of treating a subject with a combination comprising a 1,2-diphenylpyrrole derivative COX-2 selective inhibitor and a second agent or therapy, the method comprising: measuring the level of COX-2 expression in a biological sample collected from the subject prior to administration of the 1,2-diphenylpyrrole derivative COX-2 selective inhibitor, determining that the COX-2 level in the sample is higher than a predetermined value and administering to the subject the combination comprising the COX-2 selective inhibitor and the second agent or therapy.

In another embodiment, the invention provides a method of treating a subject with a combination comprising a 1,2-diphenylpyrrole derivative COX-2 selective inhibitor and a second agent or therapy the method comprising: measuring the level of COX-2 expression in a first biological sample collected from the subject prior to administration of the 1,2-diphenylpyrrole derivative COX-2 selective inhibitor, measuring the level of COX-2 in a second biological sample collected from the subject after administration of the second agent or therapy, and calculating a ratio by dividing the level of COX-2 in the second sample by the level of COX-2 in the first sample, determining that the ratio is higher than a predetermined value and administering to the subject the combination of the 12-diphenylpyrrole derivative COX-2 selective inhibitor and the second agent or therapy.

In another embodiment, the invention provides a method of selecting a subject for therapy with a COX-2 selective inhibitor compound the method comprising: measuring the level of COX-2 expression in a biological sample collected from the subject prior to administration of the compound, determining that the COX-2 level in the sample is higher than about 7 ng/mg for a female subject or higher than about 12 ng/mg for a male subject, and selecting the subject for therapy with the COX-2 selective inhibitor.

In another embodiment, the invention provides a method of selecting a subject for therapy with a 1,2-diphenylpyrrole derivative COX-2 selective inhibitor compound the method comprising: measuring the level of COX-2 expression in a biological sample collected from the subject prior to administration of the compound, determining that the COX-2 level in the sample is higher than about 7 ng/mg for a female subject or higher than about 12 ng/mg for a male subject, and selecting the subject for therapy with the COX-2 selective inhibitor.

In another embodiment, the invention provides a method of selecting a subject for therapy with the COX-2 selective inhibitor compound 2-(4-ethoxyphenyl)-4-methyl-1-(4-sulfamoylphenyl)pyrrole comprising: measuring the level of COX-2 expression in a biological sample collected from the subject prior to administration of the compound, determining that the COX-2 level in the sample is higher than about 7 ng/mg for a female subject or higher than about 12 ng/mg for a male subject, and selecting the subject for therapy with the COX-2 selective inhibitor.

In another embodiment, the invention provides a method of selecting a subject for therapy with a combination of a COX-2 selective inhibitor compound and a second agent or therapy the method comprising: measuring the level of COX-2 expression in a biological sample collected from the subject prior to administration of the compound, determining that the COX-2 level in the sample is higher than about 7 ng/mg for a female subject or higher than about 12 ng/mg for a male subject, and selecting the subject for therapy with the combination comprising the COX-2 selective inhibitor and the second agent or therapy.

In another embodiment, the invention provides a method of selecting a subject for therapy with a combination of a 1,2-diphenylpyrrole derivative COX-2 selective inhibitor compound and a second agent or therapy the method comprising: measuring the level of COX-2 expression in a biological sample collected from the subject prior to administration of the compound, determining that the COX-2 level in the sample is higher than about 7 ng/mg for a female subject or higher than about 12 ng/mg for a male subject, and selecting the subject for therapy with the combination comprising the COX-2 selective inhibitor and the second agent or therapy.

In another embodiment, the invention provides a method of selecting a subject for therapy with a combination comprising the COX-2 selective inhibitor compound 2-(4-ethoxyphenyl)-4-methyl-1-(4-sulfamoylphenyl)pyrrole and a second agent or therapy, the method comprising: measuring the level of COX-2 expression in a biological sample collected from the subject prior to administration of the compound, determining that the COX-2 level in the sample is higher than about 7 ng/mg for a female subject or higher than about 12 ng/mg for a male subject, and selecting the subject for therapy with the combination comprising the COX-2 selective inhibitor and the second agent or therapy.

In another embodiment, the invention provides a method of selecting a subject for therapy with a combination comprising a COX-2 selective inhibitor compound and a second agent or therapy the method comprising: measuring the level of COX-2 expression in a first biological sample collected from the subject prior to administration of the compound, measuring the level of COX-2 in a second biological sample collected from the subject after administration of the second agent or therapy, and calculating a ratio by dividing the level of COX-2 in the second sample by the level of COX-2 in the first sample, determining that the ratio is higher than about 1.2 and selecting the subject for therapy with the combination of the COX-2 selective inhibitor and the second agent or therapy.

In another embodiment, the invention provides a method of selecting a subject for therapy with a combination comprising a 1,2-diphenylpyrrole derivative COX-2 selective inhibitor compound and a second agent or therapy the method comprising: measuring the level of COX-2 expression in a first biological sample collected from the subject prior to administration of the compound, measuring the level of COX-2 in a second biological sample collected from the subject after administration of the second agent or therapy, and calculating a ratio by dividing the level of COX-2 in the second sample by the level of COX-2 in the first sample, determining that the ratio is higher than about 1.2 and selecting the subject for therapy with the combination of the COX-2 selective inhibitor and the second agent or therapy.

In another embodiment, the invention provides a method of selecting a subject for therapy with a combination comprising the COX-2 selective inhibitor compound 2-(4-ethoxyphenyl)-4-methyl-1-(4-sulfamoylphenyl)pyrrole and a second agent or therapy the method comprising: measuring the level of COX-2 expression in a first biological sample collected from the subject prior to administration of the compound, measuring the level of COX-2 in a second biological sample collected from the subject after administration of the second agent or therapy, and calculating a ratio by dividing the level of COX-2 in the second sample by the level of COX-2 in the first sample, determining that the ratio is higher than about 1.2 and selecting the subject for therapy with the combination of the COX-2 selective inhibitor compound 2-(4-ethoxyphenyl)-4-methyl-1-(4-sulfamoylphenyl)pyrrole and the second agent or therapy.

In another embodiment, the invention provides a method of treating a subject with a COX-2 selective inhibitor compound the method comprising: measuring the level of COX-2 expression in a biological sample collected from the subject prior to administration of the compound, determining that the COX-2 level in the sample is higher than about 7 ng/mg for a female subject or higher than about 12 ng/mg for a male subject, and administering to the subject the COX-2 selective inhibitor.

In another embodiment, the invention provides a method of treating a subject with a 1,2-diphenylpyrrole derivative COX-2 selective inhibitor compound the method comprising: measuring the level of COX-2 expression in a biological sample collected from the subject prior to administration of the compound, determining that the COX-2 level in the sample is higher than about 7 ng/mg for a female subject or higher than about 12 ng/mg for a male subject, and administering to the subject the COX-2 selective inhibitor.

In another embodiment, the invention provides a method of treating a subject with the COX-2 selective inhibitor compound 2-(4-ethoxyphenyl)-4-methyl-1-(4-sulfamoylphenyl)pyrrole comprising: measuring the level of COX-2 in a biological sample collected from the subject prior to administration of the compound, determining that the COX-2 expression level in the sample is higher than about 7 ng/mg for a female subject or higher than about 12 ng/mg for a male subject, and administering to the subject the COX-2 selective inhibitor.

In another embodiment, the invention provides a method of treating a subject with a combination of a COX-2 selective inhibitor compound and a second agent or therapy the method comprising: measuring the level of COX-2 expression in a biological sample collected from the subject prior to administration of the compound, determining that the COX-2 level in the sample is higher than about 7 ng/mg for a female subject or higher than about 12 ng/mg for a male subject, and administering to the subject the combination comprising the COX-2 selective inhibitor and the second agent or therapy.

In another embodiment, the invention provides a method of treating a subject with a combination of a 1,2-diphenylpyrrole derivative COX-2 selective inhibitor compound and a second agent or therapy the method comprising: measuring the level of COX-2 expression in a biological sample collected from the subject prior to administration of the compound, determining that the COX-2 level in the sample is higher than about 7 ng/mg for a female subject or higher than about 12 ng/mg for a male subject, and administering to the subject the combination comprising the COX-2 selective inhibitor and the second agent or therapy.

In another embodiment, the invention provides a method of treating a subject with a combination comprising the COX-2 selective inhibitor compound 2-(4-ethoxyphenyl)-4-methyl-1-(4-sulfamoylphenyl)pyrrole and a second agent or therapy, the method comprising: measuring the level of COX-2 expression in a biological sample collected from the subject prior to administration of the compound, determining that the COX-2 level in the sample is higher than about 7 ng/mg for a female subject or higher than about 12 ng/mg for a male subject, and administering to the subject the combination comprising the COX-2 selective inhibitor and the second agent or therapy.

In another embodiment, the invention provides a method of treating a subject with a combination comprising a COX-2 selective inhibitor compound and a second agent or therapy the method comprising: measuring the level of COX-2 expression in a first biological sample collected from the subject prior to administration of the compound, measuring the level of COX-2 in a second biological sample collected from the subject after administration of the second agent or therapy, and calculating a ratio by dividing the level of COX-2 in the second sample by the level of COX-2 in the first sample, determining that the ratio is higher than about 1/and administering to the subject the combination of the COX-2 selective inhibitor and the second agent or therapy.

In another embodiment, the invention provides a method of treating a subject with a combination comprising a 1,2-diphenylpyrrole derivative COX-2 selective inhibitor compound and a second agent or therapy the method comprising: measuring the level of COX-2 expression in a first biological sample collected from the subject prior to administration of the compound, measuring the level of COX-2 in a second biological sample collected from the subject after administration of the second agent or therapy, and calculating a ratio by dividing the level of COX-2 in the second sample by the level of COX-2 in the first sample, determining that the ratio is higher than about 1.2 and administering to the subject the combination of the COX-2 selective inhibitor and the second agent or therapy.

In another embodiment, the invention provides a method of treating a subject with a combination comprising the COX-2 selective inhibitor compound 2-(4-ethoxyphenyl)-4-methyl-1-(4-sulfamoylphenyl)pyrrole and a second agent or therapy the method comprising: measuring the level of COX-2 expression in a first biological sample collected from the subject prior to administration of the compound, measuring the level of COX-2 in a second biological sample collected from the subject after administration of the second agent or therapy, and calculating a ratio by dividing the level of COX-2 in the second sample by the level of COX-2 in the first sample, determining that the ratio is higher than about 1.2 and administering to the subject the combination of the COX-2 selective inhibitor compound 2-(4-ethoxyphenyl)-4-methyl-1-(4-sulfamoylphenyl)pyrrole and the second agent or therapy.

In another embodiment, the invention provides a method of selecting a subject for therapy with the COX-2 selective inhibitor compound 2-(4-ethoxyphenyl)-4-methyl-1-(4-sulfamoylphenyl)pyrrole the method comprising: measuring the level of COX-2 expression in a first biological sample collected from the subject prior to administration of the COX-2 selective inhibitor, measuring the level of COX-2 in a second biological sample collected from the subject at least five days after administration of the selective COX-2 inhibitor, and calculating a ratio by dividing the level of COX-2 in the second sample by the level of COX-2 in the first sample, determining that the ratio is lower than a predetermined value and selecting the subject for therapy with the COX-2 selective inhibitor compound 2-(4-ethoxyphenyl)-4-methyl-1-(4-sulfamoylphenyl)pyrrole.

In another embodiment, the invention provides a method of treating a subject with the COX-2 selective inhibitor compound 2-(4-ethoxyphenyl)-4-methyl-1-(4-sulfamoylphenyl)pyrrole the method comprising: measuring the level of COX-2 expression in a first biological sample collected from the subject prior to administration of the compound, measuring the level of COX-2 in a second biological sample collected from the subject at least five days after administration of the compound, and calculating a ratio by dividing the level of COX-2 in the second sample by the level of COX-2 in the first sample, determining that the ratio is lower than a predetermined value and administering to the subject the COX-2 selective inhibitor compound 2-(4-ethoxyphenyl)-4-methyl-1-(4-sulfamoylphenyl)pyrrole.

In another embodiment, the invention provides a method of selecting a subject for therapy with a 1,2-diphenylpyrrole derivative COX-2 selective inhibitor the method comprising: measuring the level of COX-2 in a first biological sample collected from the subject prior to administration of the compound, measuring the level of COX-2 expression in a second biological sample collected from the subject at least five days after administration of the compound, and calculating a ratio by dividing the level of COX-2 in the second sample by the level of COX-2 in the first sample, determining that the ratio is lower than a predetermined value and selecting the subject for therapy with the 1,2-diphenylpyrrole derivative COX-2 selective inhibitor.

In another embodiment, the invention provides a method of treating a subject with a 1,2-diphenylpyrrole derivative COX-2 selective inhibitor the method comprising: measuring the level of COX-2 expression in a first biological sample collected from the subject prior to administration of the compound, measuring the level of COX-2 in a second biological sample collected from the subject at least after administration of the compound, and calculating a ratio by dividing the level of COX-2 in the second sample by the level of COX-2 in the first sample, determining that the ratio is lower than a predetermined value and administering to the subject the 1,2-diphenylpyrrole derivative COX-2 selective inhibitor.

In another embodiment, the invention provides a method of selecting a subject for therapy with the COX-2 selective inhibitor compound 2-(4-ethoxyphenyl)-4-methyl-1-(4-sulfamoylphenyl)pyrrole the method comprising: measuring the level of COX-2 expression in a first biological sample collected from the subject prior to administration of the compound, measuring the level of COX-2 in a second biological sample collected from the subject at least five days after administration of the compound, and calculating a ratio by dividing the level of COX-2 in the second sample by the level of COX-2 in the first sample, determining that the ratio is lower than about 0.8 and selecting the subject for therapy with the COX-2 selective inhibitor compound 2-(4-ethoxyphenyl)-4-methyl-1-(4-sulfamoylphenyl)pyrrole.

In another embodiment, the invention provides a method of selecting a subject for therapy with a 1,2-diphenylpyrrole derivative COX-2 selective inhibitor compound the method comprising: measuring the level of COX-2 expression in a first biological sample collected from the subject prior to administration of the compound, measuring the level of COX-2 in a second biological sample collected from the subject at least five days after administration of the compound, and calculating a ratio by dividing the level of COX-2 in the second sample by the level of COX-2 in the first sample, determining that the ratio is lower than about 0.8 and selecting the subject for therapy with the COX-2 selective inhibitor.

In another embodiment, the invention provides a method of treating a subject with the COX-2 selective inhibitor compound 2-(4-ethoxyphenyl)-4-methyl-1-(4-sulfamoylphenyl)pyrrole the method comprising: measuring the level of COX-2 expression in a first biological sample collected from the subject prior to administration of the compound, measuring the level of COX-2 in a second biological sample collected from the subject at least five days after administration of the compound, and calculating a ratio by dividing the level of COX-2 in the second sample by the level of COX-2 in the first sample, determining that the ratio is lower than about 0.8 and administering to the subject the COX-2 selective inhibitor compound 2-(4-ethoxyphenyl)-4-methyl-1-(4-sulfamoylphenyl)pyrrole.

In another embodiment, the invention provides a method of treating a subject for therapy with a 1,2-diphenylpyrrole derivative COX-2 selective inhibitor compound the method comprising: measuring the level of COX-2 expression in a first biological sample collected from the subject prior to administration of the compound, measuring the level of COX-2 in a second biological sample collected from the subject at least five days after administration of the compound, and calculating a ratio by dividing the level of COX-2 in the second sample by the level of COX-2 in the first sample, determining that the ratio is lower than about 0.8 and treating the subject with the COX-2 selective inhibitor.

In another embodiment, the invention provides a method of treating a subject with a combination comprising a 1,2-diphenylpyrrole derivative COX-2 selective inhibitor and a second agent or therapy the method comprising: measuring the level of PGEM in a first biological sample collected from the subject prior to administration of the compound, measuring the level of PGEM in a second biological sample collected from the subject after administration of the second agent or therapy, and determining a ratio by dividing the level of PGEM in the second sample by the level of PGEM in the first sample, determining that the ratio is higher than a predetermined value and administering to the subject the combination of the 1,2-diphenylpyrrole derivative COX-2 selective inhibitor and the second agent or therapy wherein administering the combination to the patient reduces resistance to the second agent compared to administering the second agent or treatment alone.

In another embodiment the invention provides a method for selecting a subject for treatment wherein the cancer is bladder cancer and the method further comprising optionally obtaining an additional biological sample from the subject, measuring the level of any of fatty acid binding proteins, annexin V, heat shock protein 27, and lactate dehydrogenase in the biological sample collected from the subject, determining that the level of any of fatty acid binding proteins, annexin V, heat shock protein 27, or lactate dehydrogenase is higher than a predetermined value and selecting the subject for treatment for bladder cancer.

In another embodiment the invention provides a method for selecting a subject for treatment wherein the cancer is breast cancer and the method further comprising optionally obtaining an additional biological sample from the subject, measuring the level of any of lipophilin B, beta-globin, hemopexin and vitamin D-binding protein precursor in the biological sample collected from the subject, determining that the level of any of lipophilin B, beta-globin, hemopexin and vitamin D-binding protein precursor is higher than a predetermined value and selecting the subject for treatment for breast cancer.

In another embodiment the invention provides a method for selecting a subject for treatment wherein the cancer is colorectal cancer and the method further comprising optionally obtaining an additional biological sample from the subject, measuring the level of any of ANXA3, BMP4, LCN2, SPARC, MMP7 and MMP11 in the biological sample collected from the subject, determining that the level of any of ANXA3, BMP4, LCN2, SPARC, MMP7 and MMP11 is higher than a predetermined value and selecting the subject for treatment for colorectal cancer.

In another embodiment the invention provides a method for selecting a subject for treatment wherein the cancer is colorectal cancer and the method further comprising optionally obtaining an additional biological sample from the subject, measuring the level of endocrine cell-expressed protein secretagogin in the biological sample collected from the subject, determining that the level of any of endocrine cell-expressed protein secretagogin is lower than a predetermined value and selecting the subject for treatment for colorectal cancer.

In another embodiment the invention provides a method for selecting a subject for treatment wherein the cancer is esophageal cancer and the method further comprising optionally obtaining an additional biological sample from the subject, measuring the level of periplakin in the biological sample collected from the subject, determining that the level of periplakin is lower than a predetermined value and selecting the subject for treatment for esophageal cancer.

In another embodiment the invention provides a method for selecting a subject for treatment wherein the cancer is gastrointestinal stromal tumor and the method further comprising optionally obtaining an additional biological sample from the subject, measuring the level of any of annexin V, high mobility group protein 1, C13orf2, glutamate dehydrogenase 1, and fibrinogen beta chain in the biological sample collected from the subject, determining that the level of any of annexin V, high mobility group protein 1, C13orf2, glutamate dehydrogenase 1, and fibrinogen beta chain is higher than a predetermined value and selecting the subject for treatment for gastrointestinal stromal tumor.

In another embodiment the invention provides a method for selecting a subject for treatment wherein the cancer is gastrointestinal stromal tumor and the method further comprising optionally obtaining an additional biological sample from the subject, measuring the level of RoXaN in the biological sample collected from the subject, determining that the level of RoXaN is lower than a predetermined value and selecting the subject for treatment for gastrointestinal stromal tumor.

In another embodiment the invention provides a method for selecting a subject for treatment wherein the cancer is hepatocellular carcinoma and the method further comprising optionally obtaining an additional biological sample from the subject, measuring the level of any of pro-apolipoprotein, alpha2-HS glycoprotein, apolipoprotein A-IV precursor, 14-3-3-gamma protein, complement C3a and PRO1708/PRO2044 in the biological sample collected from the subject, determining that the level of any of pro-apolipoprotein, alpha2-HS glycoprotein, apolipoprotein A-IV precursor, 14-3-3-gamma protein, complement C3a and PRO1708/PRO2044 is higher than a predetermined value and selecting the subject for treatment for hepatocellular carcinoma.

In another embodiment the invention provides a method for selecting a subject for treatment wherein the cancer is hepatocellular carcinoma and the method further comprising optionally obtaining an additional biological sample from the subject, measuring the level of any of alpha2-glycoprotein, complement C3a and alpha1-antitrypsin in the biological sample collected from the subject, determining that the level of any of alpha2-glycoprotein, complement C3a and alpha1-antitrypsin is lower than a predetermined value and selecting the subject for treatment for hepatocellular carcinoma.

In another embodiment the invention provides a method for selecting a subject for treatment wherein the cancer is lung adenocarcinoma and the method further comprising optionally obtaining an additional biological sample from the subject, measuring the level of any of immunoglobulin lambda chain, transthyretin monomer, haptoglobin-alfa 2a and serum amyloid protein in the biological sample collected from the subject, determining that the level of any of immunoglobulin lambda chain, transthyretin monomer, haptoglobin-alfa 2a and serum amyloid protein is higher than a predetermined value and selecting the subject for treatment for lung adenocarcinoma.

In another embodiment the invention provides a method for selecting a subject for treatment wherein the cancer is lung adenocarcinoma and the method further comprising optionally obtaining an additional biological sample from the subject, measuring the level of apolipoprotein A-I in the biological sample collected from the subject, determining that the level of apolipoprotein A-I is lower than a predetermined value and selecting the subject for treatment for lung adenocarcinoma.

In another embodiment the invention provides a method for selecting a subject for treatment wherein the cancer is follicular lymphoma and the method further comprising optionally obtaining an additional biological sample from the subject, measuring the level of histone H4 in the biological sample collected from the subject, determining that the level of histone H4 is higher than a predetermined value and selecting the subject for treatment for follicular lymphoma.

In another embodiment the invention provides a method for selecting a subject for treatment wherein the cancer is follicular lymphoma and the method further comprising optionally obtaining an additional biological sample from the subject, measuring the level of histone H4 in the biological sample collected from the subject, determining that the level of histone H4 is lower than a predetermined value and selecting the subject for treatment for follicular lymphoma.

In another embodiment the invention provides a method for selecting a subject for treatment wherein the cancer is nasopharyngeal carcinoma and the method further comprising optionally obtaining an additional biological sample from the subject, measuring the level of any of serum amyloid A and ceruloplasmin in the biological sample collected from the subject, determining that the level of any of serum amyloid A and ceruloplasmin is higher than a predetermined value and selecting the subject for treatment for nasopharyngeal carcinoma.

In another embodiment the invention provides a method for selecting a subject for treatment wherein the cancer is ovarian cancer and the method further comprising optionally obtaining an additional biological sample from the subject, measuring the level of any of CA-125 and haptoglobin precursor in the biological sample collected from the subject, determining that the level of any of CA-125 and haptoglobin precursor is higher than a predetermined value and selecting the subject for treatment for ovarian cancer.

In another embodiment the invention provides a method for selecting a subject for treatment wherein the cancer is ovarian cancer and the method further comprising optionally obtaining an additional biological sample from the subject, measuring the level of transferrin precursor in the biological sample collected from the subject, determining that the level of transferrin precursor is lower than a predetermined value and selecting the subject for treatment for ovarian cancer.

In another embodiment the invention provides a method for selecting a subject for treatment wherein the cancer is pancreatic cancer and the method further comprising optionally obtaining an additional biological sample from the subject, measuring the level of fibrinogen gamma in the biological sample collected from the subject, determining that the level of fibrinogen gamma is higher than a predetermined value and selecting the subject for treatment for pancreatic cancer.

In another embodiment the invention provides a method for selecting a subject for treatment wherein the cancer is prostate cancer and the method further comprising optionally obtaining an additional biological sample from the subject, measuring the level of annexin I in the biological sample collected from the subject, determining that the level of annexin I is lower than a predetermined value and selecting the subject for treatment for prostate cancer.

In another embodiment the invention provides a method for selecting a subject for treatment wherein the cancer is urothelial carcinoma and the method further comprising optionally obtaining an additional biological sample from the subject, measuring the level of fibrinopeptide A in the biological sample collected from the subject, determining that the level of fibrinopeptide A is higher than a predetermined value and selecting the subject for treatment for urothelial carcinoma.

In another embodiment the invention provides a method for selecting a subject for treatment wherein the cancer is urothelial carcinoma and the method further comprising optionally obtaining an additional biological sample from the subject, measuring the level of fibrinopeptide A in the biological sample collected from the subject, determining that the level of fibrinopeptide A is lower than a predetermined value and selecting the subject for treatment for urothelial carcinoma.

In another embodiment the invention provides a method for selecting a subject for treatment wherein the cancer is renal cell carcinoma and the method further comprising optionally obtaining an additional biological sample from the subject, measuring the level of sorbitol in the biological sample collected from the subject, determining that the level of sorbitol is higher than a predetermined value and selecting the subject for treatment for renal cell carcinoma.

In another embodiment the invention provides a method for selecting a subject for treatment wherein the cancer is metastatic breast cancer and the method further comprising optionally obtaining an additional biological sample from the subject, measuring the level of alpha-actinin in the biological sample collected from the subject, determining that the level of alpha-actinin is higher than a predetermined value and selecting the subject for treatment for metastatic breast cancer.

In another embodiment the invention provides a method for selecting a subject for treatment wherein the cancer is prostate cancer and the method further comprising optionally obtaining an additional biological sample from the subject, measuring the level of alpha-actinin in the biological sample collected from the subject, determining that the level of alpha-actinin is higher than a predetermined value and selecting the subject for treatment for prostate cancer.

In another embodiment the invention provides a method for selecting a subject for treatment wherein the cancer is skin cancer and the method further comprising optionally obtaining an additional biological sample from the subject, measuring the level of alpha-actinin in the biological sample collected from the subject, determining that the level of alpha-actinin is higher than a predetermined value and selecting the subject for treatment for skin cancer.

In another embodiment the invention provides a method for selecting a subject for treatment wherein the cancer is breast cancer and the method further comprising optionally obtaining an additional biological sample from the subject, measuring the level of HER-2 in the biological sample collected from the subject, determining that the level of HER-2 is higher than a predetermined value and selecting the subject for treatment for breast cancer.

In another embodiment the invention provides a method for selecting a subject for treatment wherein the cancer is breast cancer and the method further comprising optionally obtaining an additional biological sample from the subject, measuring the level of estrogen receptor in the biological sample collected from the subject, determining that the level of estrogen receptor is higher than a predetermined value and selecting the subject for treatment for breast cancer.

In another embodiment the invention provides a method for selecting a subject for treatment wherein the cancer is hepatocellular carcinoma and the method further comprising optionally obtaining an additional biological sample from the subject, measuring the level of any of Hsp27, Hsp70, glucose-regulated protein 78 and Hcc-2 in the biological sample collected from the subject, determining that the level of any of Hsp27, Hsp70, glucose-regulated protein 78 and Hcc-2 is higher than a predetermined value and selecting the subject for treatment for hepatocellular carcinoma.

In another embodiment the invention provides a method for selecting a subject for treatment wherein the cancer is bladder cancer and the method further comprising optionally obtaining an additional biological sample from the subject, measuring the level of urinary nuclear matrix protein NMP22 in the biological sample collected from the subject, determining that the level of urinary nuclear matrix protein NMP22 is higher than a predetermined value and selecting the subject for treatment for bladder cancer.

In another embodiment the invention provides a method for selecting a subject for treatment wherein the cancer is ER(+)-breast cancer and the method further comprising optionally obtaining an additional biological sample from the subject, measuring the level of any of CRABP-II, cyclophilin A, neudesin and hemoglobin in the biological sample collected from the subject, determining that the level of any of CRABP-II, cyclophilin A, neudesin and hemoglobin is higher than a predetermined value and selecting the subject for tamoxifen treatment for ER(+)-breast cancer.

In another embodiment the invention provides a method for selecting a subject for treatment wherein the cancer is ER(+)-breast cancer and the method further comprising optionally obtaining an additional biological sample from the subject, measuring the level of any of cytochrome b5 and transgelin in the biological sample collected from the subject, determining that the level of any of cytochrome b5 and transgelin is lower than a predetermined value and selecting the subject for tamoxifen treatment for ER(+)-breast cancer.

In another embodiment the invention provides a method for selecting a subject for treatment wherein the cancer is B cell chronic lymphocytic leukemia and the method further comprising optionally obtaining an additional biological sample from the subject, measuring the level of Hsp27 in the biological sample collected from the subject, determining that the level of Hsp27 is higher than a predetermined value and selecting the subject for treatment for B cell chronic lymphocytic leukemia.

In another embodiment the invention provides a method for selecting a subject for treatment wherein the cancer is B cell chronic lymphocytic leukemia and the method further comprising optionally obtaining an additional biological sample from the subject, measuring the level of any of thioredoxin peroxidase 2 and protein disulfide isomerase in the biological sample collected from the subject, determining that the level of any of thioredoxin peroxidase 2 and protein disulfide isomerase is lower than a predetermined value and selecting the subject for treatment for B cell chronic lymphocytic leukemia.

In another embodiment the invention provides a method for selecting a subject for treatment wherein the cancer is prostate cancer and the method further comprising optionally obtaining an additional biological sample from the subject, measuring the level of prostate-specific antigen in the biological sample collected from the subject, determining that the level of prostate-specific antigen is higher than a predetermined value and selecting the subject for treatment for prostate cancer.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The following drawings are part of the present specification and are included to further demonstrate certain aspects of what is presently disclosed. The claimed embodiments may be better understood by reference to one or more of these drawings in combination with the detailed description of the specific embodiments presented herein.

FIG. 1 is schematic diagram depicting the production and metabolism of prostaglandin E₂.

### DETAILED DESCRIPTION OF THE INVENTION

The present invention is based, in part, on the discovery of a method for enhancing the treatment of a subject having a condition wherein expression of COX-2 plays a role. The inventors have discovered that by determining the level of a PGE2 metabolite (e.g., PGEM) in a sample obtained from a patient a therapeutic regimen including administration of a COX-2 selective inhibitor can be tailored such that therapeutic objectives are achieved while minimizing one or more side effects generally associated with treatments with COX-2 inhibitors.

The inventors have uncovered base line levels of metabolites that allow for establishing an effective therapeutic regimen to increase positive outcomes a selected patient. The invention provides effective therapeutic regimens involving one or more COX-2 inhibitors and a second therapeutic agent or treatment. By determining the effect of the second therapeutic agent or treatment on COX-2 expression for a subject, the invention provides therapeutic methods wherein one or more COX-2 inhibitors are administered in combination with the second therapeutic agent or therapy resulting in enhanced outcomes for the subject.

The invention provides methods for treatment with a combination comprising a COX-2 selective inhibitor and a second agent or therapy, wherein administering the combination to the patient provides an enhanced treatment compared to administering the COX-2 selective inhibitor alone or the second agent or treatment alone.

The methods of the invention are also advantageous in that they provide methods for treatment with a combination comprising a COX-2 selective inhibitor and a second agent or therapy wherein the combination is administered at a lower dosage compared to administering the COX-2 selective inhibitor alone or the second agent or treatment alone.

More over, the methods of the invention are advantageous in that the invention provides methods for treatment with a combination comprising a COX-2 selective inhibitor and a second agent or therapy wherein administering the combination to the patient reduces side effects compared to administering the COX-2 selective inhibitor alone or the second agent or treatment alone.

Additional advantages of the invention include providing methods for treatment with a combination comprising a COX-2 selective inhibitor and a second agent or therapy wherein administering the combination to the patient reduces resistance to the second agent compared to administering the second agent or treatment alone.

In one embodiment, the invention provides a method of assessing response to treatment in a subject undergoing therapy with a 1,2-diphenylpyrrole derivative COX-2 selective inhibitor the method comprising: measuring the PGEM level in the subjects urine at least a first and a second point in time to produce at least a first level of PGEM and a second level of PGEM, wherein the at least first and second points in time are separated from each other by an interval of at least about five days, and wherein a decrease in the second level of PGEM compared to the first level of PGEM is indicative of positive response to treatment.

In another embodiment, the invention provides a method of treating a subject with a combination comprising the COX-2 selective inhibitor compound 2-(4-ethoxyphenyl)-4-methyl-1-(4-sulfamoylphenyl)pyrrole and a second agent or therapy, the method comprising: measuring the level of PGEM in a biological sample collected from the subject prior to administration of the compound, determining that the PGEM level in the sample is higher than a predetermined value and administering to the subject the COX-2 selective inhibitor; wherein administering the combination to the patient provides an enhanced treatment compared to administering the COX-2 selective inhibitor alone or the second agent or treatment alone.

In another embodiment, the invention provides a method of treating a subject with a combination comprising the COX-2 selective inhibitor compound 2-(4-ethoxyphenyl)-4-methyl-1-(4-sulfamoylphenyl)pyrrole and a second agent or therapy, the method comprising: measuring the level of PGEM in a biological sample collected from the subject prior to administration of the compound, determining that the PGEM level in the sample is higher than a predetermined value and administering to the subject the COX-2 selective inhibitor wherein the combination is administered at a lower dosage compared to administering the COX-2 selective inhibitor alone or the second agent or treatment alone.

In another embodiment, the invention provides a method of treating a subject with a combination comprising the COX-2 selective inhibitor compound 2-(4-ethoxyphenyl)-4-methyl-1-(4-sulfamoylphenyl)pyrrole and a second agent or therapy, the method comprising: measuring the level of PGEM in a biological sample collected from the subject prior to administration of the compound, determining that the PGEM level in the sample is higher than a predetermined value and administering to the subject the COX-2 selective inhibitor wherein administering the combination to the patient reduces side effects compared to administering the COX-2 selective inhibitor alone or the second agent or treatment alone.

In another embodiment, the invention provides a method of treating a subject with a combination comprising the COX-2 selective inhibitor compound 2-(4-ethoxyphenyl)-4-methyl-1-(4-sulfamoylphenyl)pyrrole and a second agent or therapy, the method comprising: measuring the level of PGEM in a biological sample collected from the subject prior to administration of the compound, determining that the PGEM level in the sample is higher than a predetermined value and administering to the subject the COX-2 selective inhibitor wherein administering the combination to the patient reduces resistance to the second agent compared to administering the second agent or treatment alone.

In another embodiment, the invention provides a method of selecting a subject for therapy with a combination comprising the COX-2 selective inhibitor compound 2-(4-ethoxyphenyl)-4-methyl-1-(4-sulfamoylphenyl)pyrrole and a second agent or therapy the method comprising: measuring the level of PGEM in a first biological sample collected from the subject prior to administration of the compound, measuring the level of PGEM in a second biological sample collected from the subject after administration of the second agent or therapy, and determining a ratio by dividing the level of PGEM in the second sample by the level of PGEM in the first sample, determining that the ratio is higher than a predetermined value and selecting the subject for therapy with the combination of the COX-2 selective inhibitor compound 2-(4-ethoxyphenyl)-4-methyl-1-(4-sulfamoylphenyl)pyrrole and the second agent or therapy; wherein administering the combination to the patient provides an enhanced treatment compared to administering the COX-2 selective inhibitor alone or the second agent or treatment alone.

In another embodiment, the invention provides a method of selecting a subject for therapy with a combination comprising the COX-2 selective inhibitor compound 2-(4-ethoxyphenyl)-4-methyl-1-(4-sulfamoylphenyl)pyrrole and a second agent or therapy the method comprising: measuring the level of PGEM in a first biological sample collected from the subject prior to administration of the compound, measuring the level of PGEM in a second biological sample collected from the subject after administration of the second agent or therapy, and determining a ratio by dividing the level of PGEM in the second sample by the level of PGEM in the first sample, determining that the ratio is higher than a predetermined value and selecting the subject for therapy with the combination of the COX-2 selective inhibitor compound 2-(4-ethoxyphenyl)-4-methyl-1-(4-sulfamoylphenyl)pyrrole and the second agent or therapy wherein the combination is administered at a lower dosage compared to administering the COX-2 selective inhibitor alone or the second agent or treatment alone.

In another embodiment, the invention provides a method of selecting a subject for therapy with a combination comprising the COX-2 selective inhibitor compound 2-(4-ethoxyphenyl)-4-methyl-1-(4-sulfamoylphenyl)pyrrole and a second agent or therapy the method comprising: measuring the level of PGEM in a first biological sample collected from the subject prior to administration of the compound, measuring the level of PGEM in a second biological sample collected from the subject after administration of the second agent or therapy, and determining a ratio by dividing the level of PGEM in the second sample by the level of PGEM in the first sample, determining that the ratio is higher than a predetermined value and selecting the subject for therapy with the combination of the COX-2 selective inhibitor compound 2-(4-ethoxyphenyl)-4-methyl-1-(4-sulfamoylphenyl)pyrrole and the second agent or therapy wherein administering the combination to the patient reduces side effects compared to administering the COX-2 selective inhibitor alone or the second agent or treatment alone.

In another embodiment, the invention provides a method of selecting a subject for therapy with a combination comprising the COX-2 selective inhibitor compound 2-(4-ethoxyphenyl)-4-methyl-1-(4-sulfamoylphenyl)pyrrole and a second agent or therapy the method comprising: measuring the level of PGEM in a first biological sample collected from the subject prior to administration of the compound, measuring the level of PGEM in a second biological sample collected from the subject after administration of the second agent or therapy, and determining a ratio by dividing the level of PGEM in the second sample by the level of PGEM in the first sample, determining that the ratio is higher than a predetermined value and selecting the subject for therapy with the combination of the COX-2 selective inhibitor compound 2-(4-ethoxyphenyl)-4-methyl-1-(4-sulfamoylphenyl)pyrrole and the second agent or therapy wherein administering the combination to the patient reduces resistance to the second agent compared to administering the second agent or treatment alone.

In another embodiment, the invention provides a method of treating a subject with a combination comprising a 1,2-diphenylpyrrole derivative COX-2 selective inhibitor and a second agent or therapy, the method comprising: measuring the level of PGEM in a biological sample collected from the subject prior to administration of the compound, determining that the PGEM level in the sample is higher than a predetermined value and administering to the subject the 1,2-diphenylpyrrole derivative COX-2 selective inhibitor; wherein administering the combination to the patient provides an enhanced treatment compared to administering the COX-2 selective inhibitor alone or the second agent or treatment alone.

In another embodiment, the invention provides a method of treating a subject with a combination comprising a 1,2-diphenylpyrrole derivative COX-2 selective inhibitor and a second agent or therapy, the method comprising: measuring the level of PGEM in a biological sample collected from the subject prior to administration of the compound, determining that the PGEM level in the sample is higher than a predetermined value and administering to the subject the 1,2-diphenylpyrrole derivative COX-2 selective inhibitor wherein the combination is administered at a lower dosage compared to administering the COX-2 selective inhibitor alone or the second agent or treatment alone.

In another embodiment, the invention provides a method of treating a subject with a combination comprising a 1,2-diphenylpyrrole derivative COX-2 selective inhibitor and a second agent or therapy, the method comprising: measuring the level of PGEM in a biological sample collected from the subject prior to administration of the compound, determining that the PGEM level in the sample is higher than a predetermined value and administering to the subject the 1,2-diphenylpyrrole derivative COX-2 selective inhibitor wherein administering the combination to the patient reduces side effects compared to administering the COX-2 selective inhibitor alone or the second agent or treatment alone.

In another embodiment, the invention provides a method of treating a subject with a combination comprising a 1,2-diphenylpyrrole derivative COX-2 selective inhibitor and a second agent or therapy, the method comprising: measuring the level of PGEM in a biological sample collected from the subject prior to administration of the compound, determining that the PGEM level in the sample is higher than a predetermined value and administering to the subject the 1,2-diphenylpyrrole derivative COX-2 selective inhibitor wherein administering the combination to the patient reduces resistance to the second agent compared to administering the second agent or treatment alone.

In another embodiment, the invention provides a method of selecting a subject for therapy with a combination comprising a 1,2-diphenylpyrrole derivative COX-2 selective inhibitor and a second agent or therapy the method comprising: measuring the level of PGEM in a first biological sample collected from the subject prior to administration of the compound, measuring the level of PGEM in a second biological sample collected from the subject after administration of the second agent or therapy, and determining a ratio by dividing the level of PGEM in the second sample by the level of PGEM in the first sample, determining that the ratio is higher than a predetermined value and selecting the subject for therapy with the combination of the 1,2-diphenylpyrrole derivative COX-2 selective inhibitor and the second agent or therapy; wherein administering the combination to the patient provides an enhanced treatment compared to administering the COX-2 selective inhibitor alone or the second agent or treatment alone.

In another embodiment, the invention provides a method of selecting a subject for therapy with a combination comprising a 1,2-diphenylpyrrole derivative COX-2 selective inhibitor and a second agent or therapy the method comprising: measuring the level of PGEM in a first biological sample collected from the subject prior to administration of the compound, measuring the level of PGEM in a second biological sample collected from the subject after administration of the second agent or therapy, and determining a ratio by dividing the level of PGEM in the second sample by the level of PGEM in the first sample, determining that the ratio is higher than a predetermined value and selecting the subject for therapy with the combination of the 1,2-diphenylpyrrole derivative COX-2 selective inhibitor and the second agent or therapy wherein the combination is administered at a lower dosage compared to administering the COX-2 selective inhibitor alone or the second agent or treatment alone.

In another embodiment, the invention provides a method of selecting a subject for therapy with a combination comprising a 1,2-diphenylpyrrole derivative COX-2 selective inhibitor and a second agent or therapy the method comprising: measuring the level of PGEM in a first biological sample collected from the subject prior to administration of the compound, measuring the level of PGEM in a second biological sample collected from the subject after administration of the second agent or therapy, and determining a ratio by dividing the level of PGEM in the second sample by the level of PGEM in the first sample, determining that the ratio is higher than a predetermined value and selecting the subject for therapy with the combination of the 1,2-diphenylpyrrole derivative COX-2 selective inhibitor and the second agent or therapy wherein administering the combination to the patient reduces side effects compared to administering the COX-2 selective inhibitor alone or the second agent or treatment alone.

In another embodiment, the invention provides a method of treating a subject with a combination comprising a 1,2-diphenylpyrrole derivative COX-2 selective inhibitor and a second agent or therapy the method comprising: measuring the level of PGEM in a first biological sample collected from the subject prior to administration of the compound, measuring the level of PGEM in a second biological sample collected from the subject after administration of the second agent or therapy, and determining a ratio by dividing the level of PGEM in the second sample by the level of PGEM in the first sample, determining that the ratio is higher than a predetermined value and administering to the subject the combination of the 1,2-diphenylpyrrole derivative COX-2 selective inhibitor and the second agent or therapy wherein administering the combination to the patient reduces resistance to the second agent compared to administering the second agent or treatment alone.

In a further embodiment, the invention provides a method of treatment with a combination comprising a COX-2 selective inhibitor, at least one or more additional agents or therapies; and/or an optional third, or additional, or subsequent agent or therapy that is administered with the combination. In one embodiment, the invention provides a method for treating a subject with the combination comprising a COX-2 selective inhibitor compound and at least one or more additional agents or therapies; and/or an optional third, or additional, or subsequent agent or therapy wherein the combination and/or optional third, or additional, or subsequent agent or therapy are administered concurrently. In another embodiment, the invention provides a method for treating a subject with the combination comprising a COX-2 selective inhibitor compound and at least one or more additional agents or therapies; and/or an optional third, or additional, or subsequent agent or therapy wherein the combination and/or optional third, or additional, or subsequent agent or therapy are administered sequentially. In another embodiment, the invention provides a method for treating a subject with a combination comprising a COX-2 selective inhibitor compound and at least one or more additional agents or therapies; and/or an optional third, or additional, or subsequent agent or therapy wherein the additional or subsequent agent or therapy comprises at least between 1 and 4 additional agents or therapies.

The present disclosure generally relates to a method for screening patients to determine which patients will be responders to a proposed medical treatment or therapy for a given disorder. In one aspect, the disclosure relates to a method for screening patients who are candidates for COX-2 inhibitor therapy for the treatment of cancer, pain, inflammation, and/or related disorders. The screening process reliably and significantly improves the predictability of responsiveness to COX-2 inhibitor therapy as well as survival outcomes in candidate patients based on prognostic factors and data known for and/or elicited from a patient prior to treatment. The present disclosure also provides a method for selecting patients for treatment of cancer, inflammation, pain, and/or related disorders with a COX-2 inhibitor based upon the patients' prostaglandin metabolite profile.

Also provided herein are methods for screening patients who are candidates for a combination of a COX-2 inhibitor and a second therapeutic agent treatment of cancer, pain, inflammation, and/or related disorders. In one aspect, the disclosure relates to a method for screening patients who are candidates for COX-2 inhibitor therapy for the treatment of cancer, pain, inflammation, and/or related disorders based upon the patients' PGE-M profile.

Also provided herein are methods for selecting a patient for treatment with 2-(4-ethoxyphenyl)-4-methyl-1-(4-sulfamoylphenyl)pyrrole provided a determined PGE-M level from a patient sample is above a pre-determined PGE-M level. Provided herein, are methods for selecting a patient for treatment with a combination of 2-4-(ethoxyphenyl)-4-methyl-1-(4-sulfamoylphenyl)pyrrole and a second therapeutic agent provided a determined PGE-M level from a patient sample is above a pre-determined PGE-M level.

In another embodiment the invention provides a method for selecting a subject for treatment wherein the cancer is bladder cancer and the method further comprising optionally obtaining an additional biological sample from the subject, measuring the level of any of fatty acid binding proteins, annexin V, heat shock protein 27, and lactate dehydrogenase in the biological sample collected from the subject, determining that the level of any of fatty acid binding proteins, annexin V, heat shock protein 27, or lactate dehydrogenase is higher than a predetermined value and selecting the subject for treatment for bladder cancer.

In another embodiment the invention provides a method for selecting a subject for treatment wherein the cancer is breast cancer and the method further comprising optionally obtaining an additional biological sample from the subject, measuring the level of any of lipophilin B, beta-globin, hemopexin and vitamin D-binding protein precursor in the biological sample collected from the subject, determining that the level of any of lipophilin B, beta-globin, hemopexin and vitamin D-binding protein precursor is higher than a predetermined value and selecting the subject for treatment for breast cancer.

In another embodiment the invention provides a method for selecting a subject for treatment wherein the cancer is colorectal cancer and the method further comprising optionally obtaining an additional biological sample from the subject, measuring the level of any of ANXA3, BMP4, LCN2, SPARC, MMP7 and MMP11 in the biological sample collected from the subject, determining that the level of any of ANXA3, BMP4, LCN2, SPARC, MMP7 and MMP11 is higher than a predetermined value and selecting the subject for treatment for colorectal cancer.

In another embodiment the invention provides a method for selecting a subject for treatment wherein the cancer is colorectal cancer and the method further comprising optionally obtaining an additional biological sample from the subject, measuring the level of endocrine cell-expressed protein secretagogin in the biological sample collected from the subject, determining that the level of any of endocrine cell-expressed protein secretagogin is lower than a predetermined value and selecting the subject for treatment for colorectal cancer.

In another embodiment the invention provides a method for selecting a subject for treatment wherein the cancer is esophageal cancer and the method further comprising optionally obtaining an additional biological sample from the subject, measuring the level of periplakin in the biological sample collected from the subject, determining that the level of periplakin is lower than a predetermined value and selecting the subject for treatment for esophageal cancer.

In another embodiment the invention provides a method for selecting a subject for treatment wherein the cancer is gastrointestinal stromal tumor and the method further comprising optionally obtaining an additional biological sample from the subject, measuring the level of any of annexin V, high mobility group protein 1, C13orf2, glutamate dehydrogenase 1, and fibrinogen beta chain in the biological sample collected from the subject, determining that the level of any of annexin V, high mobility group protein 1, C13orf2, glutamate dehydrogenase 1, and fibrinogen beta chain is higher than a predetermined value and selecting the subject for treatment for gastrointestinal stromal tumor.

In another embodiment the invention provides a method for selecting a subject for treatment wherein the cancer is gastrointestinal stromal tumor and the method further comprising optionally obtaining an additional biological sample from the subject, measuring the level of RoXaN in the biological sample collected from the subject, determining that the level of RoXaN is lower than a predetermined value and selecting the subject for treatment for gastrointestinal stromal tumor.

In another embodiment the invention provides a method for selecting a subject for treatment wherein the cancer is hepatocellular carcinoma and the method further comprising optionally obtaining an additional biological sample from the subject, measuring the level of any of pro-apolipoprotein, alpha2-HS glycoprotein, apolipoprotein A-IV precursor, 14-3-3-gamma protein, complement C3a and PRO1708/PRO2044 in the biological sample collected from the subject, determining that the level of any of pro-apolipoprotein, alpha2-HS glycoprotein, apolipoprotein A-IV precursor, 14-3-3-gamma protein, complement C3a and PRO1708/PRO2044 is higher than a predetermined value and selecting the subject for treatment for hepatocellular carcinoma.

In another embodiment the invention provides a method for selecting a subject for treatment wherein the cancer is hepatocellular carcinoma and the method further comprising optionally obtaining an additional biological sample from the subject, measuring the level of any of alpha2-glycoprotein, complement C3a and alpha1-antitrypsin in the biological sample collected from the subject, determining that the level of any of alpha2-glycoprotein, complement C3a and alpha1-antitrypsin is lower than a predetermined value and selecting the subject for treatment for hepatocellular carcinoma.

In another embodiment the invention provides a method for selecting a subject for treatment wherein the cancer is lung adenocarcinoma and the method further comprising optionally obtaining an additional biological sample from the subject, measuring the level of any of immunoglobulin lambda chain, transthyretin monomer, haptoglobin-alfa 2a and serum amyloid protein in the biological sample collected from the subject, determining that the level of any of immunoglobulin lambda chain, transthyretin monomer, haptoglobin-alfa 2a and serum amyloid protein is higher than a predetermined value and selecting the subject for treatment for lung adenocarcinoma.

In another embodiment the invention provides a method for selecting a subject for treatment wherein the cancer is lung adenocarcinoma and the method further comprising optionally obtaining an additional biological sample from the subject, measuring the level of apolipoprotein A-I in the biological sample collected from the subject, determining that the level of apolipoprotein A-I is lower than a predetermined value and selecting the subject for treatment for lung adenocarcinoma.

In another embodiment the invention provides a method for selecting a subject for treatment wherein the cancer is follicular lymphoma and the method further comprising optionally obtaining an additional biological sample from the subject, measuring the level of histone H4 in the biological sample collected from the subject, determining that the level of histone H4 is higher than a predetermined value and selecting the subject for treatment for follicular lymphoma.

In another embodiment the invention provides a method for selecting a subject for treatment wherein the cancer is follicular lymphoma and the method further comprising optionally obtaining an additional biological sample from the subject, measuring the level of histone H4 in the biological sample collected from the subject, determining that the level of histone H4 is lower than a predetermined value and selecting the subject for treatment for follicular lymphoma.

In another embodiment the invention provides a method for selecting a subject for treatment wherein the cancer is nasopharyngeal carcinoma and the method further comprising optionally obtaining an additional biological sample from the subject, measuring the level of any of serum amyloid A and ceruloplasmin in the biological sample collected from the subject, determining that the level of any of serum amyloid A and ceruloplasmin is higher than a predetermined value and selecting the subject for treatment for nasopharyngeal carcinoma.

In another embodiment the invention provides a method for selecting a subject for treatment wherein the cancer is ovarian cancer and the method further comprising optionally obtaining an additional biological sample from the subject, measuring the level of any of CA-125 and haptoglobin precursor in the biological sample collected from the subject, determining that the level of any of CA-125 and haptoglobin precursor is higher than a predetermined value and selecting the subject for treatment for ovarian cancer.

In another embodiment the invention provides a method for selecting a subject for treatment wherein the cancer is ovarian cancer and the method further comprising optionally obtaining an additional biological sample from the subject, measuring the level of transferrin precursor in the biological sample collected from the subject, determining that the level of transferrin precursor is lower than a predetermined value and selecting the subject for treatment for ovarian cancer.

In another embodiment the invention provides a method for selecting a subject for treatment wherein the cancer is pancreatic cancer and the method further comprising optionally obtaining an additional biological sample from the subject, measuring the level of fibrinogen gamma in the biological sample collected from the subject, determining that the level of fibrinogen gamma is higher than a predetermined value and selecting the subject for treatment for pancreatic cancer.

In another embodiment the invention provides a method for selecting a subject for treatment wherein the cancer is prostate cancer and the method further comprising optionally obtaining an additional biological sample from the subject, measuring the level of annexin I in the biological sample collected from the subject, determining that the level of annexin I is lower than a predetermined value and selecting the subject for treatment for prostate cancer.

In another embodiment the invention provides a method for selecting a subject for treatment wherein the cancer is urothelial carcinoma and the method further comprising optionally obtaining an additional biological sample from the subject, measuring the level of fibrinopeptide A in the biological sample collected from the subject, determining that the level of fibrinopeptide A is higher than a predetermined value and selecting the subject for treatment for urothelial carcinoma.

In another embodiment the invention provides a method for selecting a subject for treatment wherein the cancer is urothelial carcinoma and the method further comprising optionally obtaining an additional biological sample from the subject, measuring the level of fibrinopeptide A in the biological sample collected from the subject, determining that the level of fibrinopeptide A is lower than a predetermined value and selecting the subject for treatment for urothelial carcinoma.

In another embodiment the invention provides a method for selecting a subject for treatment wherein the cancer is renal cell carcinoma and the method further comprising optionally obtaining an additional biological sample from the subject, measuring the level of sorbitol in the biological sample collected from the subject, determining that the level of sorbitol is higher than a predetermined value and selecting the subject for treatment for renal cell carcinoma.

In another embodiment the invention provides a method for selecting a subject for treatment wherein the cancer is metastatic breast cancer and the method further comprising optionally obtaining an additional biological sample from the subject, measuring the level of alpha-actinin in the biological sample collected from the subject, determining that the level of alpha-actinin is higher than a predetermined value and selecting the subject for treatment for metastatic breast cancer.

In another embodiment the invention provides a method for selecting a subject for treatment wherein the cancer is prostate cancer and the method further comprising optionally obtaining an additional biological sample from the subject, measuring the level of alpha-actinin in the biological sample collected from the subject, determining that the level of alpha-actinin is higher than a predetermined value and selecting the subject for treatment for prostate cancer.

In another embodiment the invention provides a method for selecting a subject for treatment wherein the cancer is skin cancer and the method further comprising optionally obtaining an additional biological sample from the subject, measuring the level of alpha-actinin in the biological sample collected from the subject, determining that the level of alpha-actinin is higher than a predetermined value and selecting the subject for treatment for skin cancer.

In another embodiment the invention provides a method for selecting a subject for treatment wherein the cancer is breast cancer and the method further comprising optionally obtaining an additional biological sample from the subject, measuring the level of HER-2 in the biological sample collected from the subject, determining that the level of HER-2 is higher than a predetermined value and selecting the subject for treatment for breast cancer.

In another embodiment the invention provides a method for selecting a subject for treatment wherein the cancer is breast cancer and the method further comprising optionally obtaining an additional biological sample from the subject, measuring the level of estrogen receptor in the biological sample collected from the subject, determining that the level of estrogen receptor is higher than a predetermined value and selecting the subject for treatment for breast cancer.

In another embodiment the invention provides a method for selecting a subject for treatment wherein the cancer is hepatocellular carcinoma and the method further comprising optionally obtaining an additional biological sample from the subject, measuring the level of any of Hsp27, Hsp70, glucose-regulated protein 78 and Hcc-2 in the biological sample collected from the subject, determining that the level of any of Hsp27, Hsp70, glucose-regulated protein 78 and Hcc-2 is higher than a predetermined value and selecting the subject for treatment for hepatocellular carcinoma.

In another embodiment the invention provides a method for selecting a subject for treatment wherein the cancer is bladder cancer and the method further comprising optionally obtaining an additional biological sample from the subject, measuring the level of urinary nuclear matrix protein NMP22 in the biological sample collected from the subject, determining that the level of urinary nuclear matrix protein NMP22 is higher than a predetermined value and selecting the subject for treatment for bladder cancer.

In another embodiment the invention provides a method for selecting a subject for treatment wherein the cancer is ER(+)-breast cancer and the method further comprising optionally obtaining an additional biological sample from the subject, measuring the level of any of CRABP-II, cyclophilin A, neudesin and hemoglobin in the biological sample collected from the subject, determining that the level of any of CRABP-II, cyclophilin A, neudesin and hemoglobin is higher than a predetermined value and selecting the subject for tamoxifen treatment for ER(+)-breast cancer.

In another embodiment the invention provides a method for selecting a subject for treatment wherein the cancer is ER(+)-breast cancer and the method further comprising optionally obtaining an additional biological sample from the subject, measuring the level of any of cytochrome b5 and transgelin in the biological sample collected from the subject, determining that the level of any of cytochrome b5 and transgelin is lower than a predetermined value and selecting the subject for tamoxifen treatment for ER(+)-breast cancer.

In another embodiment the invention provides a method for selecting a subject for treatment wherein the cancer is B cell chronic lymphocytic leukemia and the method further comprising optionally obtaining an additional biological sample from the subject, measuring the level of Hsp27 in the biological sample collected from the subject, determining that the level of Hsp27 is higher than a predetermined value and selecting the subject for treatment for B cell chronic lymphocytic leukemia.

In another embodiment the invention provides a method for selecting a subject for treatment wherein the cancer is B cell chronic lymphocytic leukemia and the method further comprising optionally obtaining an additional biological sample from the subject, measuring the level of any of thioredoxin peroxidase 2 and protein disulfide isomerase in the biological sample collected from the subject, determining that the level of any of thioredoxin peroxidase 2 and protein disulfide isomerase is lower than a predetermined value and selecting the subject for treatment for B cell chronic lymphocytic leukemia.

In another embodiment the invention provides a method for selecting a subject for treatment wherein the cancer is prostate cancer and the method further comprising optionally obtaining an additional biological sample from the subject, measuring the level of prostate-specific antigen in the biological sample collected from the subject, determining that the level of prostate-specific antigen is higher than a predetermined value and selecting the subject for treatment for prostate cancer.

### DEFINITIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “administering” as used herein refers to oral administration, administration as a suppository, topical contact, intravenous, intraperitoneal, intramuscular, intralesional, intranasal or subcutaneous administration, or the implantation of a slow-release device, e.g., a mini-osmotic pump, to an individual. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

As described herein, “administered in combination,” or “a combination of” or the like, when referring to component (i), and component (ii), of what is presently described, is meant that the components are administered concurrently, or simultaneously to a mammal being treated. By concurrently or simultaneously, it is meant that each component may be administered at the same time or sequentially in any order at different points in time, however if not administered at the same time, they should be administered sufficiently closely in time so as to provide the desired treatment effect. Suitable dosing intervals and dosing order with such compounds will be readily apparent to those skilled in the art, once armed with the present disclosure. In one embodiment, all components are administered at the same time. In one embodiment, administration of a combination of 2-(4-ethoxyphenyl)-4-methyl-1-(4-sulfamoylphenyl)pyrrole and a second therapeutic agent or one or more therapeutic agents, as described herein, includes simultaneous (concurrent) and consecutive administration in any order.

As used herein, “cancer” refers to any of various malignant neoplasms characterized by the proliferation of anaplastic cells that tend to invade surrounding tissue and metastasize to new body sites. Examples of different types of cancer include, but are not limited to, lung cancer, breast cancer, bladder cancer, thyroid cancer, liver cancer, pleural cancer, pancreatic cancer, ovarian cancer, cervical cancer, testicular cancer, colon cancer, anal cancer, bile duct cancer, gastrointestinal carcinoid tumors, esophageal cancer, gall bladder cancer, rectal cancer, appendix cancer, small intestine cancer, stomach (gastric) cancer, renal cancer, cancer of the central nervous system, skin cancer, choriocarcinomas; head and neck cancers, blood cancers, osteogenic sarcomas, B-cell lymphoma, non-Hodgkin's lymphoma, Burkitt's lymphoma, fibrosarcoma, neuroblastoma, glioma, melanoma, monocytic leukemia, myelogenous leukemia, acute lymphocytic leukemia, and acute myelocytic leukemia.

A “COX enzyme” is used herein to refer to an enzyme having cyclooxygenase activity. These enzymes catalyze the formation of prostaglandins and thromboxane from arachidonic acid by means of their cyclooxygenase and peroxidase activities. Alternative names include: fatty acid cyclooxygenase, prostaglandin-endoperoxide synthase, prostaglandin-endoperoxide synthetase, prostaglandin synthase, prostaglandin synthetase, PG synthetase, (PG)H synthase, and prostaglandin G/H synthase. There are at least two isoforms of cyclooxygenase, referred to as COX-1 and COX-2. Alternative names for these enzymes include PGHS-1 and PGHS-2, respectively.

As used herein, “COX-2 selective inhibitor compound” or “COX-2 selective inhibitor” refers to a compound which inhibits COX-2 to a greater extent than it inhibits COX-1. Some non-limiting examples of COX-2 selective inhibitor compounds include: nimesulide, meloxicam, diclofenac, parecoxib (Dynastat®), celecoxib (Celebrex®), etoricoxib (Arcoxia®), lumiracoxib (Prexige), rofecoxib (Vioxx®), and valdecoxib (Bextra®). The disclosure should not be construed as being limited solely to these examples, as other COX-2 selective inhibitor compounds which are at present unknown, once known, may also be relevant in the methods described herein. In one embodiment, a COX-2 selective inhibitor compound has a selectivity ratio of cyclooxygenase-2 inhibition over cyclooxygenase-1 inhibition of at least about 5. In some embodiments, a COX-2 selective inhibitor compound has a selectivity ratio of cyclooxygenase-2 inhibition over cyclooxygenase-1 inhibition of at least about 10. In another embodiment, a COX-2 selective inhibitor compound has a selectivity ratio of cyclooxygenase-2 inhibition over cyclooxygenase-1 inhibition of at least about 50. In other embodiments, inhibition is assessed using a whole blood assay (FitzGerald and Patrono, 2001, N. Engl. J. Med. 345:433-442).

As described herein a 1,2-diphenylpyrrole derivative has the following formula:

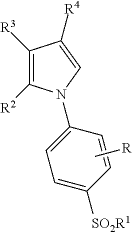

wherein:

R is a hydrogen atom, a halogen atom or an alkyl group having from 1 to 6 carbon atoms;

R¹ is an alkyl group having from 1 to 6 carbon atoms or an amino group;

R² is a phenyl group which is unsubstituted or is substituted by at least one substituent selected from the group consisting of substituents α and substituents β;

R³ is a hydrogen atom, a halogen atom or an alkyl group which has from 1 to 6 carbon atoms and which is unsubstituted or is substituted by at least one substituent selected from the group consisting of a hydroxy group, a halogen atom, an alkoxy group having from 1 to 6 carbon atoms and an alkylthio group having from 1 to 6 carbon atoms;

R⁴ is a hydrogen atom; an alkyl group which has from 1 to 6 carbon atoms and which is unsubstituted or is substituted by at least one substituent selected from the group consisting of a hydroxy group, a halogen atom, an alkoxy group having from 1 to 6 carbon atoms and an alkylthio group having from 1 to 6 carbon atoms; a cycloalkyl group having from 3 to 8 carbon atoms, an aryl group; or an aralkyl group; said aryl group having from 6 to 14 ring carbon atoms in a carbocyclic ring and are unsubstituted or are substituted by at least one substituent selected from the group consisting of substituents α and substituents β;

said aralkyl group are an alkyl group having from 1 to 6 carbon atoms and which are substituted by at least one aryl group as defined above;

said substituents α are selected from the group consisting of a hydroxy group, a halogen atom, an alkoxy group having from 1 to 6 carbon atoms and an alkylthio group having from 1 to 6 carbon atoms;

said substituents β are selected from the group consisting of an alkyl group which has from 1 to 6 carbon atoms and which is unsubstituted or are substituted by at least one substituent selected from the group consisting of a hydroxy group, a halogen atom, an alkoxy group having from 1 to 6 carbon atoms and an alkylthio group having from 1 to 6 carbon atoms; an alkanoyloxy group having from 1 to 6 carbon atoms; a mercapto group; an alkanoylthio group having from 1 to 6 carbon atoms; an alkylsulfinyl group having from 1 to 6 carbon atoms; a cycloalkloxy group having from 3 to 8 carbon atoms; a haloalkoxy group having from 1 to 6 carbon atoms; and an alkylenedioxy group having from 1 to 6 carbon atoms; or a pharmaceutically acceptable salt, solvate, or prodrug.

In one embodiment the derivative includes compounds wherein:

R is a hydrogen atom, a halogen atom or an alkyl group having from 1 to 4 carbon atoms;

R¹ is a methyl group or an amino group;

R² is an unsubstituted phenyl group or a phenyl group which is substituted by at least one substituent selected from the group consisting of a halogen atom; an alkoxy group having from 1 to 4 carbon atoms; an alkylthio group having from 1 to 4 carbon atoms; an unsubstituted alkyl group having from 1 to 4 carbon atoms; an alkyl group having from 1 to 4 carbon atoms and which is substituted by at least one substituent selected from the group consisting of a halogen atom, an alkoxy group having from 1 to 4 carbon atoms and an alkylthio group having from 1 to 4 carbon atoms; a haloalkoxy group having from 1 to 4 carbon atoms; and an alkylenedioxy group having from 1 to 4 carbon atoms;

R³ is a hydrogen atom, a halogen atom, an unsubstituted alkyl group having from 1 to 4 carbon atoms or a substituted alkyl group having from 1 to 4 carbon atoms and substituted by at least one substituent selected from the group consisting of a halogen atom, an alkoxy group having from 1 to 4 carbon atoms and an alkylthio group having from 1 to 4 carbon atoms;

R⁴ is a hydrogen atom; an unsubstituted alkyl group having from 1 to 4 carbon atoms; a substituted alkyl group having from 1 to 4 carbon atoms and substituted by at least one substituent selected from the group consisting of a hydroxy group, a halogen atom, an alkoxy group having from 1 to 4 carbon atoms and an alkylthio group having from 1 to carbon atoms; a cycloalkyl group having from 3 to 6 carbon atoms; an aryl group which has from 6 to 10 ring carbon atoms and which is unsubstituted or is substituted by at least one substituent selected from the group consisting of a halogen atom; an alkoxy group having from 1 to 4 carbon atoms; an alkylthio group having from 1 to 4 carbon atoms; an unsubstituted alkyl group having from 1 to 4 carbon atoms; an alkyl group having from 1 to 4 carbon atoms and substituted by at least one substituent selected from the group consisting of a hydroxy group, a halogen atom, an alkoxy group having from 1 to 4 carbon atoms and an alkylthio group having from 1 to 4 carbon atoms; and a cycloalkyloxy group having from 3 to 7 carbon atoms; an aralkyl group having from 1 to 4 carbon atoms in the alkyl part and containing at least one said aryl group; or a pharmaceutically acceptable salt, solvate, or prodrug.

In a further embodiment the derivative includes compounds wherein

R is a hydrogen atom;

R¹ is an amino group;

R² is an unsubstituted phenyl group or a phenyl group which is substituted by at least one substituent selected from the group consisting of a halogen atom, an alkoxy group having from 1 to 4 carbon atoms, an alkylthio group having from 1 to 4 carbon atoms, an alkyl group having from 1 to 4 carbon atoms, a haloalkyl group having from 1 to 4 carbon atoms, a haloalkoxy group having from 1 to 4 carbon atoms and a alkylenedioxy group having from 1 to 4 carbon atoms;

R³ is a hydrogen atom, a halogen atom, an alkyl group having from 1 to 4 carbon atoms or a haloalkyl group having from 1 to 4 carbon atoms;

R⁴ is a hydrogen atom; an unsubstituted alkyl group having from 1 to 4 carbon atoms; a substituted alkyl group having from 1 to 4 carbon atoms and substituted by at least one substituent selected from the group consisting of a hydroxy group and an alkoxy group having from 1 to 4 carbon atoms; a cycloalkyl group having from 3 to 6 carbon atoms; an aryl group which has from 6 to 10 ring carbon atoms and which is unsubstituted or is substituted by at least one substituent selected from the group consisting of a hydroxy group; a halogen atom; an alkoxy group having from 1 to 4 carbon atoms; an unsubstituted alkyl group having from 1 to 4 carbon atoms; an alkyl group having from 1 to 4 carbon atoms and which is unsubstituted or substituted by at least one halogen atom; and a cycloalkyloxy group having from 3 to 7 carbon atoms; and an aralkyl group having from 1 to 4 carbon atoms in the alkyl part and containing at least one said aryl group; or a pharmaceutically acceptable salt, solvate, or prodrug.

In still a further embodiment the derivative includes a compound selected from the group consisting of: 4-methyl-2-(4-methylphenyl)-1-(4-sulfamoylphenyl)pyrrole; 2-(4-methoxyphenyl)-4-methyl-1-(4-sulfamoylphenyl)pyrrole; 2-(4-chlorophenyl)-4-methyl-1-(4-sulfamoylphenyl)pyrrole; 4-methyl-2-(4-methylthiophenyl)-1-(4-sulfamoylphenyl)pyrrole; 2-(4-ethoxyphenyl)-4-methyl-1-(4-sulfamoylphenyl)pyrrole; 2-(4-methoxy-3-methylphenyl)-4-methyl-1-(4-sulfamoylphenyl)pyrrole; 2-(3-fluoro-4-methoxyphenyl)-4-methyl-1-(4-sulfamoylphenyl)pyrrole; 2-(3,4-dimethylphenyl)-4-methyl-1-(4-sulfamoylphenyl)pyrrole; 4-methyl-1-(4-methylthiophenyl)-2-(4-sulfamoylphenyl)pyrrole; 1-(4-acetylaminosulfonylphenyl)-4-methyl-2-(4-methoxyphenyl)pyrrole; and 1-(4-acetylaminosulfonylphenyl)-4-methyl-2-(3,4-dimethylphenyl)pyrrole.

Other COX-2 selective inhibitor compounds include but are not limited to 1,2-diphenylpyrrole derivatives which are described in U.S. RE 39,420, herein incorporated by reference, including their methods of preparation. By way of example only, a patient may be selected for treatment with a COX-2 selective inhibitor selected from the group consisting of: 4-methyl-2-(4-methylphenyl)-1-(4-sulfamoylphenyl)pyrrole; 2-(4-methoxyphenyl)-4-methyl-1-(4-sulfamoylphenyl)pyrrole; 2-(4-chlorophenyl)-4-methyl-1-(4-sulfamoylphenyl)pyrrole; 4-methyl-2-(4-methylthiophenyl)-1-(4-sulfamoylphenyl)pyrrole; 2-(4-ethoxyphenyl)-4-methyl-1-(4-sulfamoylphenyl)pyrrole; 2-(4-methoxy-3-methylphenyl)-4-methyl-1-(4-sulfamoylphenyl)pyrrole; 2-(3-fluoro-4-methoxyphenyl)-4-methyl-1-(4-sulfamoylphenyl)pyrrole; 2-(3,4-dimethylphenyl)-4-methyl-1-(4-sulfamoylphenyl)pyrrole; 4-methyl-1-(4-methylthiophenyl)-2-(4-sulfamoylphenyl)pyrrole; 1-(4-acetylaminosulfonylphenyl)-4-methyl-2-(4-methoxyphenyl)pyrrole; and 1-(4-acetylaminosulfonylphenyl)-4-methyl-2-(3,4-dimethylphenyl)pyrrole.

As used herein, “COX-2 selective inhibitor compound therapy” refers to any regular dosing of a COX-2 selective inhibitor compound.

As used herein, the term “inflammatory disease” refers to a disease or disorder characterized or caused by inflammation. “Inflammation” refers to a local response to cellular injury that is marked by capillary dilatation, leukocytic infiltration, redness, heat, and pain that serves as a mechanism initiating the elimination of noxious agents and of damaged tissue. The site of inflammation includes the lungs, the pleura, a tendon, a lymph node or gland, the uvula, the vagina, the brain, the spinal cord, nasal and pharyngeal mucous membranes, a muscle, the skin, bone or bony tissue, a joint, the urinary bladder, the retina, the cervix of the uterus, the canthus, the intestinal tract, the vertebrae, the rectum, the anus, a bursa, a follicle, and the like. Such inflammatory diseases include, but are not limited to, inflammatory bowel disease, rheumatoid diseases (e.g., rheumatoid arthritis), other arthritic diseases (e.g., acute arthritis, acute gouty arthritis, bacterial arthritis, chronic inflammatory arthritis, degenerative arthritis (osteoarthritis), infectious arthritis, juvenile arthritis, mycotic arthritis, neuropathic arthritis, polyarthritis, proliferative arthritis, psoriatic arthritis, venereal arthritis, viral arthritis), fibrositis, pelvic inflammatory disease, acne, psoriasis, actinomycosis, dysentery, biliary cirrhosis, Lyme disease, heat rash, Stevens-Johnson syndrome, mumps, pemphigus vulgaris, and blastomycosis. Inflammatory bowel diseases are chronic inflammatory diseases of the gastrointestinal tract which include, without limitation, Crohn's disease, ulcerative colitis, and indeterminate colitis. Rheumatoid arthritis is a chronic inflammatory disease primarily of the joints, usually polyarticular, marked by inflammatory changes in the synovial membranes and articular structures and by muscle atrophy and rarefaction of the bones.

As used herein, “inhibiting” refers to a detectable reduction in an activity or process resulting from administration of a drug compared to the activity or process prior to the administration of the drug.

As used herein, “inhibits COX-2” or “inhibiting COX-2” should be construed to include: inhibiting the enzymatic activity of COX-2, inhibiting the transcription of the COX-2 gene, and inhibiting the translation of COX-2 mRNA. Inhibiting the enzymatic activity of COX-2 includes reducing the half-life of COX-2 enzyme, for instance, by increasing degradation of either COX-2 and/or the mRNA for COX-2.

### NSAIDs

A “non-steroidal anti-inflammatory drug” (NSAID) is used herein to refer to a drug which has analgesic, antipyretic and anti-inflammatory effects. Traditional NSAIDs are non-selective inhibitors of both PGHS-1 and PGHS-2. Examples of non-selective NSAID inhibitors include: aspirin which irreversibly acetylates cyclooxygenase, propionic acid derivatives such as ibuprofen, naproxen, acetic acid derivatives such as indomethacin, and meclofenamic acid, and several other classes of organic acids such as piroxicam, all of which compete with arachidonic acid at the active site of cyclooxygenase.

The inhibition of COX-2 is thought to mediate, at least in part, the antipyretic, analgesic, and anti-inflammatory actions of NSAIDs, but the simultaneous inhibition of COX-1 results in unwanted side effects, particularly those leading to gastric ulcers that result from decreased prostaglandin formation. Acetaminophen is a very weak anti-inflammatory drug, but is effective as an antipyretic and analgesic agent, and lacks certain side effects of NSAIDs, such as gastrointestinal tract damage and blockade of platelet aggregation. Patients who use NSAIDs on a chronic basis have about three times greater relative risk for serious adverse gastrointestinal events compared to nonusers. NSAIDs vary considerably in their tendency to cause such erosions and ulcers. Gastric damage by these agents can be brought about by at least two distinct mechanisms. Although local irritation by orally administered drugs allows back diffusion of acid into the gastric mucosa and induces tissue damage, parenteral administration also can cause damage and bleeding, correlated with inhibition of the biosynthesis of prostaglandins, especially PGI₂ and PGE₂ that serve as cytoprotective agents in the gastric mucosa. These eicosanoids inhibit acid secretion by the stomach, enhance mucosal blood flow, and promote the secretion of cytoprotective mucus in the intestine; inhibition of their synthesis may render the stomach more susceptible to damage. All of the NSAIDs, with the exception of p-aminophenol derivatives, have a tendency to cause gastrointestinal side effects, ranging from mild dyspepsia and heartburn to ulceration of the stomach or duodenum, sometimes with fatal results.

In describing the present disclosure, the terms “patient,” “person,” and “individual” refers to a human. The human can be any human.

As described herein, a “pre-determined PGE-M level” may be a level of PGE-M used to, by way of example only, evaluate a patient that may be selected for treatment, evaluate a response to a COX-2 inhibitor treatment, evaluate a response to a combination of a COX-2 inhibitor and a second therapeutic agent treatment, and/or diagnose a patient for cancer, inflammation, pain and/or related conditions. A pre-determined PGE-M level may be determined in populations of patients with or without cancer. The pre-determined PGE-M level can be a single number, equally applicable to every patient, or the pre-determined PGE-M level can vary according to specific subpopulations of patients. For example, men might have a different pre-determined PGE-M level than women; non-smokers may have a different pre-determined PGE-M level than smokers. Age, weight, and height of a patient may affect the pre-determined PGE-M of the individual. Furthermore, the pre-determined PGE-M level can be a level determined for each patient individually. The pre-determined PGE-M level can be any suitable standard. For example, the pre-determined PGE-M level can be obtained from the same or a different human for whom a patient selection is being assessed. In one embodiment, the pre-determined PGE-M can be obtained from a previous assessment of the same patient. In such a manner, the progress of the selection of the patient can be monitored over time. In addition, the standard can be obtained from an assessment of another human or multiple humans, e.g., selected groups of humans. In such a manner, the extent of the selection of the human for whom selection is being assessed can be compared to suitable other humans, e.g., other humans who are in a similar situation to the human of interest, such as those suffering from similar or the same condition(s). Moreover, the standard may reflect normal and/or abnormal levels of the urinary metabolite of PGE₂ from a general population of humans.

As used herein, the term “sample” refers to any biological specimen, fluid, cell, tissue, organ or portion thereof, that includes a cancerous cell, such as a cell from the colon, rectum, breast, ovary, prostate, kidney, lung, blood, brain or other organ or tissue that contains or is suspected to contain a cancerous cell. By way of example only, a sample may include a solid tissue, plasma, cerebrospinal fluid, pleural fluid, peritoneal fluid, amniotic fluid, saliva, sputum, mucus, bone marrow, serum, whole blood, urine, lymph, stool, feces, semen, tears, and any other bodily fluid, and cellular extracts. The term includes samples present in an individual as well as samples obtained or derived from the individual. For example, a sample can be a histologic section of a specimen obtained by biopsy, or cells that are placed in or adapted to tissue culture. A sample further can be a subcellular fraction or extract, or a crude or substantially pure nucleic acid molecule or protein preparation.

As described herein, “therapeutically effective amount” may be an amount of component (i), and component (ii) that when administered alone or in combination to a patient is effective to treat the condition. By way of example only, a “therapeutically effective amount” is a nontoxic but sufficient amount of an agent to provide the desired biological result. That result can be reduction and/or alleviation of the signs, symptoms, or causes of a disorder, or any other desired alteration of a biological system. An appropriate therapeutic amount in any individual case may be determined by one of ordinary skill in the art using routine experimentation. Methods presently disclosed herein, give the opportunity of obtaining reductions in PGE-M levels of a patient, thereby diminishing the side effects and possible toxicity which would result from the otherwise required amounts of the individual drug components.

As used herein, “treating inflammation and/or pain” means reducing the frequency with which inflammation and/or pain is experienced by a patient. “Treating inflammation and/or pain” also encompasses alleviating inflammation and/or pain, which means the severity of the symptom is reduced.

### PGE₂

“PGE₂,” as used herein, refers to prostaglandin E₂ and is known as one of the metabolites in an arachidonate cascade. PGE₂ is also known to have various activities such as pain inducing activity, inflammatory activity, uterine contractile activity, a promoting effect on digestive peristalsis, an awaking activity, a suppressive effect on gastric acid secretion, hypotensive activity, blood platelet inhibition activity, bone-resorbing activity, angiogenic activity, or the like.

Several observations suggest that PGE₂ contributes to the development and progression of cancer. For example, PGE₂ can stimulate cell proliferation, induce angiogenesis, inhibit apoptosis, and suppress immune surveillance (Ben-Av, “Induction of vascular endothelial growth factor expression in synovial fibroblasts by prostaglandin E and interleukin-1: A potential mechanism for inflammatory angiogenesis,” F.E.B.S. Lett., 372: 83-87 (1995); Dannenberg, “Targeting cyclooxygenase-2 in human neoplasia: rationale and promise,” Cancer Cell, 4: 431-36 (2003); Masferrer, “Antiangiogenic and antitumor activities of cyclooxygenase-2 inhibitors,” Cancer Res., 60: 1306-11 (2000); Sheng, “Modulation of apoptosis and Bcl-2 expression by prostaglandin E2 in human colon cancer cells,” Cancer Res., 58: 362-66 (1998); Stolina, “Specific inhibition of cyclooxygenase-2 restores antitumor reactivity by altering the balance of IL-10 and IL-12 synthesis,” J. Immunol., 164: 361-70 (2000)). Treatment with selective inhibitors of COX-2, prototypic inhibitors of PGE₂ synthesis, or an anti-PGE₂ monoclonal antibody has been shown to inhibit tumor growth of transplantable tumors of the UADT including HNSCC (Zweifel, “Direct evidence for a role of cyclooxygenase-2 derived prostaglandin E2 in human head and neck xenograft tumors,” Cancer Res., 62: 6706-11 (2002)). Further, exposure to tobacco smoke was found to stimulate COX-2 transcription resulting in enhanced PGE₂ synthesis in cells derived from the UADT (Martey, “Cigarette smoke induces cyclooxygenase-2 and microsomal prostaglandin E2 synthase in human lung fibroblasts: implications for lung inflammation and cancer,” Am. J. Physiol. Lung Cell Mol. Physiol., 287: L981-L991 (2004); Moraitis, “Levels of cyclooxygenase-2 are increased in the oral mucosa of smokers: evidence for the role of epidermal growth factor receptor and its ligands,” Cancer Res., 65: 664-70 (2005)).

Accordingly, PGE₂ is appropriate for use as a biomarker of the carcinogenic effects of tobacco smoke as well as other conditions associated with lung injury. Tissue measurements of PGE₂ are generally invasive for routine clinical use. Moreover, PGE₂ in plasma is rapidly metabolized in the lungs and, therefore, does not always accurately reflect endogenous PGE₂ production (Piper, “Inactivation of prostaglandins by the lungs,” Nature, 225: 600-04 (1970)).

### PGE₂ Metabolite

As used herein, “PGE₂ metabolite,” refers to a byproduct of PGE₂ metabolism in an animal, such as by way of example only, a mammal. “PGE-M” refers to the PGE₂ metabolite 9,15-dioxo-11α-hydroxy-2,3,4,5-tetranor-prostan-1,20-dioic-17,17,18,18,19,19-d₆ acid. PGE-M can be detected and quantified from samples including, but not limited to, urine, plasma, serum, cerebrospinal fluid, saliva, semen, pleural fluid, peritoneal fluid and amniotic fluid.

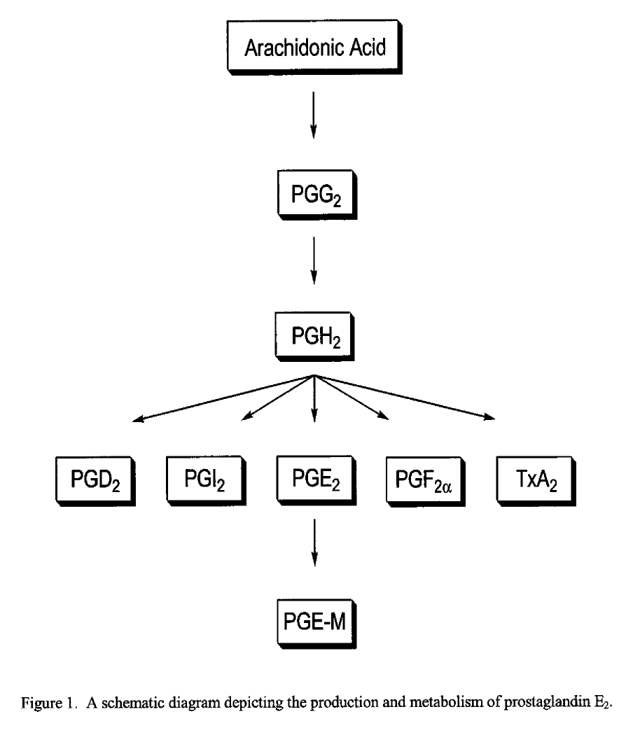

The schematic diagram of FIG. 1 illustrates that enhanced production of PGE-M can potentially result from increased levels of phospholipase A2, COX-2, COX-1, or mPGES-1, each of which may lead to elevated levels of PGE₂ and its subsequent metabolism to PGE-M (Sheng, “Prostaglandin E2 increases growth and motility of colorectal carcinoma cells,” J. Biol. Chem., 276: 18075-81 (2001)). The source of the PGE₂, and hence PGE-M, may vary. The lung is the most likely source in smokers, due to its immense surface area and the known link between pulmonary inflammation and agents such as tobacco smoke. Further, inflammation is associated with increased production of PGE₂, and various cell types within the lung have the capacity to produce large quantities of PGE₂ in response to pro-inflammatory stimuli (Mao, “Modulation of pulmonary leukotriene B4 production by cyclooxygenase-2 inhibitors and lipopolysaccharide,” Clin. Cancer Res., 10: 6872-78 (2004); Mao, “Celecoxib modulates the capacity for prostaglandin E2 and interleukin-10 production in alveolar macrophages from active smokers,” Clin. Cancer Res., 9: 5835-41 (2003)). Previous studies have demonstrated that urinary PGE-M can be used as an index of systemic PGE₂ production (Ferretti, “Quantitative analysis of 11-alpha-hydroxy-9,15-dioxo-2,3,4,5,20-pentanor-19-carboxyprostanoic acid, the major urinary metabolite of E prostaglandins in man,” Anal. Biochem., 128: 351-58 (1983); Seyberth, “Quantifications of the major urinary metabolite of E prostaglandins by mass spectrometry: evaluation of the method's application to clinical studies,” Prostaglandins, 11: 381-97 (1976)).

### Urinary PGE-M

Urinary PGE-M is a biomarker of changes in tumor-derived COX-2 activity. Patients being treated with COX-2 inhibitors who exhibit the greatest proportional decline in urinary PGE-M levels experience a longer survival compared to those with no change or an increase in PGE-M. In one embodiment, is a method for selecting a patient for treatment when a measured PGE-M level is above that of a pre-determined PGE-M level. In one embodiment, the method provides PGE-M as a marker whose expression patterns correlate with important characteristics of COX-2 inhibitor responsiveness of certain patient populations. Methods are provided for the use of PGE-M as a marker to distinguish between cancer patient groups, to determine general courses of treatment, and to ascertain patient prognosis. Additionally, methods for use of PGE-M as a marker to distinguish between patients who will respond to COX-2 inhibitor therapy for the treatment of inflammation and related conditions, pain, and/or conditions associated with cancer are similarly provided. As will be described in greater detail below, assays for measuring PGE-M levels are likewise provided.

In one aspect, is a method of selecting a patient for treatment with 2-(4-ethoxyphenyl)-4-methyl-1-(4-sulfamoylphenyl)pyrrole wherein a PGE-M level from a patient's urine sample is above that of a pre-determined PGE-M level. In one embodiment, is a method of selecting a patient for treatment with 2-(4-ethoxyphenyl)-4-methyl-1-(4-sulfamoylphenyl)pyrrole comprising determining a PGE-M level of a patient sample and selecting the patient for treatment with 2-(4-ethoxyphenyl)-4-methyl-1-(4-sulfamoylphenyl)pyrrole provided that the PGE-M level is above a pre-determined PGE-M level. In another embodiment is a method of selecting a patient for treatment with 244-ethoxyphenyl)-4-methyl-1-(4-sulfamoylphenyl)pyrrole wherein determining a PGE-M level of a patient sample comprises collecting the sample from the patient and measuring the PGE-M level of the patient sample. In a further embodiment is a method of selecting a patient for treatment with 2-(4-ethoxyphenyl)-4-methyl-1-(4-sulfamoylphenyl)pyrrole wherein the patient sample is selected from the group consisting of a solid tissue, saliva, sputum, mucus, bone marrow, serum, blood, urine, lymph, tears, semen, or stool.

In another embodiment the sample is urine. In yet another embodiment, the sample is a solid tissue. In a further embodiment, the solid tissue is a tumor. In yet a further embodiment, the tumor is selected from the group consisting of: squamous cell carcinoma, basal cell carcinoma, transitional cell carcinoma, adenocarcinoma, malignant gastrinoma, cholangeiocellular carcinoma, hepatocellular carcinoma, renal cell carcinoma, malignant melanoma, fibrosarcoma, myxosarcoma, liposarcoma, leiomyosarcoma, rhabdomyosarcoma, malignant teratoma, hemangiosarcoma, Kaposi sarcoma, lymphangiosarcoma, osteosarcoma, chondrosarcoma, malignant meningioma, non-Hodgkin lymphoma, Hodgkin lymphoma, and leukemia. In another embodiment, the PGE-M level of a patient's urine sample is determined via mass spectroscopy. In a further embodiment, the PGE-M level of a patient's urine sample is determined via liquid chromatography/tandem mass spectroscopy.

In one embodiment, the present disclosure provides a useful marker for classifying patients into different prognostic categories. For example, one embodiment provides a method for using PGE-M as a marker to determine whether an individual afflicted with a particular type of cancer will have a good or poor clinical prognosis. The disclosure further provides recommended therapeutic regimens based upon whether PGE-M levels decrease following treatment with a COX-2 inhibitor. In one embodiment, PGE-M is utilized as a marker to initially identify patients exhibiting a higher than normal PGE-M levels prior to treatment with a COX-2 inhibitor. Patients demonstrating elevated levels of PGE-M in their urine will be selected for COX-2 inhibitor therapy. In one embodiment, of the patients demonstrating elevated levels of PGE-M in their urine prior to COX-2 inhibitor therapy, a sub-population of patients are identified based upon a demonstrated decrease of PGE-M after a pre-determined period of time; between about one and two weeks, between about eight and twelve days, and between about ten days. The decline in PGE-M levels over time is correlated to improved survival, improved disease prognosis, tumor growth inhibition, a heightened response to COX-2 inhibition therapy, and/or a heightened response to a combination of COX-2 inhibitor and a second therapeutic agent treatment.

The use of PGE-M as a marker is not restricted to the prognosis of certain cancer-related conditions, and may be applied in a variety of conditions, clinical or experimental, in which prostaglandin expression, or over-expression, plays a role. For example, elevated PGE-M levels in the urine may be the diagnosis and/or prognosis of clinical states or phenotypes associated with cancer-related disorders such as pain and cachexia, as well as other disease conditions in which prostaglandin expression is involved. These physiological conditions include inflammation, pyrexia, and pain, wherein PGE-M expression level data correlates with the particular physiological or disease condition and reduction of PGE-M expression similarly correlates with a patient's responsiveness to COX-2 inhibitor therapy.

In one aspect is a method of treating a patient with a cancer-related disorder such as cachexia wherein the PGE-M level of a patient sample is greater than a pre-determined PGE-M level comprising administering to the patient a therapeutically effective amount of 2-(4-ethoxyphenyl)-4-methyl-1-(4-sulfamoylphenyl)pyrrole. In one embodiment is a method of treating a patient with a cancer-related disorder such as cachexia wherein the PGE-M level of a patient sample is greater than a pre-determined PGE-M level comprising administering to the patient a combination of 2-(4-ethoxyphenyl)-4-methyl-1-(4-sulfamoylphenyl)pyrrole and a second therapeutic agent. In another embodiment is a method of selecting a patient for treatment of cachexia with 2-(4-ethoxyphenyl)-4-methyl-1-(4-sulfamoylphenyl)pyrrole comprising determining a PGE-M level and selecting the patient for treatment wherein the PGE-M level of the patient is greater than a pre-determined PGE-M level. In a further embodiment, is a method of selecting a patient for treatment of cachexia with a combination of 2-(4-ethoxyphenyl)-4-methyl-1-(4-sulfamoylphenyl)pyrrole and a second therapeutic agent comprising determining a PGE-M level and selecting the patient for treatment wherein the PGE-M level of the patient is greater than a pre-determined PGE-M level.

In another aspect is a method of treating a patient with a cancer-related disorder such as pain wherein the PGE-M level of a patient sample is greater than a pre-determined PGE-M level comprising administering to the patient a therapeutically effective amount of 2-(4-ethoxyphenyl)-4-methyl-1-(4-sulfamoylphenyl)pyrrole. In one embodiment is a method of treating a patient with a cancer-related disorder such as pain wherein the PGE-M level of a patient sample is greater than a pre-determined PGE-M level comprising administering to the patient a combination of 2-(4-ethoxyphenyl)-4-methyl-1-(4-sulfamoylphenyl)pyrrole and a second therapeutic agent. In another embodiment is a method of selecting a patient for treatment of pain with 2-(4-ethoxyphenyl)-4-methyl-1-(4-sulfamoylphenyl)pyrrole comprising determining a PGE-M level and selecting the patient for treatment wherein the PGE-M level of the patient is greater than a pre-determined PGE-M level. In a further embodiment, is a method of selecting a patient for treatment of pain with a combination of 2-(4-ethoxyphenyl)-4-methyl-1-(4-sulfamoylphenyl)pyrrole and a second therapeutic agent comprising determining a PGE-M level and selecting the patient for treatment wherein the PGE-M level of the patient is greater than a pre-determined PGE-M level.

The level of the urinary metabolite of PGE₂, e.g., PGE-M, in the human can be determined in any suitable manner. In one embodiment, the level of the urinary metabolite is determined by obtaining a urine sample from the human and subjecting the urine sample to suitable analysis, e.g., mass spectroscopy.

The PGE₂ or the PGE₂ metabolite isolated may be measured using an assay method for a prostaglandin. By way of example only, the assay is a quantitative assay. The level of PGE₂ or the PGE₂ metabolite is quantified based on the assay results using, for example, peak area or peak height ratios. An example of a preferred quantitative assay for a PGE₂ metabolite is described herein in the Examples.

By way of example only, the PGE₂ or the PGE₂ metabolite isolated as described above can be measured as follows. Briefly, after potassium hydroxide hydrolysis, the sample which contains PGE₂ or the PGE₂ metabolite is spiked with a known amount of a synthetic homologous internal standard. A non-limiting example of an internal standard includes a radio-labeled synthetic homologous PGE₂ metabolite. The samples are then subjected to solid phase extraction, derivatized, and purified using thin layer chromatography. After thin layer chromatography, each sample is analyzed for PGE₂ or the PGE₂ metabolite content using gas chromatography-mass spectrometry, and quantitation is performed using peak area or peak height ratios of the radio-labeled synthetic homologous internal standard molecule and the PGE₂ or the PGE₂ metabolite molecule of interest.

Measurements made using a tissue sample can be made using any tissue sample obtained from any type of tissue. Measurements made using a sample of body fluid can be made in any type of body fluid. By way of example only, the body fluid sample is a sample obtained from the group consisting of plasma and urine. In one embodiment, the body fluid sample is urine.

Urine is collected in sterile containers, typically 30 minutes after voiding. Alternatively, urine is collected as a time integrated sample. For instance, after voiding, urine is collected for a time period, for instance 2, 4 or 6 hours. If the urine sample is not analyzed immediately, the sample is stored in such a way as to prevent or reduce breakdown of the component(s) to be measured in the urine. One method of storage to prevent or reduce breakdown is to freeze the sample on dry ice immediately after collection and store the frozen sample at −70.degree. C. Marker levels in urine are generally normalized to another urine component. Typically, the other urine component is creatinine. Creatinine is measured using an automated colorimetric assay (Sigma-Aldrich Co., St Louis, Mo.).

Urinary PGE-M level is typically measured using a liquid chromatography/tandem mass spectrometric method as described in Murphey, L. J. et al.: “Quantification of major urinary metabolite of PGE₂ by a liquid chromatographic/mass spectrometric assay: Determination of cyclooxygenase specific PGE₂ synthesis in healthy humans and those with lung cancer” Anal Biochem 334: 266-75 (2004), the entire contents of which are hereby incorporated by reference.

Briefly, in one embodiment, 0.75 mL urine is acidified to pH 3 with HCl and endogenous PGE-M is then converted to 0-methyloxime derivative by treatment with methyloxime HCl. The methoximated PGE-M is extracted, applied to a C-18 Sep-Pak, and eluted with ethyl acetate. An [²H₆]-O-methyloxime PGE-M internal standard is then added. Liquid chromatography is performed on a Zorbax Eclipse XDB-C18 column attached to a Thermo Finnigan Surveyor MS Pump (Thermo Finnigan, San Jose, Calif.). For endogenous PFE-M, the predominant product ion m/z 336 representing [M-(OCH₃+H₂O]⁻ and the analogous ion, m/z 339 (M-OC[²H₃+H₂O), for the deuterated internal standard, are monitored in the selected reaction monitoring (SRM) mode. Quantification of endogenous PFE-M utilizes the ratio of the mass chromatogram peak areas of the m/z 336 and m/z 339 ions. The lower limit of detection of PGE-M is in the range of 40 pg, approximately 100-fold below levels in normal human urine. The coefficient in variation for samples analyzed in multiple batches is approximately 7.2%. Urinary creatinine levels are measured using a test kit from SIGMA Company (St. Louis, Mo.). Urine samples for each case-control pair are analyzed in the same batch and adjacently to eliminate between-assay variability. Individuals having elevated PGE-M levels relative to control urine are identified and administered COX-2 inhibitor therapy as will be described in greater detail below.

### Other Prostaglandin Biomarkers

PGI₂

“PGI₂” as used herein refers to prostacyclin, also known as epoprostenol and limits the cardiovascular effects of thromboxane A₂ (TxA₂), the major PGHS-1 product of platelets (Cheng et al., 2002, Science 296: 539-541). The cardiovascular effects of TxA₂ include: platelet aggregation (Thomas et al., 1998, J. Clin. Invest. 102:1994-2001), elevation of blood pressure (Qi et al., 2002, J. Clin. Invest. 110: 61-9; Francois et al., 2004, Hypertension 43:364-9) and acceleration of atherogenesis (Kobayashi et al., 2004, J. Clin. Invest. 114:784-94; Cayatte et al., 2000, Arterioscler. Thromb. Vasc. Biol. 20: 1724-8; Huo et al., 2003, Nat. Med. 9: 61-7).

All of the coxibs depress substantially the level of prostacyclin (PGI₂), leaving platelet COX-1-derived thromboxane A₂(TxA₂) level unaffected (McAdam et al., 1999, Proc. Natl. Acad. Sci. USA 96: 272-7; Catella-Lawson et al., 1999, J. Pharmacol. Exp. Ther. 289; 735-41). PGI₂, the dominant product of arachidonic acid in macrovascular endothelial cells, is formed by prostacyclin synthase (POTS) action on prostaglandin endoperoxide intermediates, which are produced catalytically by PGHS-2 (Moncada et al., 1976, Nature 263: 663-5). PGI₂ exhibits properties of potential relevance to atheroprotection. Specifically, it inhibits platelet aggregation, vascular smooth muscle contraction and proliferation (Cheng et al., 2002, Science 296: 539-541), leukocyte-endothelial cell interactions (Della Bella et al., 2001, Prostaglandins 65: 73-83) and cholesteryl ester hydrolase (Gryglewski et at, 1995, Ann. N.Y. Acad. Sci. 748: 194-206; discussion 206-7). It also activates reverse cholesterol transport (Morishita et al., 1990, J. Clin. Invest. 86: 1885-91). Indirect evidence suggests that PGI₂ protects against oxidant-induced tissue injury. Deletion of the PGI₂ receptor (IP) or suppression of PGI₂ biosynthesis augments cardiac injury caused by ischemia/reperfusion (Xiao et al., 2001, Circulation 104: 2210-5) or the anthracycline, doxarubacin (Dowd et al., 2001, J. Clin. Invest. 108: 585-90).

### Urinary PGI₂

“PGI₂ metabolite” as used herein refers to a byproduct of prostacyclin metabolism in an animal, such as a mammal. Non-limiting examples of PGI₂ metabolites are 2,3-dinor-6-keto PGF_{1α} and 15-keto-13,14-dihydro-2,3-dinor-6-keto-PGF_{1α}. The most abundant PGI₂ metabolite in human urine is 2,3-dinor-6-keto PGF_{1α}. As used herein, “PGIM” refers to the prostacyclin metabolite 2,3-dinor-6-keto-PGF_{1α}.

In one aspect, the invention provides a method for selecting a patient for treatment with 2-(4-ethoxyphenyl)-4-methyl-1-(4-sulfamoylphenyl)pyrrole comprising determining the PGI-M level in a patient sample wherein the PGI-M level is indicative of a need for treatment. In one embodiment, is a method for selecting a patient for treatment with a combination of 2-(4-ethoxyphenyl)-4-methyl-1-(4-sulfamoylphenyl)pyrrole and a second therapeutic agent comprising determining the PGI-M level in a patient sample wherein the PGI-M level is indicative of a need for treatment. In another embodiment, is a method for treating a patient wherein the PGI-M level of a patient sample is indicative of a need for treatment comprising administering to the patient a therapeutically effective amount of 2-(4-ethoxyphenyl)-4-methyl-1-(4-sulfamoylphenyl)pyrrole. In another embodiment is a method for treating a patient wherein the PGI-M level of a patient sample is indicative of a need for treatment comprising administering to the patient a combination of 2-(4-ethoxyphenyl)-4-methyl-1-(4-sulfamoylphenyl)pyrrole and a second therapeutic agent.

In one aspect is a method of assessing a therapy with 2-(4-ethoxyphenyl)-4-methyl-1-(4-sulfamoylphenyl)pyrrole, the method comprising the steps of measuring the level of a PGI₂ metabolite in the urine of the patient prior to administration of 2-(4-ethoxyphenyl)-4-methyl-1-(4-sulfamoylphenyl)pyrrole, measuring the level of PGI₂ metabolite in the urine of the subject at about four hours after administration of 2-(4-ethoxyphenyl)-4-methyl-1-(4-sulfamoylphenyl)pyrrole, and determining a ratio by dividing the level of PGI₂ metabolite in the urine of the subject at about four hours after administration of the compound by the level of PGI₂ metabolite in the urine of the subject prior to administration of the compound, wherein the subject has not been administered a non-steroidal anti-inflammatory drug or a COX-2 selective inhibitor compound for at least two weeks prior to administration of 2-(4-ethoxyphenyl)-4-methyl-1-(4-sulfamoylphenyl)pyrrole, and wherein a ratio of the level of PGI₂ metabolite at about four hours after 2-(4-ethoxyphenyl)-4-methyl-1-(4-sulfamoylphenyl)pyrrole administration to the level of PGI₂ metabolite prior to 2-(4-ethoxyphenyl)-4-methyl-1-(4-sulfamoylphenyl)pyrrole administration in the lower quartile is indicative of a need for treatment.

By way of example only, a method of measuring the level of urinary 2,3-dinor-6-keto PGF_{1α} is described herein in the Examples. In brief, urinary 2,3-dinor-6-keto PGF_{1α} is measured by a stable isotope dilution method, using gas chromatography/mass spectrometry (GC/MS). Briefly, the isoprostane biomarker is isolated by first, in the case of a tissue sample, homogenizing the tissue sample. In the case of a body fluid sample, no homogenization step is necessary. Total lipids are then extracted from the sample using ice-cold Folch solution, chloroform/methanol (2:1, v/v). The solution is then centrifuged briefly, and the organic phase, which contains the extracted lipids, is dried under nitrogen. Lipids are then hydrolyzed using aqueous potassium hydroxide to release the prostacyclin metabolite. Briefly, after potassium hydroxide hydrolysis, the sample which contains an prostacyclin metabolite is spiked with a known amount of a synthetic homologous internal standard. A non-limiting example of an internal standard includes a radio-labeled synthetic homologous prostacyclin metabolite. The samples are then subjected to solid phase extraction, derivatized, and purified using thin layer chromatography. After thin layer chromatography, each sample is analyzed for prostacyclin metabolite using gas chromatography-mass spectrometry, and quantitation is performed using peak area or peak height ratios of the radio-labeled synthetic homologous prostacyclin metabolite and the prostacyclin metabolite of interest.

### Prostanoid Metabolites

In other embodiments, the methods and compositions presently disclosed employ metabolites of a class of molecules called prostanoids. Prostanoids are derived from arachidonic acid, which is a fatty acid derivative, by the action of COX enzymes, and include prostaglandins, prostacyclin and thromboxanes. Specifically, the methods and compositions described herein assess levels of one or more prostanoid metabolites in biological sample of a mammal as a means of identifying mammal at risk of cancer, inflammation, pain, and/or related disorders. By way of example only, the biological sample is urine and the mammal is human.

The prostanoid metabolite can be isolated from the biological sample and the level measured using any method known to the skilled artisan for isolating a prostaglandin molecule and are described herein by way of example. Techniques and methods for measuring a prostanoid metabolite are known to the skilled artisan. Such methods include, but are not limited to, solvent extractions, solid phase extractions, centrifugation and sedimentation methods, quantitative and semi-quantitative methods such as chromatographic methods including thin layer chromatography, low, medium, and high pressure liquid chromatography methods, mass spectrometry methods, gas chromatography methods, gas chromatography/mass spectrometry methods, and immunological methods.

### EXAMPLES

The present disclosure is now described with reference to the following Examples. These Examples are provided for the purpose of illustration only and the present disclosure should in now way be construed as being limited to these Examples, but rather should be construed to encompass any and all variations which become evident as a result of the disclosure provided herein.

### Example 1

### Treatment with 2-(4-ethoxyphenyl)-4-methyl-1-(4-sulfamoylphenyl)pyrrole Based on patient PGEM level

This example illustrates a method of treating a subject with the COX-2 selective inhibitor compound 2-(4-ethoxyphenyl)-4-methyl-1-(4-sulfamoylphenyl)pyrrole comprising: the method comprising: measuring the level of PGEM in a biological sample collected from the subject prior to administration of the compound, determining that the PGEM level in the sample is higher than a predetermined value and administering to the subject the COX-2 selective inhibitor.

### Example 2

### Treatment with a combination comprising 2-(4-ethoxyphenyl)-4-methyl-1-(4-sulfamoylphenyl)pyrrole and a second agent based on PGEM level

This example illustrates a method of treating a subject with a combination comprising the COX-2 selective inhibitor compound 2-(4-ethoxyphenyl)-4-methyl-1-(4-sulfamoylphenyl)pyrrole and a second agent or therapy, the method comprising: measuring the level of PGEM in a biological sample collected from the subject prior to administration of the compound, determining that the PGEM level in the sample is higher than a predetermined value and administering to the subject the COX-2 selective inhibitor.

### Example 3

### Treatment with a combination comprising 2-(4-ethoxyphenyl)-4-methyl-1-(4-sulfamoylphenyl)pyrrole and a second agent based on PGEM ratio

This example illustrates a method of treating a subject with a combination comprising the COX-2 selective inhibitor compound 2-(4-ethoxyphenyl)-4-methyl-1-(4-sulfamoylphenyl)pyrrole and a second agent or therapy the method comprising: measuring the level of PGEM in a first biological sample collected from the subject prior to administration of the compound, measuring the level of PGEM in a second biological sample collected from the subject after administration of the second agent or therapy, and determining a ratio by dividing the level of PGEM in the second sample by the level of PGEM in the first sample, determining that the ratio is higher than a predetermined value and administering to the subject the combination of the COX-2 selective inhibitor compound 2-(4-ethoxyphenyl)-4-methyl-1-(4-sulfamoylphenyl)pyrrole and the second agent or therapy.

### Example 4

### Treatment with 2-(4-ethoxyphenyl)-4-methyl-1-(4-sulfamoylphenyl)pyrrole PGE2 metabolite level

This example illustrates a method of treating a subject with the COX-2 selective inhibitor compound 2-(4-ethoxyphenyl)-4-methyl-1-(4-sulfamoylphenyl)pyrrole comprising: measuring the level of PGE2 metabolite in a biological sample collected from the subject prior to administration of the compound, determining that the PGE2 metabolite level in the sample is higher than predetermined value and administering to the subject the COX-2 selective inhibitor.

### Example 5

### Treatment with a combination of 2-(4-ethoxyphenyl)-4-methyl-1-(4-sulfamoylphenyl)pyrrole and a second agent or therapy based on PGE2 metabolite level

This example illustrates a method of treating a subject with a combination comprising the COX-2 selective inhibitor compound 2-(4-ethoxyphenyl)-4-methyl-1-(4-sulfamoylphenyl)pyrrole and a second agent or therapy, the method comprising: measuring the level of PGE2 metabolite in a biological sample collected from the subject prior to administration of the compound, determining that the PGE2 metabolite level in the sample is higher than a predetermined value and administering to the subject the COX-2 selective inhibitor.

### Example 6

### Treating with a combination comprising 2-(4-ethoxyphenyl-1)-4-methyl-1-(4-sulfamoylphenyl)pyrrole and a second agent or therapy based on PGE2 metabolite ratio

This example illustrates a method of treating a subject with a combination comprising the COX-2 selective inhibitor compound 2-(4-ethoxyphenyl)-4-methyl-1-(4-sulfamoylphenyl)pyrrole and a second agent or therapy the method comprising: measuring the level of PGE2 metabolite in a first biological sample collected from the subject prior to administration of the compound, measuring the level of PGE2 metabolite in a second biological sample collected from the subject after administration of the second agent or therapy, and determining a ratio by dividing the level of PGE2 metabolite in the second sample by the level of PGE2 metabolite in the first sample, determining that the ratio is higher than a predetermined value and administering to the subject the combination of the COX-2 selective inhibitor compound 2-(4-ethoxyphenyl)-4-methyl-1-(4-sulfamoylphenyl)pyrrole and the second agent or therapy.

### Example 7

### Treatment with 2-(4-ethoxyphenyl)-4-methyl-1-(4-sulfamoylphenyl)pyrrole based on PGEM level higher than 7 ng/mg for a female subject or higher than about 12 ng/mg for a male subject

This example illustrates a method of treating a subject with the COX-2 selective inhibitor compound 2-(4-ethoxyphenyl)-4-methyl-1-(4-sulfamoylphenyl)pyrrole comprising: measuring the level of PGEM in a biological sample collected from the subject prior to administration of the compound, determining that the PGEM level in the sample is higher than about 7 ng/mg for a female subject or higher than about 12 ng/mg for a male subject, and administering to the subject the COX-2 selective inhibitor.

### Example 8

### Treatment with a combination comprising 2-(4-ethoxyphenyl)-4-methyl-1-(4-sulfamoylphenyl)pyrrole and a second agent or therapy based n PGEM level higher than 7 ng/mg for a female subject or higher than about 12 ng/mg for a male subject

This example illustrates a method of treating a subject with a combination comprising the COX-2 selective inhibitor compound 2-(4-ethoxyphenyl)-4-methyl-1-(4-sulfamoylphenyl)pyrrole and a second agent or therapy, the method comprising: measuring the level of PGEM in a biological sample collected from the subject prior to administration of the compound, determining that the PGEM level in the sample is higher than about 7 ng/mg for a female subject or higher than about 12 ng/mg for a male subject, and administering to the subject the COX-2 selective inhibitor.

### Example 9

### Treatment with a combination 2-(4-ethoxyphenyl)-4-methyl-1-(4-sulfamoylphenyl)pyrrole and a second agent or therapy based on PGEM ratio higher than 1.2

This example illustrates a method of treating a subject with a combination comprising the COX-2 selective inhibitor compound 2-(4-ethoxyphenyl)-4-methyl-1-(4-sulfamoylphenyl)pyrrole and a second agent or therapy the method comprising: measuring the level of PGEM in a first biological sample collected from the subject prior to administration of the compound, measuring the level of PGEM in a second biological sample collected from the subject after administration of the second agent or therapy, and determining a ratio by dividing the level of PGEM in the second sample by the level of PGEM in the first sample, determining that the ratio is higher than about 1.2 and administering to the subject the combination of the COX-2 selective inhibitor compound 2-(4-ethoxyphenyl)-4-methyl-1-(4-sulfamoylphenyl)pyrrole and the second agent or therapy.

### Example 10

### Assessing reduction of COX-2 expression for a subject undergoing therapy with the 2-(4-ethoxyphenyl)-4-methyl-1-(4-sulfamoylphenyl)pyrrole

This example illustrates a method of assessing reduction of COX-2 expression in a subject undergoing therapy with the COX-2 selective inhibitor compound 2-(4-ethoxyphenyl)-4-methyl-1-(4-sulfamoylphenyl)pyrrole the method comprising: measuring the PGEM level in the subjects urine at least a first and a second point in time to produce at least a first level of PGEM and a second level of PGEM, wherein the at least first and second points in time are separated from each other by an interval of at least about five days, and wherein a decrease in the second level of PGEM compared to the first level of PGEM is indicative of decreased COX-2 expression.

### Example 11

### Assessing reduction of COX-2 expression for a subject undergoing therapy with a combination comprising 2-(4-ethoxyphenyl)-4-methyl-1-(4-sulfamoylphenyl)pyrrole and a second agent or therapy

This example illustrates a method of assessing reduction of COX-2 expression in a subject undergoing therapy with a combination comprising the COX-2 selective inhibitor compound 2-(4-ethoxyphenyl)-4-methyl-1-(4-sulfamoylphenyl)pyrrole and a second agent or therapy the method comprising: measuring the PGEM level in the subjects urine at least a first and a second point in time to produce at least a first level of PGEM and a second level of PGEM, wherein the at least first and second points in time are separated from each other by an interval of at least about five days, and wherein a decrease in the second level of PGEM compared to the first level of PGEM is indicative of decreased COX-2 expression.

### Example 12

### Assessing response to treatment for a subject undergoing therapy with 2-(4-ethoxyphenyl)-4-methyl-1-(4-sulfamoylphenyl)pyrrole

This example illustrates a method of assessing response to treatment in a subject undergoing therapy with the COX-2 selective inhibitor compound 2-(4-ethoxyphenyl)-4-methyl-1-(4-sulfamoylphenyl)pyrrole the method comprising: measuring the PGEM level in the subjects urine at least a first and a second point in time to produce at least a first level of PGEM and a second level of PGEM, wherein the at least first and second points in time are separated from each other by an interval of at least about five days, and wherein a decrease in the second level of PGEM compared to the first level of PGEM is indicative of positive response to treatment.

### Example 13

### Assessing response to treatment for a subject undergoing therapy with a combination comprising 2-(4-ethoxyphenyl)-4-methyl-1-(4-sulfamoylphenyl)pyrrole and a second agent or therapy

This example illustrates a method of assessing response to treatment in a subject undergoing therapy with a combination comprising the COX-2 selective inhibitor compound 2-(4-ethoxyphenyl)-4-methyl-1-(4-sulfamoylphenyl)pyrrole and a second agent or therapy the method comprising: measuring the PGEM level in the subjects urine at least a first and a second point in time to produce at least a first level of PGEM and a second level of PGEM, wherein the at least first and second points in time are separated from each other by an interval of at least about five days, and wherein a decrease in the second level of PGEM compared to the first level of PGEM is indicative of positive response to treatment.

### Example 14

### Treatment with a combination comprising 2-(4-ethoxyphenyl)-4-methyl-1-(4-sulfamoylphenyl)pyrrole and a second agent or therapy provides an enhanced treatment compared to administering the COX-2 selective inhibitor alone or the second agent or treatment alone

This example illustrates a method of treating a subject with a combination comprising the COX-2 selective inhibitor compound 2-(4-ethoxyphenyl)-4-methyl-1-(4-sulfamoylphenyl)pyrrole and a second agent or therapy, the method comprising: measuring the level of PGEM in a biological sample collected from the subject prior to administration of the compound, determining that the PGEM level in the sample is higher than a predetermined value and administering to the subject the COX-2 selective inhibitor; wherein administering the combination to the patient provides an enhanced treatment compared to administering the COX-2 selective inhibitor alone or the second agent or treatment alone.

### Example 15

### Treatment with a combination comprising 2-(4-ethoxyphenyl)-4-methyl-1-(4-sulfamoylphenyl)pyrrole and a second agent or therapy allows administering the combination at a lower dosage compared to administering the COX-2 selective inhibitor alone or the second agent or treatment alone

This example illustrates a method of treating a subject with a combination comprising the COX-2 selective inhibitor compound 2-(4-ethoxyphenyl)-4-methyl-1-(4-sulfamoylphenyl)pyrrole and a second agent or therapy, the method comprising: measuring the level of PGEM in a biological sample collected from the subject prior to administration of the compound, determining that the PGEM level in the sample is higher than a predetermined value and administering to the subject the COX-2 selective inhibitor wherein the combination is administered at a lower dosage compared to administering the COX-2 selective inhibitor alone or the second agent or treatment alone.

### Example 16

### Treatment with a combination comprising 2-(4-ethoxyphenyl)-4-methyl-1-(4-sulfamoylphenyl)pyrrole and a second agent or therapy with reduced side effects compared to administering the COX-2 selective inhibitor alone or the second agent or treatment

This example illustrates a method of treating a subject with a combination comprising the COX-2 selective inhibitor compound 2-(4-ethoxyphenyl)-4-methyl-1-(4-sulfamoylphenyl)pyrrole and a second agent or therapy, the method comprising: measuring the level of PGEM in a biological sample collected from the subject prior to administration of the compound, determining that the PGEM level in the sample is higher than a predetermined value and administering to the subject the COX-2 selective inhibitor wherein administering the combination to the patient reduces side effects compared to administering the COX-2 selective inhibitor alone or the second agent or treatment alone.

### Example 17

### Treatment with a combination comprising 2-(4-ethoxyphenyl)-4-methyl-1-(4-sulfamoylphenyl)pyrrole and a second agent or therapy with reduced resistance to the second agent compared to administering the second agent or treatment alone

This example illustrates a method of treating a subject with a combination comprising the COX-2 selective inhibitor compound 2-(4-ethoxyphenyl)-4-methyl-1-(4-sulfamoylphenyl)pyrrole and a second agent or therapy, the method comprising: measuring the level of PGEM in a biological sample collected from the subject prior to administration of the compound, determining that the PGEM level in the sample is higher than a predetermined value and administering to the subject the COX-2 selective inhibitor wherein administering the combination to the patient reduces resistance to the second agent compared to administering the second agent or treatment alone.

1. A method of selecting a subject for therapy with the COX-2 selective inhibitor compound 2-(4-ethoxyphenyl)-4-methyl-1-(4-sulfamoylphenyl)pyrrole comprising: measuring the level of PGEM in a biological sample collected from the subject prior to administration of the compound 2-(4-ethoxyphenyl)-4-methyl-1-(4-sulfamoylphenyl)pyrrole, determining that the PGEM level in the sample is higher than a predetermined value and selecting the subject for therapy with the COX-2 selective inhibitor 2-(4-ethoxyphenyl)-4-methyl-1-(4-sulfamoylphenyl)pyrrole.

2.-236. (canceled)

237. A method of selecting a subject for therapy with the COX-2 selective inhibitor compound 2-(4-ethoxyphenyl)-4-methyl-1-(4-sulfamoylphenyl)pyrrole or a combination comprising the COX-2 selective inhibitor compound 2-(4-ethoxyphenyl)-4-methyl-1-(4-sulfamoylphenyl)pyrrole and a second agent or therapy, the method comprising: measuring the level of PGE2 metabolite in a biological sample collected from the subject prior to administration of the compound 2-(4-ethoxyphenyl)-4-methyl-1-(4-sulfamoylphenyl)pyrrole, determining that the PGE2 metabolite level in the sample is higher than a predetermined value and selecting the subject for therapy with the COX-2 selective inhibitor compound 2-(4-ethoxyphenyl)-4-methyl-1-(4-sulfamoylphenyl)pyrrole or the combination comprising the COX-2 selective inhibitor 2-(4-ethoxyphenyl)-4-methyl-1-(4-sulfamoylphenyl)pyrrole and the second agent or therapy.

238. A method of selecting a subject for therapy with a combination comprising the COX-2 selective inhibitor compound 2-(4-ethoxyphenyl)-4-methyl-1-(4-sulfamoylphenyl)pyrrole and a second agent or therapy the method comprising: measuring the level of PGE2 metabolite level in a first biological sample collected from the subject prior to administration of the compound 2-(4-ethoxyphenyl)-4-methyl-1-(4-sulfamoylphenyl)pyrrole, measuring the level of PGE2 metabolite level in a second biological sample collected from the subject after administration of the second agent or therapy, and calculating a ratio by dividing the level of PGE2 metabolite level in the second sample by the level of PGE2 metabolite level in the first sample, determining that the ratio is higher than a predetermined value and selecting the subject for therapy with the combination of the COX-2 selective inhibitor compound 2-(4-ethoxyphenyl)-4-methyl-1-(4-sulfamoylphenyl)pyrrole and the second agent or therapy.

239. A method of treating a subject with the COX-2 selective inhibitor compound 2-(4-ethoxyphenyl)-4-methyl-1-(4-sulfamoylphenyl)pyrrole or a combination comprising the COX-2 selective inhibitor compound 2-(4-ethoxyphenyl)-4-methyl-1-(4-sulfamoylphenyl)pyrrole and a second agent or therapy, the method comprising: measuring the level of PGE2 metabolite in a biological sample collected from the subject prior to administration of the compound 2-(4-ethoxyphenyl)-4-methyl-1-(4-sulfamoylphenyl)pyrrole, determining that the PGE2 metabolite level in the sample is higher than a predetermined value and administering to the subject the combination comprising the COX-2 selective inhibitor 2-(4-ethoxyphenyl)-4-methyl-1-(4-sulfamoylphenyl)pyrrole and the second agent or therapy.

240. A method of treating a subject with a combination comprising the COX-2 selective inhibitor compound 2-(4-ethoxyphenyl)-4-methyl-1-(4-sulfamoylphenyl)pyrrole and a second agent or therapy the method comprising: measuring the level of PGE2 metabolite in a first biological sample collected from the subject prior to administration of the compound, measuring the level of PGE2 metabolite in a second biological sample collected from the subject after administration of the second agent or therapy, and calculating a ratio by dividing the level of PGE2 metabolite in the second sample by the level of PGE2 metabolite in the first sample, determining that the ratio is higher than a predetermined value and administering to the subject the combination of the COX-2 selective inhibitor compound 2-(4-ethoxyphenyl)-4-methyl-1-(4-sulfamoylphenyl)pyrrole and the second agent or therapy.

241. The method of any of claims 237 wherein the PGE2 metabolite is PGEM.

242. The method of any of claims 238 wherein the PGE2 metabolite is PGEM.

243. The method of any of claims 239 wherein the PGE2 metabolite is PGEM.

244. The method of any of claims 240 wherein the PGE2 metabolite is PGEM.
